(19) United States
(12) Patent Application Publication
(10) Pub. No.: US 2023/0137590 A1
(43) Pub. Date: May 4, 2023

(54) COMPOSITIONS AND METHODS FOR PRODUCING DISEASE SUPPRESSION

(71) Applicants: Richard Louis JASONI, Reno, NV (US); David Charles VUONO, Reno, NV (US); John Albert ARNONE, III, Reno, NV (US); Tu Biomics, Inc., Reno, NV (US); Board of Regents of the Nevada System of Higher Education on behalf of the Desert Research Institute, Reno, NV (US)

(72) Inventors: John Albert ARNONE, III, Reno, NV (US); Richard Louis JASONI, Reno, NV (US); David Charles VUONO, Reno, NV (US); Brian SPEICHER, Reno, NV (US)

(73) Assignees: Tu Biomics, Inc., Reno, NV (US); Board of Regents of the Nevada System of Higher Education on behalf of the Desert Research Institute, Reno, NV (US)

(21) Appl. No.: 17/908,514

(22) PCT Filed: Mar. 2, 2021
(86) PCT No.: PCT/US2021/020427

§ 371 Date: Aug. 31, 2022

Related U.S. Application Data
(60) Provisional application No. 62/984,956, filed on Mar. 4, 2020.
(60) Provisional application No. 62/992,364, filed on Mar. 20, 2020.
(60) Provisional application No. 63/110,517, filed on Nov. 6, 2020.

(51) Int. Cl. C12N 1/20; A01P 3/00; A01P 15/00; A01N 37/36; A01N 59/00; A01N 59/06; A01N 59/02; A01N 59/26; A01N 59/10

(52) U.S. Cl. CPC C12N 1/20; A01N 37/36; A01N 59/00; A01N 59/02; A01N 59/06; A01N 59/10; A01N 59/26; A01P 3/00; A01P 15/00; C12N 2500/02; C12N 2500/34

(57) ABSTRACT

Provided herein are compositions, systems, and methods for treating plants, soil, fungal, and/or pathogens. More specifically, the present disclosure relates to compositions having one or more microbial metabolites from a microbial cell bath mixture for the treatment of harmful plant, agriculture, and/or soil pathogens, and also relates to methods of making and using the compositions.

### CROSS-REFERENCE TO RELATED APPLICATIONS

This application claims the benefit of the following U.S. Provisional Applications No.: 62/984,956, filed Mar. 4, 2020; 62/992,364, filed Mar. 20, 2020; and 63/110,517, filed Nov. 6, 2020; the entire contents of which are incorporated herein by reference.

### BACKGROUND

At the scale of industrial agriculture, where disease pressure has an economically meaningful impact globally, a continually narrowing selection of agrochemicals continues to be applied with increasing examples of pathogen resistance, and with deleterious effects on soil health, the environment, and human health. Efforts have been made to develop more sustainable ways of controlling agricultural diseases, however, the methods are difficult to implement or yield unpredictable results. For example, anaerobic soil disinfestation (ASD) is characterized by inconsistent results and is prohibitive in scale and expense, thereby limiting opportunities for optimization or application in commercial scale agriculture.

Therefore, there remains a need for the development of safe and environmentally friendly compositions and methods for effectively and economically promoting plant health and controlling and/or mitigating the growth of pathogens that have a deleterious effect in global food production and plant health.

### SUMMARY

As described below, the present invention features methods and compositions for inhibiting the growth of a plant (e.g., a crop plant, tree, ornamental plant, lettuce, Allium plant, or turf) fungal pathogen (e.g., Botrytis cinerea, Colletotrichum aculaturn, Fusarium oxysporum sp. fragariae, Macrophomina phaseolina, Phytophihora cactorum, Pythium uncinulaium, Rhizoeionia solani, Sclerotinia sclerotiorum, Sclerotium cepivorum, Sclerotinia minor, or Verticillium dahliae), and methods for preparation of the compositions.

In one aspect, the invention features a method for preparing a biocontrol agent. The method involves a) aerobically incubating a mixture containing a soil microbiome and a solution for at least about 1-3 days; b) anaerobically incubating the mixture for at least about 1-3 days; and c) removing solids from the mixture and retaining a conditioned media containing soil microbiome metabolites, thereby preparing a biocontrol agent. In embodiments, the soil microbiome contains bacteria selected from one or more of Acidobacteria, Actinobacteria, Bacteroidetes, Chlamydiae, Chloroflexi, Cyanobacteria, Deinococcus-Thermus, Firmicutes, Gemmatimonadetes, Hydrogenedentes, Nitrospirae, Parcubacteria, Planctomycetes, Proteobacteria, Saccharibacteria, Spirochaetes, Tenericutes, Thaumarchaeota, and Verrucomicrobia.

In one aspect, the invention features a method for preparing a biocontrol agent. The method involves a) aerobically incubating a mixture containing a soil microbiome in solution, the soil microbiome containing two or more bacteria selected from one or more of Acidobacteria, Actinobacteria, Bacteroidetes, Chlamydiae, Chloroflexi, Cyanobacteria, Deinococcus-Thermus, Firmicutes, Gemmatimonadetes, Hydrogenedentes, Nitrospirae, Parcubacteria, Planctomycetes, Proteobacteria, Saccharibacteria, Spirochaetes, Tenericutes, Thaumarchaeota, and Verrucomicrobia; b) anaerobically incubating the soil microbiome in solution for at least about 1-3 days; and c) removing microbial cells from the mixture and retaining a conditioned media containing soil microbiome metabolites, thereby preparing a biocontrol agent.

In any of the above aspects the soil microbiome is present in a solid matrix and the ratio of solid matrix to solution is at least about 1:10. In embodiments, the ratio of solid matrix to solution is at least about 1:20. In embodiments, the solid matrix contains soil, compost, and/or another medium that supports the viability and/or growth of the soil microbiome. In embodiments, the compost contains humic compost, earthworm castings, manure, or other organic materials. In embodiments, the mixture contains at least about 5% to 20% by volume of the solid matrix. In embodiments, the solid matrix is incubated in a bioreactor containing a vessel having a perforated surface, the vessel contains the solid matrix.

In any of the above aspects, the solution contains water and one or more ingredients selected from one or more of carbohydrate, salt, a buffering agent, minerals, and vitamins. In embodiments, the carbohydrate is sugar. In embodiments, the sugar is added to the solution at the start of incubation, 1-3 days after the start of incubation, or periodically during the course of incubation. In embodiments, the sugar contains glucose and/or fructose. In embodiments, the sugar contains glucose and fructose at a weight ratio of about 1:1.

In any of the above aspects, the aerobic incubation is carried out for at least about 1 day. In any of the above aspects, the aerobic incubation is carried out for at least about 2 or 3 days. In any of the above aspects, the aerobic incubation is carried out for at least about 3-5 days, but no longer than 14 days.

In any of the above aspects, a gas containing oxygen is introduced to the solution during the aerobic incubation. In embodiments, the gas is introduced at a flow rate of at least about 4 ft³/min.

In any of the above aspects, the anaerobic incubation is carried out for at least about 1 day. In any of the above aspects, the anaerobic incubation is carried out for about 7-10 days.

In any of the above embodiments, the aerobic and/or anaerobic incubation is carried out at a temperature between about 16° C. to about 35° C. In any of the above embodiments, the aerobic and/or anaerobic incubation is carried out at a temperature selected from one or more of about 18° C., about 19° C., about 20° C., about 21° C., and about 22° C.

In any of the above aspects, oxygen levels during aerobic incubation are greater than 0.2 mg/L. In any of the above aspects, oxygen levels during anaerobic incubation are less than about 0.2 mg/L.

In any of the above aspects, the pH of the mixture is neutral at the start of aerobic and/or anaerobic incubation.

In any of the above aspects, solids present in the mixture are removed by centrifugation or filtering. In embodiments, filtering is carried out using a filter containing a nominal pore size of less than about 0.25 µm. In embodiments, the nominal pore size is less than about 0.05 µm.

In any of the above aspects, at least about 50% of bacteria present after the aerobic incubation and/or the anaerobic incubation, as measured by 16S rRNA gene sequencing, are Firmicutes and/or Gammaproteobacteria. In any of the above aspects, the cell bath mixture containing a prokaryotic species relative abundance, as measured by 16S rRNA gene sequencing, of Bacilli, Clostridia, and/or Gammaproteobacteria of at least about 20% after the aerobic incubation and/or the anaerobic incubation. In any of the above aspects, the top 5 prokaryotic taxa represented in the cell bath mixture by relative abundance, as measured by 16S rRNA gene sequencing, comprises Bacillus, Clostridium, and Leuconostoc during or at the termination of the resting phase.

In any of the above aspects, the biocontrol agent contains lactate, acetate, and propionate. In any of the above aspects, the method further involves concentrating the biocontrol agent.

In one aspect, the invention features a biocontrol agent prepared by the method of any of the above aspects,

In one aspect, the invention features a liquid biocontrol agent containing metabolites of a soil microbiome, where the soil microbiome contains two or more bacteria selected from one or more of Acidobacteria, Actinobacteria, Bacteroidetes, Chlamydiae, Chloroflexi, Cyanobacteria, Deinococcus-Thermus, Firmicutes, Gemmatimonadetes, Hydrogenedentes, Nitrospirae, Parcubacteria, Planctomycetes, Proteobacteria, Saccharibacteria, Spirochaetes, Tenericutes, Thaumarchaeota, and Verrucomicrobia. The liquid biocontrol agent has anti-fungal activity.

In one aspect, the invention features a kit for use in the method of any of the above aspects. The kit contains the biocontrol agent of any one of the above aspects. In embodiments, the kit further contains a spray bottle, a sprayer, a nozzle, or a drip line for applying the biocontrol agent.

In one aspect, the invention features a method of controlling a fungal pathogen. The method involves contacting the fungal pathogen with a biocontrol agent of any of the above aspects, thereby controlling the fungal pathogen.

In one aspect, the invention features a method of controlling a fungal pathogen. The method involves contacting a soil or plant containing the fungal pathogen with a biocontrol agent containing metabolites of a soil microbiome, where the soil microbiome contains two or more bacteria selected from one or more of Acidobacteria, Actinobacteria, Bacteroidetes, Chlamydiae, Chloroflexi, Cyanobacteria, Deinococcus-Thermus, Firmicutes, Gemmatimonadetes, Hydrogenedentes, Nitrospirae, Parcubacteria, Planctomycetes, Proteobacteria, Saccharibacteria, Spirochaetes, Tenericutes, Thaumarchaeota, and Verrucomicrobia.

In any of the above aspects, the soil microbiome contains a prokaryotic species relative abundance, as measured by 16S rRNA gene sequencing, of Proteobacteria, Firmicutes, and Actinobacteria of at least 30%.

In any of the above aspects, the plant belongs to the Allium genus. In any of the above aspects, the plant is selected from one or more of Allium sativum, Allium cepa, Allium chinense, Allium stipitatum, Allium schoenoprasiim, Allium tuberosum, Allium fistulosum, or Allium ampeloprasum. In any of the above aspects, the plant is selected from one or more of peas, lettuce, broccoli, beans, grape, strawberry, and raspberry.

In any of the above aspects, the fungal pathogen belongs to a genus selected from one or more of Botrytis, Colletotrichum, Fusarium, Macrophomina, Phytophthora, Pythium, Rhizoctonia, Sclerotinia, Sclerotiniaceae. Sclerotium, and Verticillium. In any of the above aspects, the fungal pathogen is selected from one or more of Botrytis cinerea, Colletotrichum acutatum, Fusarium oxysporum sp. fragariae, Macrophomina phaseolina, Phytophthora caclorum, Pythium uncinulatum, Rhizoctonia solani, Sclerotinia sclemtiorum, Sclerotium cepivorum, Sclerotinia minor, and Verticillium dahliae. In any of the above aspects, the fungal pathogen is Sclerotinia minor or Sclerotinia sclerotiorum. In any of the above aspects, the fungal pathogen is Sclerotium cepivorum.

In any of the above aspects, the contacting involves base spray or drip application. In any of the above aspects, contacting occurs at least 3 times. In any of the above aspects, each contacting occurs at least about 4 days from a previous contacting. In any of the above aspects, the biocontrol agent is applied to the soil in an amount of at least about 1000 gal/acre per application. In any of the above aspects, contacting is associated with increased agricultural yield relative to the agricultural yield of untreated soil.

The invention provides methods and compositions for inhibiting the growth of fungal pathogens, and methods for preparation of the compositions. Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviations, per the practice in the art. Alternatively, “about” can mean a range of up to 20%, up to 10%, up to 5%, and up to 1% of a given value. Alternatively, particularly with respect to biological systems or processes, the term can mean within an order of magnitude, within 5-fold, and within 2-fold, of a value. Where particular values are described in the application and claims, unless otherwise stated the term “about” meaning within an acceptable error range for the particular value should be assumed.

By “aerobic incubation” is meant an incubation in which oxygen is actively introduced to a mixture being incubated. In embodiments, aerobic incubation involves bubbling a gas containing oxygen into a mixture. Active introduction typically involves bubbling or agitation of a mixture to increase the concentration of oxygen in the mixture

By “anaerobic incubation” is meant an incubation in which no oxygen is actively introduced to a mixture being incubated.

By “biocontrol agent” is meant a composition produced by the methods described herein for control of growth of a fungal pathogen.

By “compost” is meant a mixture of decayed or decaying organic matter. In embodiments compost can comprise dead leaves or manure. In embodiments the compost is earthworm compost, where earthworm compost is a composition resulting from the decomposition of organic matter by worms. In embodiments, earthworm compost contains or is worm castings.

By “acetate” or “acetic acid” is meant a compound having the formula C₂H₄O₂, corresponding to CAS Number 64-19-7, and having the structure

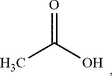

and agronomically acceptable salts thereof. The salt can be a lithium, sodium, or potassium salt.

By “agent” is meant any small molecule chemical compound. The small molecule chemical compound can be an organic acid (e.g., lactic acid and/or acetic acid).

By “agricultural field” is meant an area of land under cultivation or to be used for cultivating crops.

By “ameliorate” is meant decrease, suppress, attenuate, diminish, arrest, or stabilize the development or progression of a disease. In some embodiments, the disease is associated with a fungal pathogen (e.g., Sclerotium cepivorum, Botrytis cinerea). In some embodiments, the disease is gray mold or white rot.

By “bioreactor” is meant a container suitable for incubating a mixture comprising microbes. In embodiments, the bioreactor is a tank (e.g., an open-top water storage tank). In embodiments, the mixture contains a solution and a soil microbiome.

By “carrier” is meant a substance that functions to facilitate the application of a composition to a plant or soil.

In this disclosure, “comprises,” “comprising,” “containing” and “having” and the like can have the meaning ascribed to them in U.S. Patent law and can mean ” includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “concentrate” is meant a composition containing a high concentration of components because of lack of a solvent. A concentrate can be referred to as 2X, 3X, 4X, 5X, etc. depending on how many-fold the concentrate must be diluted using a solvent (e.g., water) to obtain a target, or working, concentration of the composition components. The concentrate can be a 1.5X, 2X, 3X, 4X, 5X, 10X, 15X, 20X, 25X, 50X, 75X, 100X, 150X, 200X, 250X, 300X, 500X, 750X, or 1,000X concentrate.

As used herein, “conditioned media” refers to a solution harvested from a mixture in which a microbial community was incubated. In embodiments, harvesting involves removing solids from the mixture, optionally by filtration or by centrifugation. In embodiments, harvesting involves removing microbes from the mixture.

By “consist essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight.

“Detect” refers to identifying the presence, absence or amount of the analyte to be detected.

By “disease” is meant any condition or disorder that damages or interferes with soil or plant function. The normal function of a soil includes the ability to sustain growth of a disease-free plant therein. The disease can be caused by a plant pathogen (e.g., fungi). In some embodiments, the plant disease is white rot or gray mold Pathogenic fungi include, for example, Sclerotium cepivorum and Botrytis cinerea. By “effective amount” is meant the amount of an agent required to ameliorate the symptoms of a disease relative to an untreated soil or plant. The effective amount of active compound(s) used to practice the present invention for treatment or prevention of a fungal disease (e.g., white rot, gray mold) varies depending upon the manner of administration and the plant and/or soil being treated. Such amount is referred to as an “effective” amount. In some embodiments, an effective amount is the amount required to inhibit fungal growth or to kill the fungus.

By “growth medium” is meant a solid, liquid, or semi-solid that functions to support growth of a plant. In some embodiments, the growth medium is a soil. In some embodiments, the growth medium contains soil, bark, clay (e.g., calcined clays), coir pith, green compost, peat (e.g., black peat or white peat), perlite, rice hulls, sand, grit, wood fibers, peat, vermiculite, leaf mold, sawdust, bagasse, expanded polystyrene, urea formaldehydes, or a combination thereof. In some embodiments, the growth medium is a hydroponic growth medium.

By “L-lactate” or “L-lactic acid” is meant a compound having the chemical formula C₃H₆O₃, corresponding to CAS Number 79-33-4, having the structure

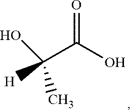

and agronomically acceptable salts thereof. The salt can be a lithium, sodium, or potassium salt.

By “D-lactate” or “D-lactic acid” is meant a compound having the chemical formula C₃H₆O₃, corresponding to CAS Number 10326-41-7, and having the structure

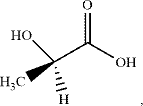

and agronomically acceptable salts thereof. The salt can be a lithium, sodium, or potassium salt.

The term “lacate” can refer to D-lactate, L-lactate, or mixtures thereof.

By “mitigate” is meant alleviating or reducing a pathogen or harmful effects thereof. As used herein “eliminate” refers to eradication of a pathogen or eradication of harmful effects of the pathogen. As used herein “inhibit” refers to a reduction in an amount of a pathogen or a reduction in harmful effects of the pathogen. As used herein “kill” refers to the destruction of a pathogen or the permanent and irreversible elimination of the capacity thereof to proliferate or reproduce. As used herein “slow” refers to reducing the spread of a pathogen or reducing the rate at which harmful effects of the pathogen are established or increase. The terms mitigate, eliminate, inhibit, kill, slow, control, or prevent can include partial or complete mitigation, elimination, inhibition, death, slowing, control, or prevention of the pathogen or of harmful effects of the pathogen. For example, the mitigation, elimination, inhibition, death, slowing, control, or prevention can be of 10%, 20%, 30%, 40%, 50%, 60%, 70%, 80%, 90%, 100%, or an amount within a range defined by any two of the aforementioned values.

By “neutral pH” is meant a pH of from about 6 to about 8. In embodiments a neutral pH is a pH from about 6.5 to about 7.5 or of about 7.

By “nominal pore size” is meant the ability of a filter to retain the majority of particles at the rated pore size and larger. In embodiments, about or at least about 50%, 60%, 70%, 80%, 90%, or 100% of particles larger than the nominal pore size are retained.

As used herein, “obtaining” as in “obtaining an agent” includes synthesizing, purchasing, or otherwise acquiring the agent.

By “parts per million (ppm)” is meant a unit of concentration equivalent to mg/L or g/m³, where density of a liquid is estimated at about 1 g/ml, or to mg/kg. For example, 1 L of an aqueous solution containing 100 mg lactate may be described as containing 100 ppm lactate. As a further example, a 1 kg soil sample containing 100 mg lactate may be described as containing 100 ppm lactate.

By “pathogen” is meant an organism that causes a disease in a plant. In some embodiments, the pathogen is a fungal pathogen (e.g., Sclerotium cepivorum, Botrytis cinerea). In some embodiments, the disease is white rot or gray mold. In some embodiments, the fungal pathogen is adversely affecting the growth of plants, the appearance of plants, the production and yield of plant-based food, the appearance of plant-based food, the preservation of plant-based food, the cultivation of plants. In some embodiments, the pathogen is any and all forms of anthracnose or any and all types of Botrytis, Fusarium (including F. oxysporum f. sp. Fragariae, Cubense or F. solani), Thielavopsis (root rot), Mycosphaerella (including M. fijiensis and M. musicola), Verticillium (including V. dahlia), Macrophomina phaseolina, Magnaporthe grisea, Sclerolhtia sderotiorum, Sclerotium cepivorum (alternatively, Stromatina cepivora), Ustilago, Rhizoctonia (including R. solani), Cladosporium, Collelotrichum (including C. coccodes, C. acutatum, C. truncatum, or C. gloeosporoides), Trichoderma (including T. viride or T. harzianum), Helminthosporium (including H. solani), Alternaria (including A. solani or A. alternata), Aspergillus (including A. niger or A. fumigatus), Phakospora pachyrhizi, Puccinia, Pythium, oomycetes (including Phytophthora), and Armillaria. In some embodiments, the plant pathogen belongs to the family class Leotiomycetes, to the order Helotiales, and/or to the family Sclerotiniaceae.

The term “plant” includes all organisms of the plant kingdom, as well as their cells, tissues, and products. Accordingly, the term plant includes seeds, leaves, stems, roots, fruit, and the like.

As used herein, the terms “prevent,” “preventing,” “prevention,” “prophylactic treatment” and the like refer to reducing the probability of developing a disease (e.g., white rot, gray mold) in a plant or soil, that does not have, but is at risk of or susceptible to developing the disease.

By “propionate” or “propionic acid” is meant a compound having the formula C₃H₆O₂, corresponding to CAS Number 79-09-04 or 72-03-7, and having the structure

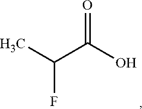

and agronomically acceptable salts thereof. The salt can be a lithium, sodium, or potassium salt.

By “reduces” is meant a negative alteration of at least 5%, 10%, 25%, 50%, 75%, or 100%.

By “reference” is meant a standard or control condition. In one embodiment, a reference is a plant, soil, or other medium that comprises a fungal pathogen (e.g., Sclerotium cepivorum, Botrytis cinereal), but that is not contacted with a composition of the invention (e.g., a biocontrol agent).

By “sterile composition” is meant a composition free from the presence of viable organisms.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, “soil” refers to a composition that functions to provide structural support to plants and functions as a source of water and nutrients for the plants. A soil can contain a mixture of inorganic (e.g., sand, silt, clay, gravel) and organic materials. The soil can contain particles greater than 2 mm in diameter (gravel), particles from about 0.2 mm in diameter to about 2 mm in diameter (coarse sand), particles from about 0.02 mm in diameter to about 0.2 mm in diameter (fine sand), particles from about 0.002 mm in diameter to about 0.02 mm in diameter (silt), particles of less than 0.002 mm in diameter (clay) or various combinations thereof.

As used herein, “soil microbiome” refers to a collection of microbial species containing a set or subset of microbial species represented in a soil or compost sample.

In some embodiments, as non-limiting examples, a soil microbiome may contain bacteria selected from one or more of the following: Acidobacteria, Actinobacteria, Bacteroidetes, Chlamydiae, Chloroflexi, Cyanobacteria, Deinococcus-Thermus, Firmicutes, Gemmatimonadetes, Hydrogenedentes, Nitrospirae, Parcubacteria, Planctomycetes, Proteobacteria, Saccharibacteria, Spirochaetes, Tenericutes, Thaumarchaeota, and Verrucomicrobia.

As used herein, the terms “treat,” treating,” “treatment,” and the like refer to reducing or ameliorating a disease from a soil or plant.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 is a flow-chart illustrating one embodiment of a process for producing a biocontrol agent.

FIG. 2 is a stacked bar graph illustrating the taxonomic composition of an inoculum used for preparing a biocontrol agent In the figure, the taxa are listed in the legend in the same order in which they occur in the bar graph.

FIG. 3 illustrates a process time course of conditions measured during preparation of the first batch of the biocontrol agent. In the figures, “Aerobic” corresponds to the aeration phase and “Anaerobic” corresponds to the resting phase.

FIG. 4 illustrates a process time course of solution conditions measured during preparation of the second batch of the biocontrol agent.

FIG. 5 illustrates taxa prevalence plots by average count abundance.

FIG. 6 illustrates a taxa comparison plot. In the figure, the taxa are listed in the legend in the same order in which they occur in the bar graph. Not all bars contain all taxa listed in the legend.

FIG. 7 illustrates an increase abundance of Clostridia after termination of the aeration phase at day 3. In the figure, the taxa are listed in the legend in the same order in which they occur in the bar graph. Not all bars contain all taxa listed in the legend.

FIGS. 8A-8B illustrate alpha diversity by day and redox during preparation of a biocontrol agent. Throughout the figures the sample designated as having an oxic redox corresponds to a sample taken at the termination of the aeration phase (day 3) and the sample designated as having an anoxic redox corresponds to a sample taken at the termination of the resting phase (day 10). In FIG. 8B, the upper dots represent batch 2 and the lower dots represent batch 1.

FIG. 9 presents box-and-whisker plots illustrating the difference in abundance of the top 10 taxa between Oxic and Anoxic growth conditions. For each genera, the box to the left is for anoxic growth conditions and the box to the right is for oxic growth conditions.

FIG. 10 illustrates the community similarity changes from left to right on first PC axis indicating that the community changes with time. In general, in the figure, the data points represent progressively later days from left to right.

FIG. 11 illustrates that the Clostridia group increases during anoxic periods and Gammas and Firmicutes were abundant throughout preparation of a biocontrol agent. In the figure, the only plots containing data points corresponding to taxa other than that indicated in the title of the plot are the “Bacteroidetes” plot (containing Sphingobacteriia and Flavobacteria data points only) and the “Firmicutes” plot (containing Bacilli and Clostridia data points only).

FIG. 12 illustrates statistical testing between oxic and anoxic conditions. In the figure, the circle to the left encircles “oxic” data points and the circle to the right encircles “anoxic” data points. The data point that is not encircled is an “anoxic” data point, and the data point that is intersected by the oxic conditions circle is an “anoxic” data point.

FIG. 13 illustrates canonical correspondence analysis.

FIG. 14 illustrates the top 60 taxa phylogenetic analysis.

FIGS. 15A-15E are a plot and images of petri plates inoculated with Sclerotinia sclerotiorum demonstrating that the biocontrol agent suppresses growth of the fungal pathogen. The images presented in FIGS. 15A-15D were taken at 2, 3, 4, and 7 days post-inoculation, respectively. In each of FIGS. 15A-15D, the upper panel is an image of a negative control petri plate containing water in place of the biocontrol agent and the lower panel is an image of a petri plate containing the biocontrol agent (BCA). For scale, a centimeter ruler is shown in each image. FIG. 15E provides a plot of fungal colony area over time. Error bars represent one standard deviation from the mean.

FIGS. 16A-16E are a plot and images of petri plates inoculated with Sclerotinia minor demonstrating that the biocontrol agent suppresses growth of the fungal pathogen. The images presented in FIGS. 16A-16D were taken at 2, 3, 4, and 7 days post-inoculation, respectively. In each of FIGS. 16A-16D, the upper panel is an image of a negative control petri plate containing water in place of the biocontrol agent and the lower panel is an image of a petri plate containing the biocontrol agent (BCA). For scale, a centimeter ruler is shown in each image. FIG. 16E provides a plot of fungal colony area over time. Error bars represent one standard deviation from the mean

FIGS. 17A-17D are a plot and images of petri plates inoculated with Pythium uncinulatum demonstrating that the biocontrol agent suppresses growth of the fungal pathogen. The images presented in FIGS. 17A-17C were taken at 3, 4, and 7 days post-inoculation, respectively. In each of FIGS. 17A-17C, the upper panel is an image of a negative control petri plate containing water in place of the biocontrol agent and the lower panel is an image of a petri plate containing the biocontrol agent (BCA). For scale, a centimeter ruler is shown in each image. FIG. 17D provides a plot of fungal colony area over time. Error bars represent one standard deviation from the mean.

FIGS. 18A-18E are plots demonstrating that the biocontrol agent was capable of inhibiting growth of Colletotrichum acutatum (FIG. 18A), Fusarium oxysporum (FIG. 18B), Macrophomina phaseolina (FIG. 18C), Phytophthora cactorum (FIG. 18D), and Verticillium dahliae (FIG. 18E) on potato dextrose agar. Growth was measured at each percentage of biocontrol agent evaluated in quintuplicate (N=5). Each line represents growth on potato dextrose agar containing the indicated volumetric percentage of the biocontrol agent. Error bars represent one standard deviation from the mean (N=5)

FIG. 19 is a photograph of two beds within an EcoCELL pot. The left most bed acted as the control, receiving water application while the right bed received the biocontrol agent.

FIG. 20 is a photograph showing black drip tape lines running the length of beds and used to water garlic. The white plastic squares in the three corners of the photograph are the tops of CS616 TDR sensors used to measure volumetric soil water content in the top 20 cm of soil

FIG. 21 is a photograph showing the Mar. 20, 2019 application of white rot infected soil slurry to the EcoCELL pots containing unhealthy soil The infected soil was taken from an quarantined field in San Juan Bautista, California.

FIG. 22 presents photographs of cured garlic bulbs harvested from each seedline within each of three EcoCELL pots. Photos on the right side of each pair of photos show bulbs produced under biocontrol agent treatment, while photos on the left side depict bulbs produced under the negative control water application. The top pair of photos shows bulb yield when garlic was grown in healthy Yerington, Nevada field soil (i.e., no white rot present). The bottom two pairs of photos show bulb yield when garlic was grown in “unhealthy” quarantined Yerington, Nevada field soil (i.e, white rot infected) that was additionally inoculated with infected soil taken from an “unhealthy” quarantined field in San Juan Bautista, California. BCA indicates “biocontrol agent”.

FIG. 23 presents bar graphs presenting harvest data showing the mean (top panel) total number of bulbs produced per seed line for plants growing in healthy (i.e, no white rot present) and diseased (i.e., white rot infected) Yerington, Nevada field soil, and (bottom panel) the mean cured biomass per bulb. Darkly shaded bars represent bulbs grown with the biocontrol agent treatment, while lightly shaded bars depict bulbs produced under the negative control water application. The top pair of photos shows bulb yield when garlic was grown in healthy Yerington, Nevada field soil (i.e., no white rot present). Infected soils were additionally inoculated with infected soil taken from an “unhealthy” quarantined field in San Juan Bautista, California.

FIG. 24 provides images of garlic plants grown in a field.

FIG. 25 is a plot of maximum and minimum air temperatures.

FIG. 26 is a plot of soil temperatures.

FIG. 27 is an annotated image showing the soil disease burden in experimental plots in a field. The disease burden was quantified by sclerotia counts. Plot 2 is the location of the field trial.

FIGS. 28A-28H are bar graphs showing growth of the indicated species on potato dextrose agar with and without addition of the biocontrol agent. Growth was evaluated four and seven days following inoculation (N=3). 0.2 sq. cm was the area of the agar plug used to inoculate the petri plates and, therefore, an area of 0.2 sq. cm corresponds to zero growth. 56.7 sq. cm was the area of the petri dish. Error bars are equal to one standard deviation.

FIG. 29 provides a collection of plots showing efficacy of biocontrol agent (BCA) (solid black circles) against the in vitro growth of Sclerotium, relative to controls (agar containing no BCA, white-filled circles), during the 10 days of preparation of the biocontrol agent batches. Lines in each plot in the top row of plots show the cumulative Sclerotium colony growth areas (cm²) from the time of inoculation through 7 days of growth in petri dishes (mean±SE, n=4 plates per plotted point). Lines in each plot in the bottom row of plots show the relative cumulative Sclerotium colony growth (expressed as a percentage of the control mean) from the time of inoculation through 7 days of growth in petri dishes. Complete efficacy was observed as early as day 4 (after 1 day in the resting or anoxic phase).

### DETAILED DESCRIPTION

The invention features methods and compositions that are useful for inhibiting the growth of fungal pathogens (e.g., Botrytis cinerea, Colletotrichum acutatum, Fusarium oxysporum sp. fragariae, Macrophomina phaseolina, Phytophthora cactorum, Pythium uncinulatum, Rhizoctonia solani, Sclerotinia sclerotiorum, Sclerotium cepivorum, Sclerolillia minor, or Vertieillium dahliae) and methods for preparation of the compositions

The invention is based, at least in part, upon the discovery that disease-suppressive properties of pathogen-free soils are transferrable, and upon the discovery of methods for preparing compositions for transferring these disease-suppressive properties from one soil to another.

In embodiments of the invention, a method to transfer disease-suppressive properties of a soil to another soil with disease conducive properties involves inducing, isolating and/or extracting the biochemical elements (e.g., metabolites produced by a microbial community) that together are associated with disease-suppressive soil. These biochemical elements can be produced by an assortment of aerobic and/or anaerobic microbial taxa associated with disease-suppression. In embodiments, these biochemical elements, as opposed to microbes themselves, are associated with disease suppression when transferred to a soil. In embodiments, the biochemical elements include, as non-limiting examples, organic acids, volatile fatty acids (VFAs), volatile organic compounds (VOCs), secondary metabolites such as non-ribosomal peptide synthases, plant defense activators such as beta-aminobutyric acid (BABA), bacterial lipopolysaccharide, lipoproteins, peptidoglycans, fungal chitins, and the like, and various combinations thereof. Compositions produced by the methods described herein and containing the biochemical elements facilitate transfer of advantageous characteristics (i.e., disease suppression) of disease-suppressive soils to disease-conducive soils to control (e.g., reduce, abate, or eliminate) pathogen growth in the otherwise disease-conductive soils.

Provided herein are compositions and methods for promoting plant health and controlling the growth of pathogens (e.g, Botrytis cinerea, Colletotrichum aculatum, Fusarium oxysporum sp. fragariae, Macrophomina phaseolina, Phytophthora cactorum, Pythium uncinulatum, Rhizoctonia solani, Sclerotinia sclerotiorum, Sclerotium cepivorum, Sclerotinia minor, or Verticillium dahliae) that may have a deleterious effect on plant health. Some embodiments relate to compositions that include one or more microbial metabolites from a microbial cell bath mixture. Some embodiments relate to methods of making the compositions Some embodiments relate to methods of controlling a pathogen or methods of mitigating the deleterious effects of a pathogen.

### Process for Production of a Biocontrol Agent

Aspects provided herein relate to systems and methods of making compositions as described herein.

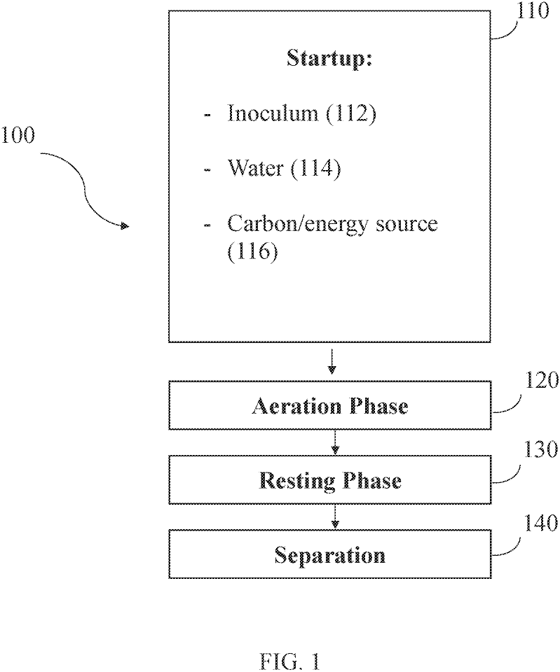

FIG. 1. provides a flow-chart illustrating one embodiment of a process 100 of producing compositions (e.g., a biocontrol agent) as described herein. Features of the process represented in FIG. 1 are referenced herein using the element numbers indicated in FIG. 1 Compositions produced by the methods described herein may be referred to as “biocontrol agents”. In some embodiments, the process 100 comprises a startup 110, an aeration phase 120, a resting phase 130, and a separation 140. In embodiments, the separation 140 involves filtration or centrifugation. In some embodiments startup 110 involves preparing an inoculum 112. In some embodiments, the startup 110 involves obtaining an inoculum 112 and adding water 114 to the inoculum 112 to produce a cell bath mixture. In embodiments, the startup further comprises adding a carbon/energy source 116 (e.g., a sugar) to the cell bath mixture. In some embodiments, the inoculum 112 contains one or more species from the phyla Acidobacteria, Actinobacteria, Bacteroidetes, Chlamydiae, Chloroflexi, Cyanobacteria, Deinococcus-Thermus, Firmicutes, Gemmatimonadetes, Hydrogenedentes, Nitrospirae, Parcubacteria, Planctomycetes, Proteobacteria, Saccharibacteria, Spirochaetes, Tenericutes, Thaumarchaeota, Verrucomicrobia. In some embodiments, the inoculum forms part of a soil-compost mixture. In some embodiments, the soil-compost mixture contains a topsoil, a humic compost, and/or an earthworm compost. In some embodiments, the cell bath mixture further comprises kelp, a fish suspension, feather meal, rock powder, mycorrhizal fungi, amino acids, and/or trace minerals. In some embodiments, the carbon/energy source 116 contains sucrose, dextrose, fructose, a syrup (eg., molasses), an alcohol, an artificial sugar, a derivative thereof, or various combinations thereof. In some embodiments the method comprises measuring conditions (e.g., temperature, pH, electrical conductivity, microbial composition, and oxygen levels) in the cell bath mixture.

In some embodiments, the startup 110 is followed by an aeration phase 120. In some embodiments, the aeration phase 120 comprises aerating the cell bath mixture using a gas composition, optionally air, oxygen, and/or a nitrogen-oxygen gas mixture. In some embodiments, aeration of the cell bath mixture is associated with an increase in oxygen levels in the cell bath mixture and, optionally, the establishment of aerobic conditions in the cell bath mixture. In embodiments, aerobic conditions are established and maintained in the cell bath mixture for about or for at least about 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, or 5 days during the aeration phase. In embodiments, aerobic conditions correspond to oxygen concentrations in the cell bath mixture of about or of at least about 5 ppm, 10 ppm, 15 ppm, 20 ppm, 25 ppm, 30 ppm, 35 ppm, or 40 ppm. In some embodiments, during the aeration phase, the oxygen saturation in the cell bath mixture is about or at least about 0%, 5%, 10%, 15%, 20%, 25%, 30%, 35%, 40%, 45%, 50%, 55%, 60%, 65%, 70%, 75%, 80%, 85%, 90%, 95%, 100%. The aeration phase 120 can have a time duration of about or of at least about 1 hour, 3 hours, 6 hours, 12 hours, 1 day, 2 days, 3 days, 4 days, 5 days, 6 days, 7 days, 8 days, 9 days, 10 days, or a month. In embodiments, the aeration phase 120 has a time duration of no more than about 1 hour, 3 hours, 6 hours, 12 hours, 1 day, 2 days, 3 days, 4 days, 5 days, 6 days, 7 days, 8 days, 9 days, 10 days, or a month.

In some embodiments, the aeration phase 120 is followed by a resting phase 130. In some embodiments, the resting phase 130 is characterized by a lack of aeration and/or low oxygen levels in the cell bath mixture. In embodiments, anaerobic conditions are established in the cell bath mixture during the resting phase 130. In embodiments, anaerobic conditions in the cell bath mixture correspond to an oxygen concentration of less than about 10 ppm, 9 ppm, 8 ppm, 7 ppm, 6 ppm, 5 ppm, 4 ppm, 3 ppm, 2 ppm, 1 ppm, 0.5 ppm, 0.25 ppm, or 0.1 ppm. In some embodiments, during the resting phase, the oxygen saturation in the cell bath mixture is no more than about 0%, 0.1%, 0.2%, 0.3%, 0.4%, 0.5%, 0.6%, 0.7%, 0.8%, 0.9%, 1%, 2%, 3%, 4%, or 5%.

In embodiments, anaerobic conditions are established and maintained in the cell bath mixture for about or for at least about 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, 8 days, 9 days, 10 days, 11 days, 12 days, 13 days, 14 days, or a month during the resting phase. The resting phase 130 can have a time duration of about or of at least about 1 hour, 3 hours, 6 hours, 12 hours, 1 day, 2 days, 3 days, 4 days, 5 days, 6 days, 7 days, 8 days, 9 days, 10 days, 11 days, 12 days, 13 days, 14 days, or a month. In embodiments, the aeration phase 120 has a time duration of no more than about 1 hour, 3 hours, 6 hours, 12 hours, 1 day, 2 days, 3 days, 4 days, 5 days, 6 days, 7 days, 8 days, 9 days, 10 days, 11 days, 12 days, 13 days 14 days, or a month.

In some embodiments, the resting phase 130 is followed by a separation 140 to remove solids and/or microbes from the cell bath mixture. In some embodiments, the separation 140 involves centrifuging or filtering the cell bath mixture to remove large particulates. In some embodiments, the separation 140 involves removing bacteria, viruses, and/or fungi from the cell bath liquid, optionally by filtration. In embodiments, filtration of the cell bath mixture yields a filtered liquid that is called a biocontrol agent, which comprises chemicals that in various embodiments are effective in controlling growth of a fungal pathogen (e.g., Botrytis cinerea, Colletotrichum acutatum, Fusarium oxysporum sp. fragariae, Macrophomina phaseolina, Phytophthora cactorum, Pythium uncinulatum, Rhizoctonia solani, Sclerotinia sclerotiorum, Sclerotium cepivorum, Sclerotinia minor, or Verticillium dahliae). In embodiments, the method may further comprise concentrating the biocontrol agent by removing some or all water from the biocontrol agent to yield biocontrol agent that is concentrated and in a solid or liquid form.

In embodiments, the separation 140 involves filtration (e.g., membrane filtration), sedimentation or settling, centrifugation, or coagulation. In embodiments, the filter used for filtration contains granular media (e.g., sand, gravel, diatomaceous earth, or coal), vegetable or animal media (e.g., sponge, cotton, or charcoal), fabric, paper, canvas, a membrane, or a porous ceramic. In embodiments, the filter is a bucket filter, a barrel filter, a drum filter, or a roughing filter.

In some embodiments, to obtain a desired assembly of microbial taxa, the inoculum may be derived from a variety of non-limiting starting materials, optionally selected to introduce desired microbial taxa to the cell bath mixture. In some embodiments, the inoculum contains bacteria, fungi, and/or archaea. In some embodiments, the inoculum contains a gram-negative bacterium. In some embodiments, the inoculum contains a gram-positive bacterium. In some embodiments, the inoculum may include species from the phyla Acidobacteria, Actinobacteria, Bacteroidetes, Chlamydiae, Chloroflexi, Cyanobacteria, Deinococcus-Thermus, Firmicutes, Gemmatimonadetes, Hydrogenedentes, Nitrospirae, Parcubacteria, Planctomycetes, Proteobacteria, Saccharibacteria, Spirochaetes, Tenericutes, Thaumarchaeota, Verrucomicrobia. In embodiments the inoculum contains a relative abundance of prokaryotic phyla, as measured by 16S rRNA gene sequencing, of at least about 10%, 15%, 20%, 25%, 30%, 35%, 40%, 45%, 50%, 55%, 60%, 65%, or 70% of bacteria selected from the group consisting of Proteobacteria, Firmicutes, and Actinobacteria. In embodiments, about or at least about 25%, 50%, 25% or 80% (relative abundance) of the prokaryotic cells in the cell bath mixture are Firmicutes and/or Gammaproteobacteria during the aeration phase and/or the resting phase. In embodiments, the cell bath mixture comprises a prokaryotic species relative abundance of Bacilli, Clostridia, and/or Gammaproteobacteria of about or of at least about 20%, 30%, 25%, 40%, 45%, 50%, 55%, or 60% during the aeration and/or resting phase(s). In embodiments, when ranked by relative abundance, the top 5 prokaryotic taxa represented in the cell bath mixture includes Bacillus, Clostridium, and/or Leuconostoc during the resting phase. In embodiments, when ranked by relative abundance, the top taxon represented in the cell bath mixture is Bacillus during the aeration phase. Any of various known methods can be used to measure relative abundance of microbes in the cell bath mixtures, such as those described in Tkacz, et al, “Absolute quantitation of microbiota abundance in environmental samples”, Microbiome, 6, Article No.: 110 (2018) or in Suominen, et al., “A diverse uncultivated microbial community is responsible for organic matter degradation in the Black Sea sulphidic zone”, Environmental Microbiology, (2019) doi:10.1111/1462-2920.14902.

In some embodiments, the inoculum forms part of or is prepared using a soil-compost mixture. In some embodiments, the soil-compost mixture contains topsoil. In some embodiments, the soil-compost mixture contains humic compost. In some embodiments, the soil-compost mixture contains an earthworm compost. In some embodiments, the cell bath mixture contains kelp, a fish suspension, feather meal, rock powder, mycorrhizal fungi, amino acids, and/or trace minerals.

In some embodiments, the inoculum and/or cell bath mixture may contain species from one or more of the classes Acidimicrobiia, Alphaproteobacteria, Anaerolinease, Bacilli, Betaproteobacteria, Clostridia, Deltaproteobacteria, Flavobacteriia, Gammaproteobacteria, Nitriliruptoria, Opitutae, Sphingobacteriia, and Thermomicrobia. In some embodiments, the inoculum and/or cell bath mixture may contain species from one or more of the orders Bifidobacteriales, Cytophagia, and Holophagae, Rhodospirillales. In some embodiments, the inoculum and/or cell bath mixture contains species from one or more of the genuses Arthrobacter, Caldilineae, Enterobacter, Leuconostoc, Novosphingobium, Pseudomonas, and Sporolactobacillus

In some embodiments, the soil-compost mixture contains about or less than 5% (w/w) moisture, 10% (w/w) moisture, 15% (w/w) moisture, 20% (w/w) moisture, or 25% (w/w) moisture. In some embodiments, the soil-compost mixture contains greater than about 5% (w/w) moisture, 10% (w/w) moisture, 15% (w/w) moisture, 20% (w/w) moisture, or 25% (w/w) moisture.

In some embodiments, the soil-compost mixture contains about or at least about 40% (w/w) topsoil, 45% (w/w) topsoil, 50% (w/w) topsoil, 55% (w/w) topsoil, 60% (w/w) topsoil, 65% (w/w) topsoil, 70% (w/w) topsoil, 75% (w/w) topsoil, 80% (w/w) topsoil, 85% (w/w) topsoil, 90% (w/w) topsoil, or 95% (w/w) topsoil. In some embodiments, the soil-compost mixture contains no more than about 40% (w/w) topsoil, 45% (w/w) topsoil, 50% (w/w) topsoil, 55% (w/w) topsoil, 60% (w/w) topsoil, 65% (w/w) topsoil, 70% (w/w) topsoil, 75% (w/w) topsoil, 80% (w/w) topsoil, 85% (w/w) topsoil, 90% (w/w) topsoil, or 95% (w/w) topsoil. In some embodiments, the soil-compost mixture contains about or at least about 5% (w/w) humic compost, 10% (w/w) humic compost, 15% (w/w) humic compost, 20% (w/w) humic compost, 25% (w/w) humic compost, 30% (w/w) humic compost, 25% (w/w) humic compost about 40% (w/w) humic compost. In some embodiments, the soil-compost mixture comprises about 1% (w/w) earthworm compost, about 2% (w/w) earthworm compost, about 3% (w/w) earthworm compost, about 4% (w/w) earthworm compost, about 5% (w/w) earthworm compost, about 6% (w/w) earthworm compost, about 7% (w/w) earthworm compost, about 8% (w/w) earthworm compost, about 9% (w/w) earthworm compost, about 10% (w/w) earthworm compost, or ranges including and/or spanning the aforementioned values.

In some embodiments, in the cell bath mixture, the volumetric ratio of soil-compost solids containing the inoculum-to-water at startup is about or less than about 1:1, 1:2, 1.3, 1:4, 1:5, 1:6, 1:7, 1:8, 1.9, 1:10, 1.11, 1:12, 1:13, 1:14, 1.15, 1:16, 1:17, 1:18, 1:19, 1:20, 1:25, 1:30, 1:35, 1:40, 1:45, 1:50, 1:75, 1:100, 1:250, 1:500, 1:750, 1:1,000, 1:2,500, 1:5,000, or 1:10,000. In some embodiments, the cell bath mixture comprises about or at least about 1% (w/v), 2% (w/v), 3% (w/v), 4% (w/v), 4.5% (w/v), 5% (w/v), 5.5% (w/v), 6% (w/v), 6.5% (w/v), 7% (w/v), 7.5% (w/v), 8% (w/v), 8.5% (w/v), 9% (w/v), 9.5% (w/v), 10% (w/v), 15% (w/v), 20% (w/v), or 25% (w/v) of solids, optionally where the solids are insoluble in water (e.g., an insoluble soil-compost composition). In some embodiments, the cell bath mixture comprises no more than about 1% (w/v), 2% (w/v), 3% (w/v), 4% (w/v), 4.5% (w/v), 5% (w/v), 5.5% (w/v), 6% (w/v), 6.5% (w/v), 7% (w/v), 7.5% (w/v), 8% (w/v), 8.5% (w/v), 9% (w/v), 9.5% (w/v), 10% (w/v), 15% (w/v), 20% (w/v), or 25% (w/v) of solids, optionally where the solids are insoluble in water (e.g., an insoluble soil-compost composition).

In some embodiments, the carbon/energy source added to the cell bath mixture contains ethanol and/or one or more sugars. In some embodiments, the carbon/energy source contains a sugar syrup or molasses. In some embodiments, the sugar syrup comprises corn syrup (e.g., high fructose corn syrup or a corn syrup mixture containing high fructose corn syrup), and a salt (e.g., NaCl). In some embodiments, the carbon/energy source contains a sucrose sugar. In some embodiments, the sucrose sugar is a 1:1 ratio of glucose: fructose. In some embodiments, the concentration of a carbon/energy source in the microbial media is about or at least about 5 mM, 10 mM, 15 mM, 20 mM, 25 mM, 30 mM, 35 mM, 40 mM, 45 mM, 50 mM, 60 mM, 70 mM, 80 mM, 90 mM, or 100 mM. In some embodiments, the concentration of a carbon/energy source in the microbial media is no more than about 5 mM, 10 mM, 15 mM, 20 mM, 25 mM, 30 mM, 35 mM, 40 mM, 45 mM, 50 mM, 60 mM, 70 mM, 80 mM, 90 mM, or 100 mM. In some embodiments, the carbon/energy source concentration the cell bath mixture is about or at least about 4% (w/w), 4.5% (w/w), 5% (w/w), 5.5% (w/w), 6% (w/w), 6.5% (w/w), 7% (w/w), 7.5% (w/w), 8% (w/w), 8.5% (w/w), 9% (w/w), 9.5% (w/w), 10% (w/w). In some embodiments, the carbon/energy source concentration in the cell bath mixture is less than about 4% (w/w), 4.5% (w/w), 5% (w/w), 5.5% (w/w), 6% (w/w), 6.5% (w/w), 7% (w/w), 7.5% (w/w), 8% (w/w), 8.5% (w/w), 9% (w/w), 9.5% (w/w), 10% (w/w).

In embodiments, the carbon/energy source is added to the cell bath mixture once. In embodiments, the carbon/energy source is added to the cell bath mixture multiple times. In embodiments, the carbon/energy source is added to the cell bath mixture every 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, or a combination thereof over a total duration of 1 hour, 2 hours, 3 hours, 4 hours, 5 hours, 6 hours, 7 hours, 8 hours, 9 hours, 10 hours, 11 hours, 12 hours, 1 day, 2 days, 3 days, 4 days, 5 days, 6 days, 7 days, or a combination thereof. In some embodiments, the carbon/energy source is added to the cell bath at an independently selected frequency for an independently selected duration more than once during preparation of a biocontrol agent. In embodiments, the carbon/energy source is added during the aerobic phase and/or during the resting phase.

In some embodiments, the aeration phase involves bubbling a gas (e.g., air) into the cell bath mixture. In some embodiments, the volumetric flow rate of gas is about or at least about 0.5 ft³/min, 1 ft³/min, 2 ft³/min, 3 ft³/min, 4 ft³/min, 5 ft³/min, 6 ft³/min, 7 ft³/min, 8 ft³/min, 9 ft³/min, to ft³/min, 12 ft³/min, 14 ft³/min, 16 ft³/min, 18 ft³/min, 20 ft³/min, 22 ft³/min, 24 ft³/min, 26 ft³/min, 28 ft³/min, or 30 ft³/min, where the volume of the gas is calculated at 1 atm and 25° C. In some embodiments, the volumetric flow rate of gas is less than about 6 ft³/min, 7 ft³/min, 8 ft³/min, 9 ft³/min, 10 ft³/min, 12 ft³/min, 14 ft³/min, 16 ft³/min, 18 ft³/min, 20 ft³/min, 22 ft³/min, 24 ft³/min, 26 ft³/min, 28 ft³/min, or 30 ft³/min, where the volume of the gas is calculated at 1 atm and 25° C.

In some embodiments, the cell bath mixture temperature during startup, aeration phase, and/or resting phase is about or at least about 15° C., 16° C., 17° C., 18° C., 19° C., 20° C., 21° C., 22° C., 23° C., 24° C., 25° C., 30° C., 31° C., 32° C., 33° C., 34° C., or 35° C. In some embodiments, the cell bath mixture temperature during startup, aeration phase, and/or resting phase is less than about 15° C., 16° C., 17° C., 18° C., 19° C., 20° C., 21° C., 22° C., 23° C., 24° C., 25° C., 30° C., 31° C., 32° C., 33° C., 34° C., or 35° C.

In some embodiments, the pH of the cell bath mixture during startup, aeration phase, and/or resting phase is about or at least about 4, 4.3, 4.6, 4.9, 5.2, 5.5, 5.8, 6.1, 6.4, 6.7, 7.0, 7.3, 7.6, 7.9, or 8.2. In some embodiments, the pH of the cell bath mixture during startup, aeration phase, and/or resting phase is no more than about 4, 4.3, 4.6, 4.9, 5.2, 5.5, 5.8, 6.1, 6.4, 6.7, 7.0, 7.3, 7.6, 7.9, or 8.2.

In some embodiments, the electrical conductivity of the cell bath mixture during startup, aeration phase, and/or resting phase is about or at least about 0 µS m⁻¹, 50 µS m⁻¹, 100 µS m⁻¹, 150 µS m⁻¹, 200 µS m⁻¹, 250 µS m⁻¹, 300 µS m⁻¹, 350 µS m⁻¹, 400 µS m⁻¹, 450 µS m⁻¹, 500 µS m⁻¹, 550 µS m⁻¹, 600 µS m⁻¹, 650 µS m⁻¹, 700 µS m⁻¹, 750 µS m⁻¹, 800 µS m⁻¹, 850 µS m⁻¹, 900 µS m⁻¹, 950 µS m⁻¹, 1000 µS m⁻¹, 1250 µS m⁻¹, 1500 µS m⁻¹, 1750 µS m⁻¹, 2000 µS m⁻¹, 2500 µS m⁻¹, or 3000 µS m⁻¹. In some embodiments, the electrical conductivity of the cell bath mixture during startup, aeration phase, and/or resting phase is no more than about 0 µS m⁻¹, 50 µS m⁻¹, 100 µS m⁻¹, 150 µS m⁻¹, 200 µS m⁻¹, 250 µS m⁻¹, 300 µS m⁻¹, 350 µS m⁻¹, 400 µS m⁻¹, 450 µS m⁻¹, 500 µS m⁻¹, 550 µS m⁻ ¹, 600 µS m⁻¹, 650 µS m⁻¹, 700 µS m⁻¹, 750 µS m⁻¹, 800 µS m⁻¹, 850 µS m⁻¹, 900 µS m⁻¹, 950 µS m⁻¹, 1000 µS m⁻¹, 1250 µS m⁻¹, 1500 µS m⁻¹, 1750 µS m⁻¹, 2000 µS m⁻¹, 2500 µS m⁻¹, or 3000 µS m⁻¹.

In embodiments, the filter used for the filtration has a nominal pore size of about or of less than about 0.5 µm, 0.45 µm, 0.4 µm, 0.35 µm, 0.3 µm, 0.25 µm, 0.2 µm, 0.15 µm, 0.1 µm, 0.05 µm, or 0.025 µm.

Some embodiments provided herein relate to systems for producing compositions (e.g., biocontrol agents) described herein. In some embodiments, the systems contains a fluid treatment apparatus for preparation of sterile and/or ion-free water (e.g., a water distillation system) In embodiments, the system comprises a fermentation vessel (e.g., an open-top water storage tank). Appropriate dimensions of the fermentation vessel and appropriate materials for the fermentation vessel will be apparent to one of skill in the art. For example, the materials of the vessel may be selected to be resistant to corrosion. In embodiments the fermentation vessel includes a mixing blade or propeller for agitating the cell bath mixture fluid. The systems of the invention can include input conduits, output conduits, sampling valves, switches, pumps lines, hoses, housing, motors, fans, propellers, impellers, agitators, aerators, over-flow containers, thermometers, insulation, actuators, filters, concentrators, and the like. In various embodiments, equipment and methods used in industrial fermentations may be employed in the methods and systems of the invention, such as those described in The Encyclopedia of Food Microbiology (Second Edition), 2014 (ISBN 978-0-12-227070-3) or in Comprehensive Biotechnology (Second Edition), 2011 (ISBN: 978-0-08-088504-9).

The systems of the invention can be manually operated or automated. The systems of the invention in various embodiments contain computer processors, circuits, sensors, monitors, feedback loops (e.g., real-time feedback loops), pumps, actuators, switches, or any combination thereof. In some embodiments, the automated systems continuously or periodically (e.g., about every 1 min, 5 min, 10 min, 15 min, 30 min, 60 min, or 120 min) regulate, monitor, and/or alter conditions in the cell bath mixture.

### Compositions

The invention provides compositions used for inhibiting the growth and/or survival of a plant (e.g., a crop plant, tree, ornamental plant, turf, lettuce, or allium plant) fungal pathogen (e.g., Botrytis cinerea, Colletotrichum acutatum, Fusarium oxysporum sp. fragariae, Macrophomina phaseolina, Phytophthora cactorum, Pythium uncinulatum, Rhizoctonia solani, Sclerotinia sclerotiorum, Sclerotium cepivorum, Sclerolinia minor, or Verticillium dahliae). In embodiments, the compositions are produced by the methods provided herein. In embodiments, the compositions contain components of a composition produced by the methods provided herein. Aspects provided herein relate to compositions used for mitigating, controlling, or reducing harmful effects caused by pathogens.

In embodiments, the fungal pathogen belongs to a genus selected from Botrytis, Colletotrichum, Fusarium, Macrophomina, Phytophthora, Pythium, Rhizoctonia, Sclerotinia, Sclerotiniaceae, Sclerotium, and Verticillium. In embodiments, the plant pathogen is Botrytis cinerea, Colletotrichum acutatum, Fusarium oxysporum sp. fragariae, Macrophomina phaseolina, Phytophthora cactorum, Pythium uncinulatum, Rhizoctonia solani, Sclerotinia sclerotiorum, Sclerotium cepivorum, Sclerotinia minor, or Verticillium dahliae.

In some embodiments, the composition may include one or more microbial metabolites from a microbial cell bath mixture. In some embodiments, the microbial cell bath mixture comprises species of the phyla Acidobacteria, Actinobacteria, Bacteroidetes, Chlamydiae, Chloroflexi, Cyanobacteria, Deinococcus-Thermus, Firmicutes, Gemmatimonadetes, Hydrogenedentes, Nitrospirae, Parcubacteria, Planctomycetes, Proteobacteria, Saccharibacteria, Spirochaetes, Tenericutes, Thaumarchaeota, Verrucomicrobia.

In various embodiments, a composition of the present invention is characterized as having a particular concentration of dissolved solids. In embodiments, the concentration of the dissolved solids is about or at least about 50 ppm, 75 ppm, 100 ppm, 125 ppm, 150 ppm, 175 ppm, 200 ppm, 300 ppm, 400 ppm, 500 ppm, 600 ppm, 700 ppm, 800 ppm, 900 ppm, 1,000 ppm, 1,100 ppm, 1,200 ppm, 1,300 ppm, 1,400 ppm, 1,500 ppm, 1,600 ppm, 1,700 ppm, 1,800 ppm, 1,900 ppm, 2,000 ppm, 2,500 ppm, 3,000 ppm, 3,500 ppm, 5,000 ppm, 5,500 ppm, 10,000 ppm, 15,000 ppm, 20,000 ppm, 50,000 ppm, 100,000 ppm, 200,000 ppm, 300,000 ppm, 400,000 ppm, or 500,000 ppm. In some embodiments, the concentration of the dissolved solids is not greater than about 50 ppm, 75 ppm, 100 ppm, 125 ppm, 150 ppm, 175 ppm, 200 ppm, 300 ppm, 400 ppm, 500 ppm, 600 ppm, 700 ppm, 800 ppm, 900 ppm, 1,000 ppm, 1,100 ppm, 1,200 ppm, 1,300 ppm, 1,400 ppm, 1,500 ppm, 1,600 ppm, 1,700 ppm, 1,800 ppm, 1,900 ppm, 2,000 ppm, 2,500 ppm, 3,000 ppm, 3,500 ppm, 5,000 ppm, 5,500 ppm, 10,000 ppm, 15,000 ppm, 20,000 ppm, 50,000 ppm, 100,000 ppm, 200,000 ppm, 300,000 ppm, 400,000 ppm, or 500,000 ppm.

The compositions may comprise agriculturally acceptable carriers and/or additives. Non-limiting examples of carriers and/or additives include extenders, solvents, diluents, dyes, wetters, dispersants, emulsifiers, antifoaming agents, nutrients, preservatives, secondary thickeners, adhesives, and/or water. Formulations of the present invention may include agriculturally acceptable carriers, which are inert formulation ingredients added to formulations to improve recovery, efficacy, or physical properties and/or to aid in packaging and administration. Carriers may include anti-caking agents, anti-oxidation agents, bulking agents, and/or protectants. Examples of useful carriers include polysaccharides (starches, maltodextrins, methylcelluloses, proteins, such as whey protein, peptides, gums), sugars (lactose, trehalose, sucrose), lipids (lecithin, vegetable oils, mineral oils), salts (sodium chloride, calcium carbonate, sodium citrate), silicates (clays, amorphous silica, fumed/precipitated silicas, silicate salts), waxes, oils, alcohol and surfactants.

In some embodiments, the microbial metabolites may include micronutrients and/or macronutrients. In some embodiments, the composition may comprise micronutrients and/or macronutrients that, promotes improved seedling emergence or survival. In some embodiments, the composition may comprise micronutrients and/or macronutrients that promotes increased yields. In some embodiments, the composition may comprise micronutrients and/or macronutrients that reduce the prevalence of an undesired pathogens, such as, bacteria, virus or fungi in the soil. In some embodiments, the micronutrients and macronutrients are selected from a group selected from lithium, sodium, ammonium, magnesium, potassium, calcium, strontium, barium, fluoride, chlorine, nitrite, bromide, nitrate, sulfate, phosphate, lactate, acetate, propionate, formate, methanesulfonate, succinate maleate, and oxalate. In some embodiments, the micronutrients range from 0.01 ppm to about 1000 ppm. In some embodiments, the micronutrients range from about 0.01, 0.05, 0.1, 0.15, 2, 2.5, 3, 5, 7, 10, 15, 20, 25, 50, 75, 100, 125, 150, 200, 300, 400, 500, 700, 900, 1000 ppm, or in an amount within a range defined by any two of the aforementioned values.

Further non-limiting examples of carriers include a natural or synthetic, organic or inorganic substance which is mixed or combined with a biocontrol agent for better applicability, in particular for application to plants or plant parts, soils, or seeds. The support or carrier, which may be solid or liquid, is generally inert and should be suitable for use in agriculture. Suitable solid or liquid carriers/supports include for example ammonium salts and natural ground minerals, such as kaolins, clays, talc, chalk, quartz, attapulgite, montmorillonite or diatomaceous earth, and ground synthetic minerals, such as finely divided silica, alumina and natural or synthetic silicates, resins, waxes, solid fertilizers, water, alcohols, especially butanol, organic solvents, mineral oils and vegetable oils, and also derivatives and various combinations thereof. It is also possible to use mixtures of such supports or carriers. Solid supports/carriers suitable for granules are: for example crushed and fractionated natural minerals, such as calcite, marble, pumice, sepiolite, dolomite, and also synthetic granules of inorganic and organic meals and also granules of organic material, such as sawdust, coconut shells, maize cobs and tobacco stalks. Suitable liquefied gaseous extenders or carriers are liquids which are gaseous at ambient temperature and under atmospheric pressure, for example aerosol propellants, such as butane, propane, nitrogen and carbon dioxide. Tackifiers, such as carboxymethylcellulose and natural and synthetic polymers in the form of powders, granules and latices, such as gum arabic, polyvinyl alcohol, polyvinyl acetate, or else natural phospholipids, such as cephalins and lecithins and synthetic phospholipids can be used in the formulations. Other possible additives are mineral and vegetable oils and waxes, optionally modified. If the extender used is water, it is also possible for example, to use organic solvents as auxiliary solvents. Suitable liquid solvents are essentially: aromatic compounds, such as xylene, toluene or alkylnaphthalenes, chlorinated aromatic compounds or chlorinated aliphatic hydrocarbons, such as chlorobenzenes, chloroethylenes or methylene chloride, aliphatic hydrocarbons, such as cyclohexane or paraffins, for example mineral oil fractions, mineral and vegetable oils, alcohols, such as butanol or glycol, and also ethers and esters thereof, ketones, such as acetone, methyl ethyl ketone, methyl isobutyl ketone or cyclohexanone, strongly polar solvents, such as dimethylformamide and dimethyl sulphoxide, and also water.

In some embodiments, the composition may include components that facilitate the application of the composition to a plant or soil. The application of a composition of the invention to soil may be performed by drenching, incorporation into soil, or by droplet application. The compositions may also be applied directly to plant roots or seeds (e.g., via immersion, dusting, or spraying). To assist in the application, the compositions can be in the form of liquid solutions, emulsions, wettable powders, suspensions, powders, dusts, pastes, soluble powders, granules, or suspension-emulsion concentrates.

In some embodiments, the composition may be a sterile liquid solution. In some embodiments, the composition may contain a liquid diluent or solvent (e.g., water). A non-limiting example of a diluent is an aqueous solution that is compatible with plant, soil, aquaculture, or livestock application, such that the composition does not adversely affect the growth of plants, aquatic life, or livestock. The carrier may be a liquid. The carrier may improve the stability, handling, storage, shipment, or application properties of the composition.

In some embodiments, the compositions further include a surfactant. In some embodiments, the surfactant includes glycerol, alkylbenzenesulfonate, ammonium lauryl sulfate, sodium lauryl sulfate (SLS), sodium dodecyl sulfate (SDS), sodium laureth sulfate, sodium lauryl ether sulfate (SLES), sodium myreth sulfate, dioctyl sodium sulfosuccinate, perfluorooctane sulfonate, perfluorobutanesulfonate, alkyl- aryl ether phosphates, alkyl ether phosphates, sodium stearate, sodium lauroyl sarcosinate, perfluorononanoate, and perfluorooctanoate. In some embodiments, the compositions include an emulsifier present in an amount of ranging from about 0.001% to about 10%, such as 0.001, 0.005, 0.01, 0.05, 0.1, 0.5, 1, 1.5, 2, 2.5, 3, 3.5, 4, 4.5, 5, 5.5, 6, 6.5, 7, 7.5, 8, 8.5, 9, 9.5, or 10%, or in an amount within a range defined by any two of the aforementioned values.

In some embodiments, the surfactant comprises an emulsifier, a dispersing agent or a wetting agent of ionic or non-ionic type or a mixture of such surfactants. Further non-limiting examples of surfactants include polyacrylic acid salts, lignosulphonic acid salts, phenolsulphonic or naphthalenesulphonic acid salts, polycondensates of ethylene oxide with fatty alcohols or with fatty acids or with fatty amines, substituted phenols (in particular alkylphenols or arylphenols), salts of sulphosuccinic acid esters, taurine derivatives (in particular alkyl taurates), phosphoric esters of polyoxyethylated alcohols or phenols, fatty acid esters of polyols, and derivatives of the above compounds containing sulphate, sulphonate and phosphate functions.

Additional components may also be included in the compositions, as non-limiting examples, protective colloids, adhesives, nutrients, thickeners, thixotropic agents, penetration agents, stabilizers, sequestering agents.

In some embodiments, the compositions comprise colorants, such as inorganic pigments (e.g., iron oxide, titanium oxide, and Prussian blue), and organic dyes (e.g., alizarin dyes, and azo dyes) and metal phthalocyanine dyes.

In some embodiments, the composition is formulated as a sterile liquid media, a solution, a spray, a mist, a seed coating, an electrostatically charged seed powder, a powder, a powder-like substance, or a freeze-dried powder.

In some embodiments, additional components may be included in compositions, as non-limiting examples, such as benzoids, pyrazines, alcohols, ketones, volatile fatty acids, volatile organic compounds, sulfides and/ or alkenes.

In some embodiments, the composition may be formulated as a seed coating. In some embodiments, the composition may be a conglomerate mixture with additional nutrients used to coat a plant seed. In some embodiments, the composition protects the plant seed from harmful pathogens, such as fungi, during storage. In some embodiments, the composition increases germination rates, increases seedling survival, and/or increases crop yields.

In some embodiments, the composition may be formulated for application to a crop, a plant a tree, turf, or soil by spraying, misting, soaking, watering, soil drenching, crop-dusting, or otherwise applying the composition to the soil, plants, the portion of the plants, or components of the plants. In some embodiments, the composition is applied to the plant itself, such as to the leaves, stem, trunk, stalk, flowers, branches, fruits, roots, shoots, buds, rhizome, seeds, or other portions of the plant, or it is applied to the soil in which or around which the plant is being cultivated. In some embodiments, the composition is formulated as a solution that is applied to the plant or to plant parts, such as applied to harvested seeds, leaves, stem, trunk, stalk, flowers, branches, fruits, roots, shoots, buds, rhizome, or other portions of the plant, or to the soil in which or around which the plant is being cultivated. In some embodiments, the composition is applied to turf grass. In some embodiments, the composition is freeze-dried or otherwise reduced to a solid or powder through an evaporative process. In some embodiments, the composition is formulated together with a fertilizer or micro-nutrient for application to a plant or soil. Such fertilizers or nutrients may include, for example, trace minerals, phosphorus, potassium, sulfur, manganese, magnesium, calcium, and/or any one or more of a trace element. In some embodiments, the composition is formulated as a concentrated composition that may be diluted prior to application. For example, the composition may be formulated as a liquid concentrate that may be diluted with a solution, such as with water, or it may be formulated as a solid, such as a powder, for dissolution in a solution, such as water. In some embodiments, the composition may be formulated as a ready-to-use composition. For example, the composition may be formulated as a solution that includes the appropriate concentrations of component parts for direct application to a plant or may be formulated as a solid for direct application to a plant.

In any of the embodiments of the compositions provided herein, formulations may be developed as adjuvants to be applied concurrently with existing commercial products to enable and/or enhance their effectiveness.

In any of the embodiments of the compositions provided herein, the compositions may be non-toxic and include component parts that exhibit no toxic effects to humans, to the soil or plant that is being treated, or to the environment, including no toxicity to groundwater, flora, or fauna. Components suitable for use in any of the embodiments of the compositions provided herein can result in improved agricultural health, including improved plant health and/or improved crop production, or improved aquaculture or livestock health. Furthermore, embodiments of the compositions provided herein enable ease in application of the compositions.

Compositions according to the present invention can be used in various forms such as aerosol dispenser, capsule suspension, cold fogging concentrate, dustable powder, emulsifiable concentrate, emulsion oil in water, emulsion water in oil, encapsulated granule, fine granule, flowable concentrate for seed treatment, gas (under pressure), gas generating product, granule, hot fogging concentrate, macrogranule, microgranule, oil dispersible powder, oil miscible flowable concentrate, oil miscible liquid, paste, plant rodlet, powder for dry seed treatment, soluble concentrate, soluble powder, liquid solution, suspension concentrate (flowable concentrate), water dispersible granules or tablets, water dispersible powder for slurry treatment, water soluble granules or tablets, water soluble powder, and wettable powder.

These compositions include not only compositions which are ready to be applied to a plant (e.g., crop plant, tree, ornamental plant, turf, lettuce, or an Allium plant), seed, or soil to be treated by means of a suitable device, such as a spraying or dusting device, but also concentrated commercial compositions (i.e., concentrates) which must be diluted before they are applied to a soil or plant.

In some embodiments, the composition is a soil or a potting soil. The soil or potting soil may be disposed in, to provide non-limiting examples, a planter, a pot, a bag, or a sealed bag.

### Methods of Delivery

In some embodiments, the methods include treating soil or a plant (e.g., crop plant, tree, ornamental plant, turf, lettuce, or an Allium plant) having a fungal disease with the compositions described herein. In embodiments, the fungal disease is associated with a plant pathogen (e.g., Botrytis cinerea, Colletotrichum acutatum, Fusarium oxysporum sp. fragariae, Macrophomina phaseolina, Phytophthora cactorum, Pythium uncinulatum, Rhizoctonia solani, Sclerotinia sclerotiorum, Sclerotium cepivorum, Scleratinia minor, or Verticillium dahliae). In some embodiments, the pathogen is resistant to pesticides in common use.

In embodiments, the fungal pathogen belongs to a genus selected from Botrytis, Colletotrichum, Fusarium, Macrophomina, Phytophthora, Pythium, Rhizoctonia, Sclerotinia, Sclerotiniaceae, Sclerotium, and Verticillium. In embodiments, the plant pathogen is Botrytis cinerea, Colletotrichum acutatum, Fusarium oxysporum sp. fragariae, Macrophomina phaseolina, Phytophthora cactorum, Pythium uncinulatum, Rhizoctonia solani, Sclerotinia sclerotiorum, Sclerotium cepivorum, Sclerotinia minor, or Verticillium dahliae.

The precise amount of a composition of the present invention to be applied to a particular plant or soil in accordance with the invention will depend upon the sensitivities of the particular plant, the method of application, and field conditions such as the quality of the soil. All of these factors can be taken into consideration by one skilled in the art to determine an optimal amount of biocontrol agent to apply to a plant or soil for a particular application. The compositions are applied to a plant or soil in an amount effective to control (e.g., inhibit growth or survival) a pathogen.

In various embodiments, a composition of the present invention is applied to a soil, crop plant, tree, turf, or ornamental plant until a target concentration of dissolved solids originating from the composition is achieved in the soil and/or on the plant. In embodiments, the target concentration of the dissolved solids in the soil is about or at least about 50 ppm, 75 ppm, 100 ppm, 125 ppm, 150 ppm, 175 ppm, 200 ppm, 300 ppm, 400 ppm, 500 ppm, 600 ppm, 700 ppm, 800 ppm, 900 ppm, 1,000 ppm, 1,100 ppm, 1,200 ppm, 1,300 ppm, 1,400 ppm, 1,500 ppm, 1,600 ppm, 1,700 ppm, 1,800 ppm, 1,900 ppm, 2,000 ppm, 2,500 ppm, 3,000 ppm, 3,500 ppm, 5,000 ppm, or 5,500 ppm. In some embodiments, the target concentration of the dissolved solids in the soil and/or on the plant is not greater than about 50 ppm, 75 ppm, 100 ppm, 125 ppm, 150 ppm, 175 ppm, 200 ppm, 300 ppm, 400 ppm, 500 ppm, 600 ppm, 700 ppm, 800 ppm, 900 ppm, 1,000 ppm, 1,100 ppm, 1,200 ppm, 1,300 ppm, 1,400 ppm, 1,500 ppm, 1,600 ppm, 1,700 ppm, 1,800 ppm, 1,900 ppm, 2,000 ppm, 2,500 ppm, 3,000 ppm, 3,500 ppm, 5,000 ppm, or 5,500 ppm.

In embodiments, a volume of a composition of the present invention is applied per a unit area of an agricultural field or soil. In embodiments, the volume of the composition applied per acre of a field or soil is about or at least about 500 gal, 750 gal, 1000 gal, 1,250 gal, 1,500 gal, 1,750 gal, 2,000 gal, 2,250 gal, 2,500 gal, 2,750 gal, 3,000 gal, or 3,500 gal. In embodiments, the volume of the composition applied per acre of a field is no more than about 500 gal, 750 gal, 1000 gal, 1,250 gal, 1,500 gal, 1,750 gal, 2,000 gal, 2,250 gal, 2,500 gal, 2,750 gal, 3,000 gal, or 3,500 gal.

In embodiments the composition is applied to a soil and/or plant multiple times. In embodiments, the soil and/or plant is contacted with the composition about or at least about 1, 2, 3, 4, 5, 6, 7, 8, 9, 10, 11, 12, 13, 14, 15, 16, 17, 18, 19, or 20 times. In various embodiments, each contacting is spaced from the previous contacting by a time interval individually ranging from about or at least about 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days 24 days, or 25 days. In various embodiments, the composition is applied to the soil before the time of planting by a time interval ranging from about or at least about 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days 24 days, or 25 days before planting. In embodiments, the composition is applied to the soil and/or plant at time of planting. In embodiments, the composition is applied to the soil and/or plant at 10 days, 14 days, 28 days, and 42 days after planting. In embodiments, the composition is applied by spray or drip application. In embodiments, the composition is applied at 14 days, 30 days, 36 days, and 42 days post-planting. In embodiments, a last application of the composition is by drip application.

In embodiments, application of the composition does not adversely affect the vigor of a plant. In embodiments, the application of the composition is not toxic to a plant.

In some embodiments, the compositions are applied to a plant or soil at a time of planting or prior to the time of planting. The compositions can also be applied once plants are established within the soil. The compositions can be applied to seeds, reproductive vegetative material, seedlings, and/or established plants.

In some embodiments, the soil or plant is treated for a potential or actual fungal pathogenic disease. The soil can be outside or inside (e.g., in a greenhouse or other enclosure). The plant could be an ornamental, a crop, a tree, a turf, or an aquaculture plant. The soil can be soil used for the production of any agricultural or horticultural product, such as cereals, vegetables, fruits, nuts, beans, seeds, herbs, spices, fungi, ornamental plants (e.g., flowers, bushes, turf, and trees), industrial plants, and/or plants grown for feed. In some embodiments, the plant or soil exhibits industrial, commercial, recreational, or aesthetic value. In some embodiments, the compositions of the present invention are used to treat a plant. In some embodiments, the plant is a poinsettia, flowers, lupin, grass, alfalfa, trees, or ivy. In some embodiments the plant is a food producing plant. In some embodiments, the plant is a banana, cacao, canola, coffee, bean, cotton, garlic, onion, leek, chive, maize, wheat, rice, corn, leafy greens, potato, tomato, pepper, squash, gourds, cucumber, berry, grape vine or grapes, pome, drupe, citrus, melon, tropical fruit, cotton, nut, soybean, sorghum, cane, cucurbits, onion, aubergine, parsnip, Cannabis (e.g., hemp), herb, tobacco, or pulse plant. The plant can be an Allium plant. The plant can be romaine lettuce or garlic. Non-limiting examples of allium plants include Allium sativum, Allium cepa, Allium chinense, Allium stipitatum, Allium schoenoprasum, Allium tuberosum, Allium fistulosum, and Allium ampeloprasum.

In some embodiments, the methods include applying the composition to a plant or to the soil in which the plant is growing. Applying the composition may be achieved by various means, including, for example, by spraying, sprinklering, drenching, soaking, watering, crop-dusting, misting, high-pressure liquid injection, or otherwise applying the composition to the plants or surrounding soil. The composition can be applied using an irrigation system. In some embodiments, the composition is applied to the plant itself, such as to the leaves, stem, trunk, stalk, flowers, branches, fruits, roots, shoots, buds, rhizome, seeds, or other portions of the plant, or it is applied to the soil in which or around which the plant is being cultivated. In some embodiments, the composition is formulated as a seed coating, and the method includes coating a seed with the composition. In some embodiments, the seed coating is an electrostatic seed coating In some embodiments, the seed coating includes micronutrients. In some embodiments, the seed coating protects the plant seed from harmful pathogens, such as fungi. In some embodiments, the seed coating allows for uniform size of plant seeds for bulk planting techniques. In some embodiments, the seed coating increases germination rates, increases seedling survival, and/or increases crop yields. In some embodiments, the composition is formulated as a powder, and the method includes applying the powder to the plant or to plant parts, such as applied to seeds, leaves, stem, trunk, stalk, flowers, branches, fruits, roots, shoots, buds, rhizome, or other portions of the plant, or to the soil in which or around which the plant is being cultivated. In some embodiments, the composition is formulated together with a fertilizer or nutrient, and the method includes incorporating the composition into the soil through disking or tilling or applying the fertilizer or nutrient to the plant. The compositions of the invention can be applied to a plant seed, to soil within which a plant is growing, to soil in which a plant or seed is about to be planted, to a plant (e.g., plant roots), or to combinations thereof.

### Pathogen Characterization

In some embodiments, the methods of the disclosure include detecting the presence of a pathogenic fungus in soil or on a plant. The method can further include adding a composition of the present invention to the soil or contacting the plant with the composition only if presence of the pathogenic fungus is detected. One of skill in the art will be able to determine a suitable method for determining the presence of a fungal pathogen in soil or on a plant. Non-limiting examples of methods for detecting the presence of a fungal pathogen in soil or on a plant include visual inspection, microscopic techniques, next generation sequencing, DNA microarrays, macroarrays (e.g., membrane-based DNA macroarrays, as described by Lievens, et al, “Fungal plant pathogen detection in plant and soil samples using DNA macroarrays,” Methods Mol. Biol. 835:491-507 (2012), which is incorporated herein by reference in its entirety for all purposes), and PCR. The methods of the present invention can include monitoring effectiveness of a compositions of the present invention in inhibiting, controlling, reducing, or eliminating growth of a plant pathogenic fungus by measuring a titer of the pathogenic fungus in soil or on a plant before, during, and/or after application of the composition to the soil or plant. In some embodiments, a method of the disclosure includes modifying the amount of biocontrol agent applied to a soil or plant to optimize a reduction in titer or growth rate of a pathogenic fungus in the soil or in or on the plant.

In one embodiment, the method of the disclosure includes determining the composition of a microbial community associated with a plant or soil treated by the method. In some embodiments, the composition of the microbial community is determined using techniques familiar to one of skill in the art including, as non-limiting examples, PCR, next generation sequencing, and DNA microarrays. In some embodiments, the composition of the microbial community is determined by sequencing a 16S and/or 18S rRNA gene.

### Kits

This disclosure provides a kit that includes a composition of the present invention for controlling growth of a plant (e.g., a crop plant, tree, ornamental plant, turf, lettuce, or allium plant) fungal pathogen (e.g., Botrytis cinerea, Colletotrichum acutatum, Fusarium oxysporum sp. fragariae, Macrophomina phaseolina, Phytophthora cactorum, Pythium uncinulatum, Rhizoctonia solani, Sclerotinia sclerotiorum, Sclerotium cepivorum, Sclerotinia minor, or Verticillium dahliae) In some embodiments, the kit comprises an applicator. In some embodiments, the kit is a ready-to-use kit, wherein the composition included in the kit is ready to use by the user without further alterations. In some embodiments, the composition is provided in the kit in a container for application to a plant (e.g., a lettuce or an allium plant) or soil. In some embodiments, the container is a spray applicator containing the composition. In some embodiments, the composition is a concentrated liquid, or a solid. In such embodiments, the composition may be added to a liquid, such as water, to dilute the concentrated liquid or to dissolve the solid composition. In some embodiments, the composition is a diluted composition In some embodiments, the spray applicator is configured for industrial, commercial, home-gardener, or recreational purposes. In some embodiments, the kit includes a dispensing apparatus, such as a nozzle, a valve, a sprayer, or any other apparatus capable of dispensing the compositions described herein.

If desired, the kit further contains instructions for using the compositions and/or administering the compositions. In particular embodiments, the instructions include at least one of the following: description of the components of the composition; application amounts and techniques; precautions; warnings; counter-indications; instructions on how to monitor soil organic acid compositions; instructions on how to monitor soil for the presence of a pathogenic fungus; instructions on how to determine composition of a soil microbiome; and/or references. The instructions may be printed directly on components of the kit or provided as a separate sheet, pamphlet, card, or folder supplied with the kit. The instructions can be provided in digital form on a portable data storage medium (e.g., a compact disk or USB drive) or stored remotely on a server that can be accessed remotely.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1. Production of Compositions for Producing Disease Suppression Using a Cell Batch Process

The present example describes a process by which a biocontrol agent was produced by the methods of the invention. The process followed the method outlined in FIG. 1. Two batches of biocontrol agent were prepared as described below.

### Startup

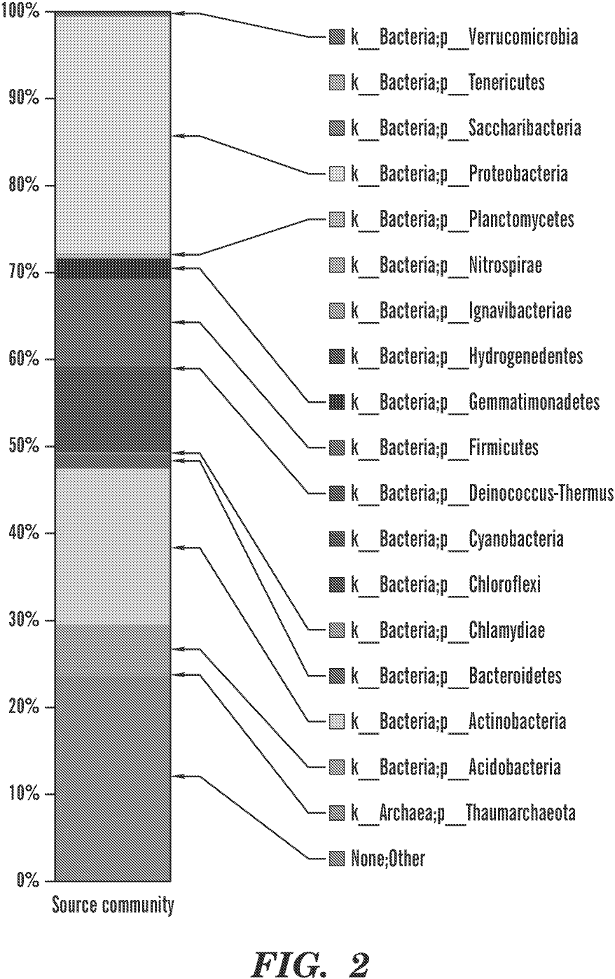

Inoculum preparation: A soil-compost mixture containing a desired abundance of microbial species was identified and selected as an inoculum. The soil-compost mixture, based on genomic analysis using the 16S rRNA gene (prokaryotes) and the 18S gene (eukaryotes), contained a diverse group of bacteria, fungi, and archaea. Bacterial species contained in the soil-compost mixture included members from Acidobacteria, Actinobacteria, Bacteroidetes, Chlamydiae, Chloroflexi, Cyanobacteria, Deinococcus-Thermus, Firmicutes, Gemmatimonadetes, Hydrogenedentes, Nitrospirae, Parcubacteria, Planctomycetes, Proteobacteria, Saccharibacteria, Spirochaetes, Tenericutes, Thaumarchaeota, Verrucomicrobia, and yet unclassified taxa (FIG. 2).

Four compartments (surfaces with 1 mm holes) were each filled with ~10-15 kg of the soil-compost mixture at 15-20% moisture (by weight). The four compartments were suspended in a 145-gallon HD polyethylene cylindrical open-top water tank (42 inches in diameter, and 54 inches deep) containing about 110 gallons of chlorine-free tap water such that the tops of the compartments were slightly above the surface of the water. The water was allowed to saturate the soil-compost mixtures and yield a cell bath mixture. The four compartments filled with the soil-compost mixture displaced 10 gallons of water. Thus, the ratio of the soil-compost solids-to-water volumetric ratio was about 1:11.

Sugar addition: After starting to aerate the contents of the open-top water tank by bubbling, 1 gallon of syrup was poured into the tank. The syrup was a mixture of molasses and corn syrup (which may have contained high fructose corn syrup). This translated to a sucrose sugar (1:1 glucose:fructose) equivalent of about 4,040 g per 110 gallons of water (and a negligible 5 g NaCl per 110 gallons of water), making the volumetric sugar concentration 9%, or 28 mM.

### Aeration Phase (Days 1-3) and Resting Phase (Days 4-10)

Process conditions were measured during the Aeration (alternatively, “aerobic phase”) and Resting phases (alternatively, “anaerobic phase”) using A YSI® multi-sensor sonde connected to a YSI® 650 MDS datalogger (Yellow Springs, OH) was used to measure cell bath mixture temperature (°C), pH, electrical conductivity (µS m⁻¹), dissolved oxygen concentration (mg 1⁻¹), and oxygen saturation (%). Sensors on the sonde were positioned 30 cm below the surface of the cell bath mixture, and values were recorded every 15 minutes.

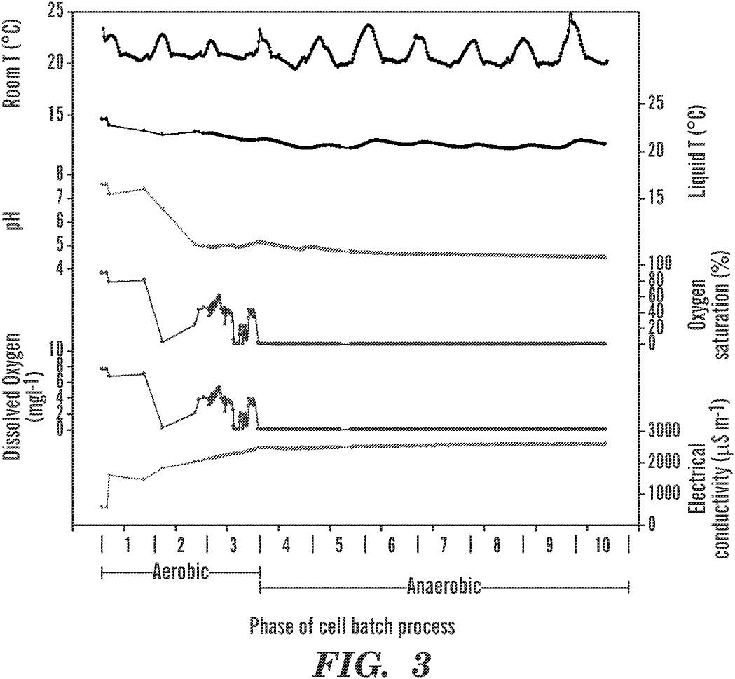

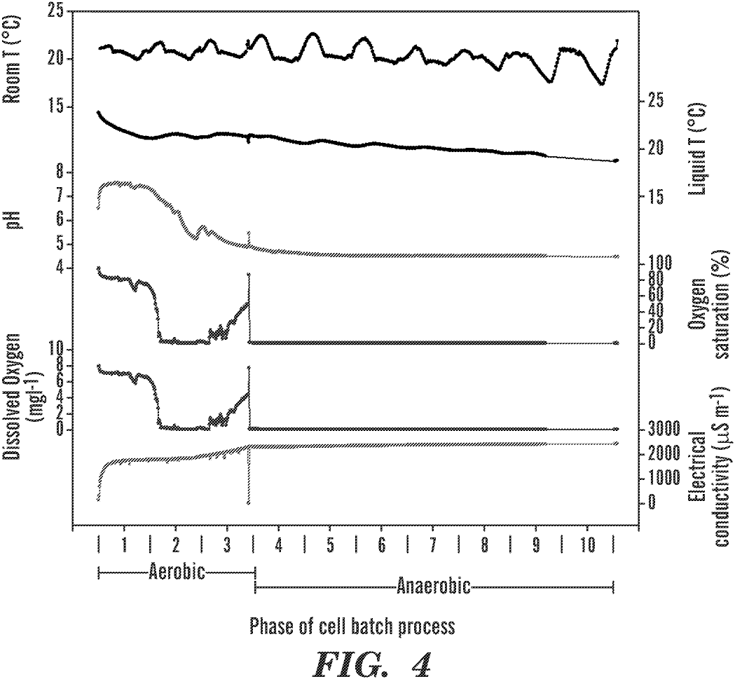

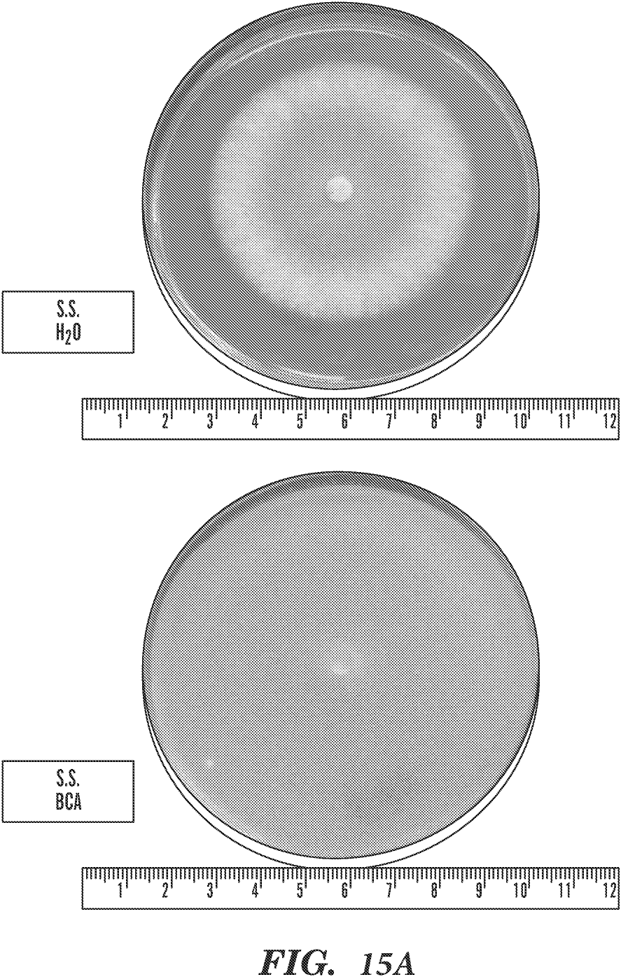

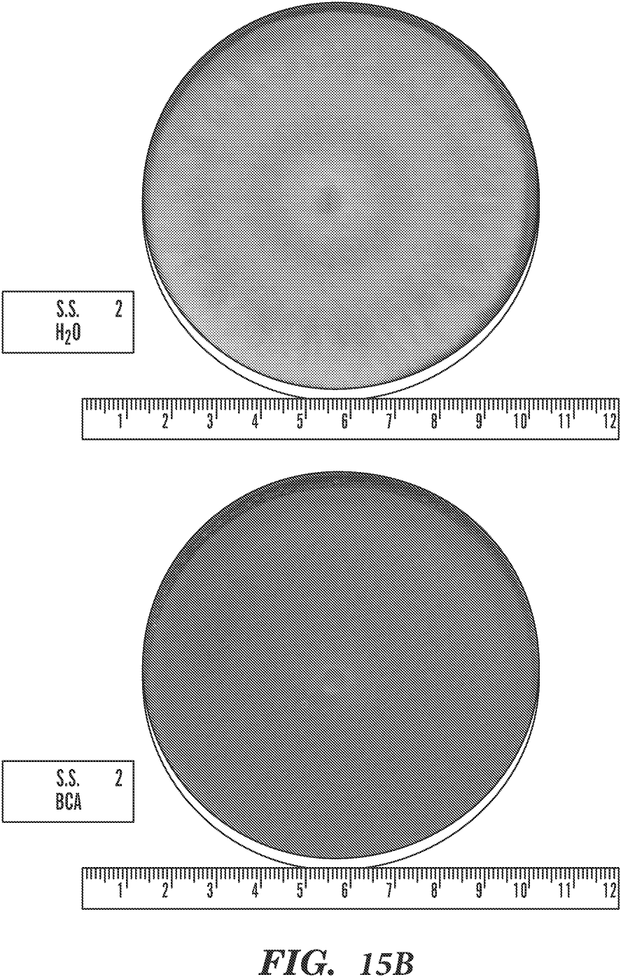

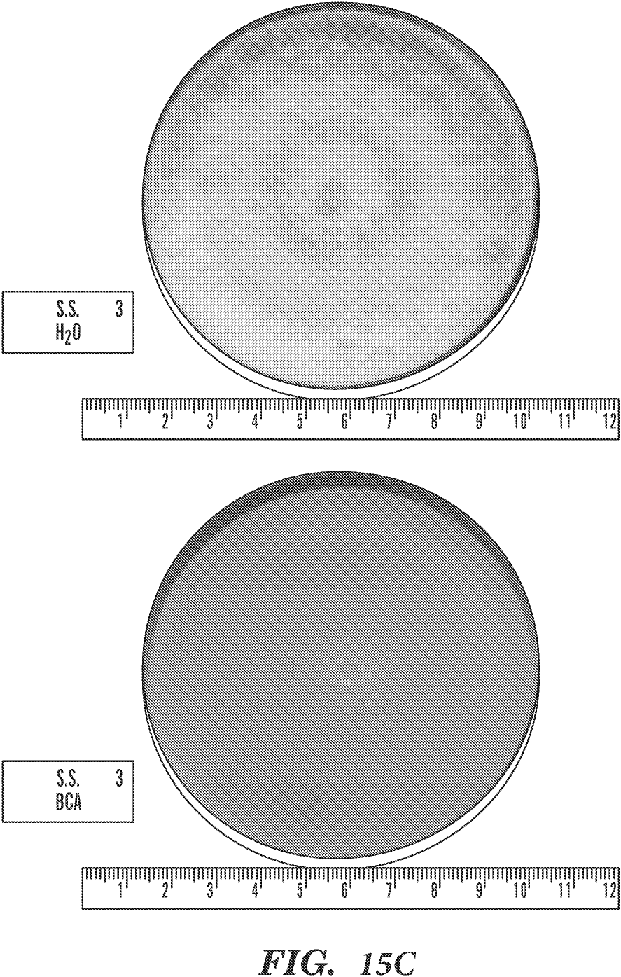

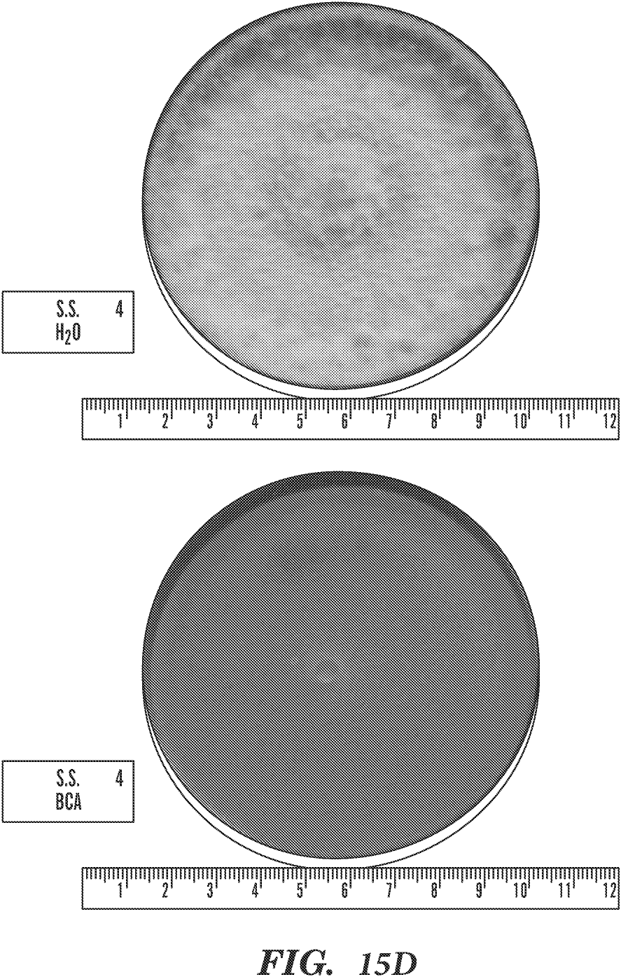

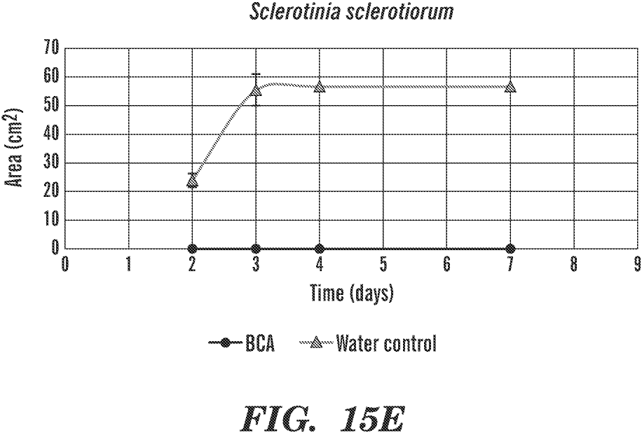

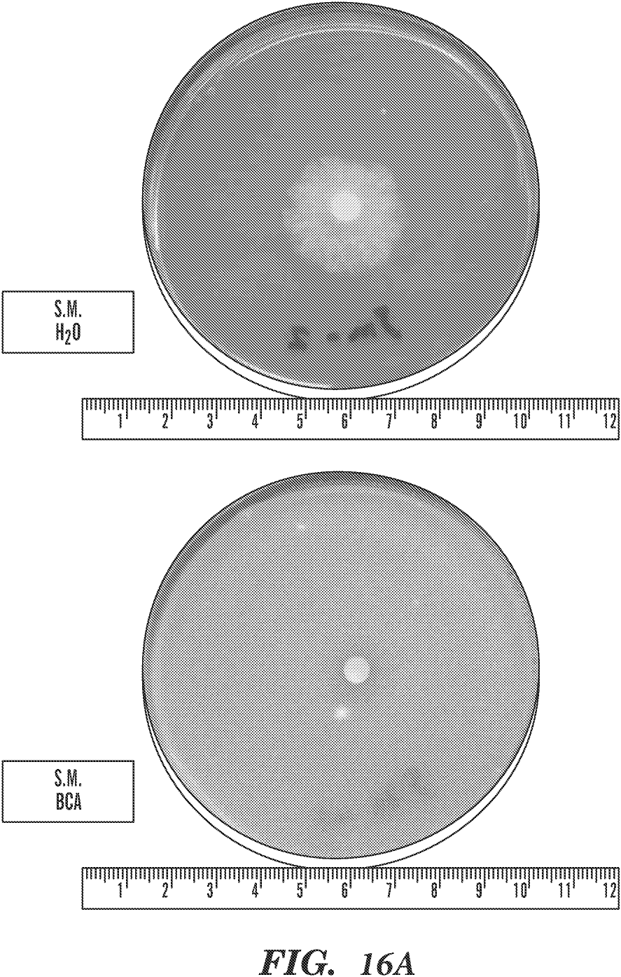

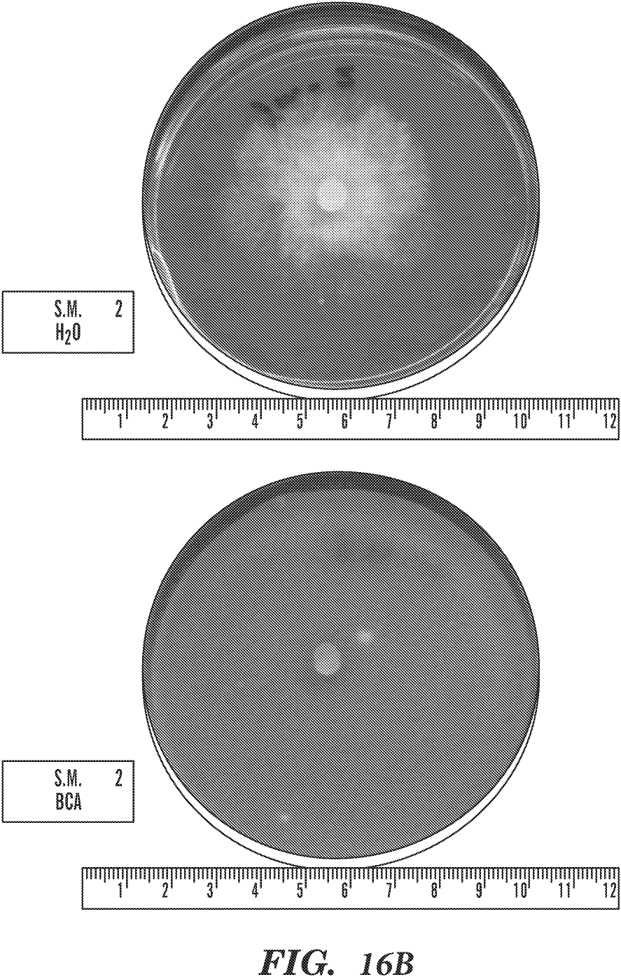

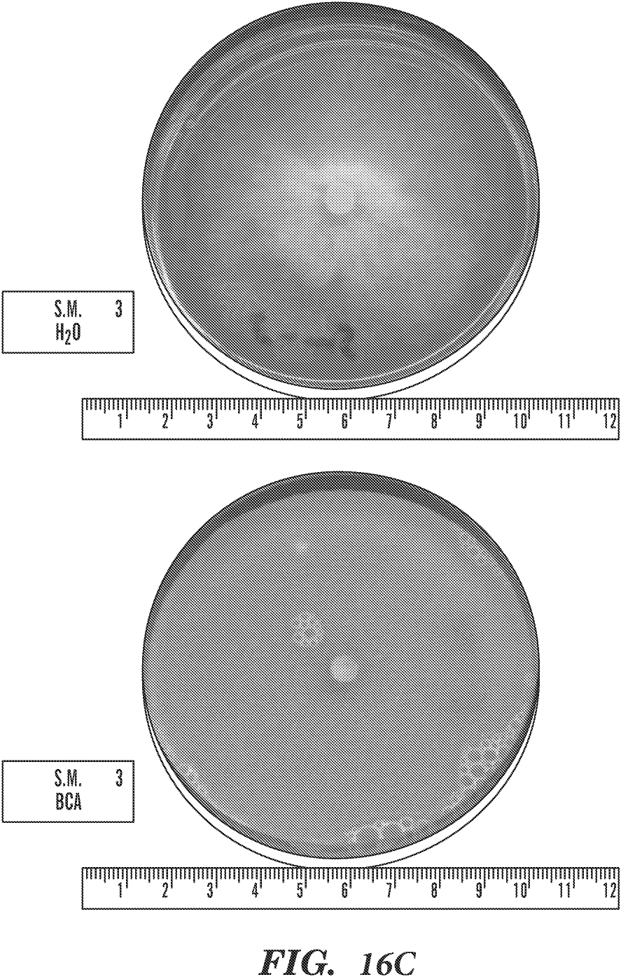

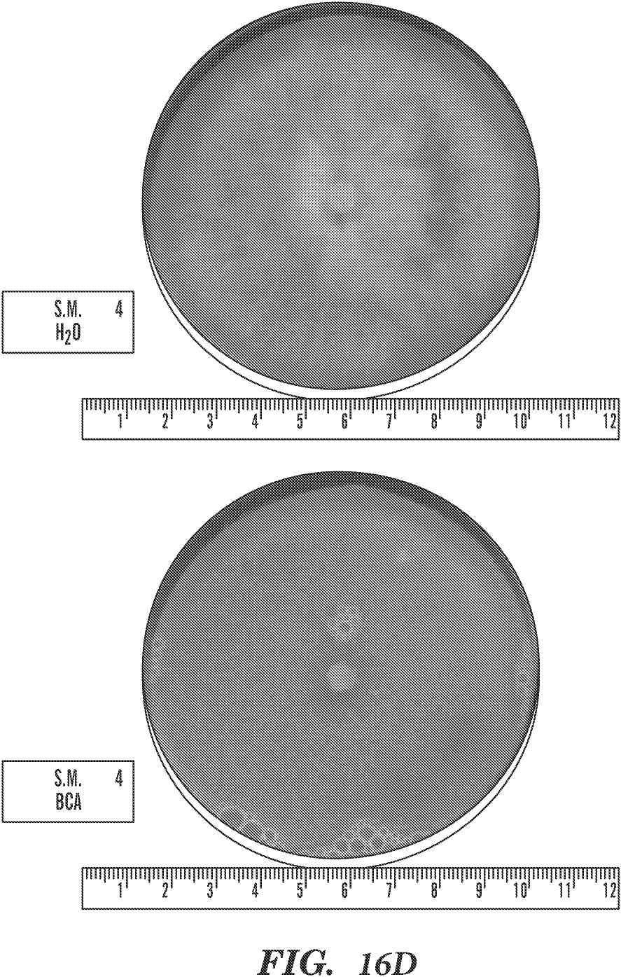

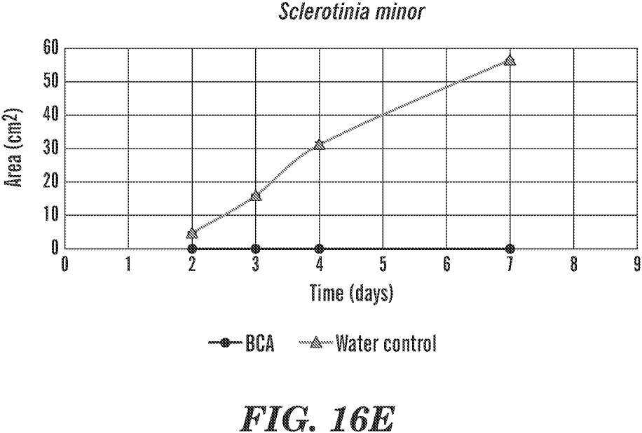

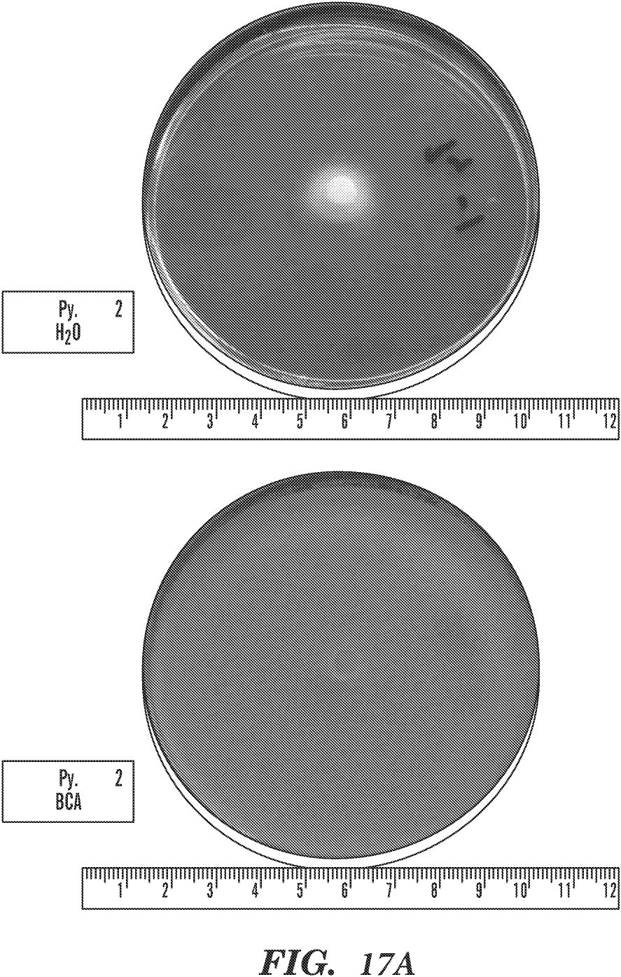

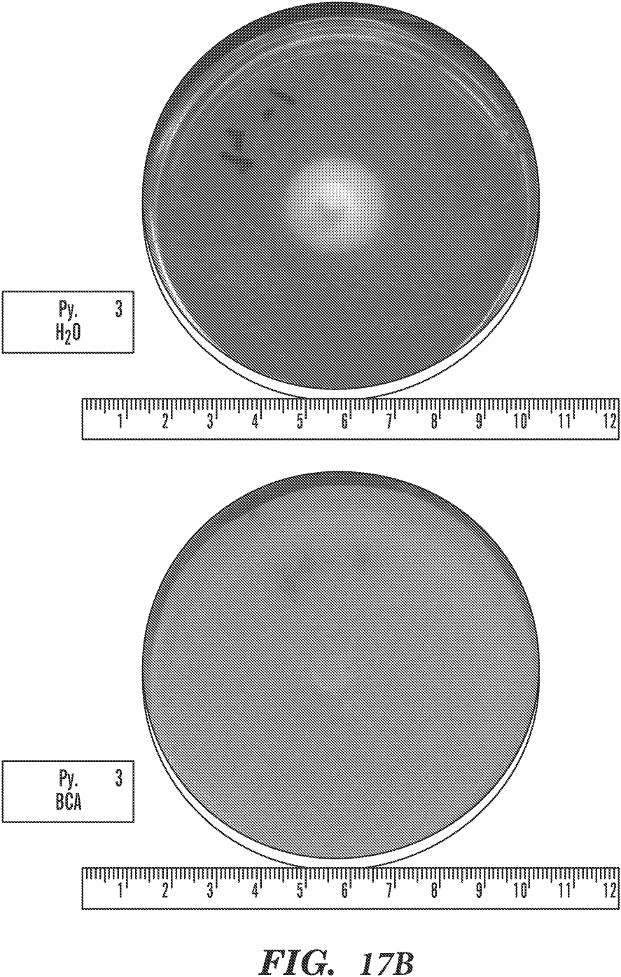

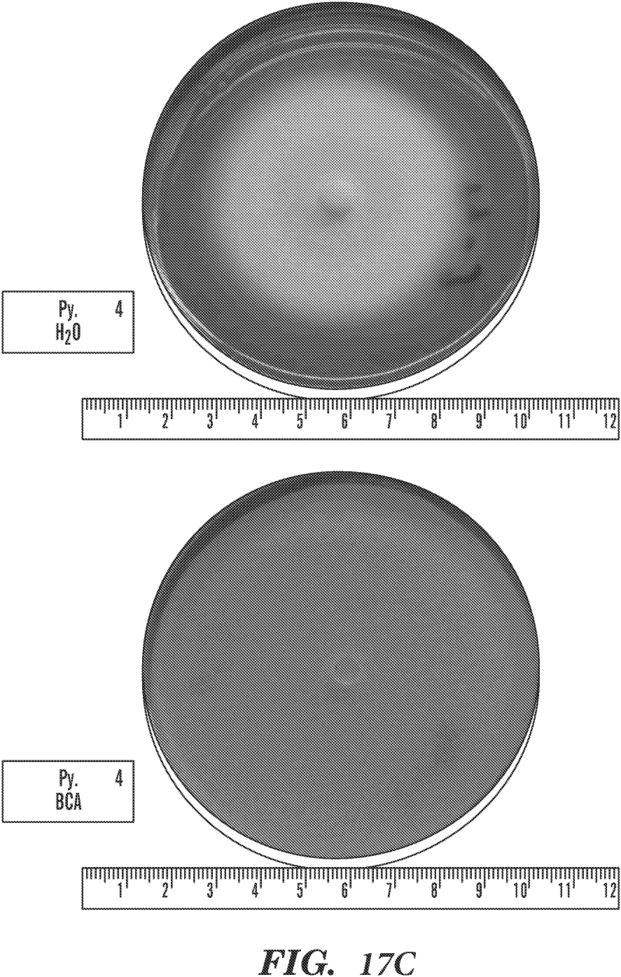

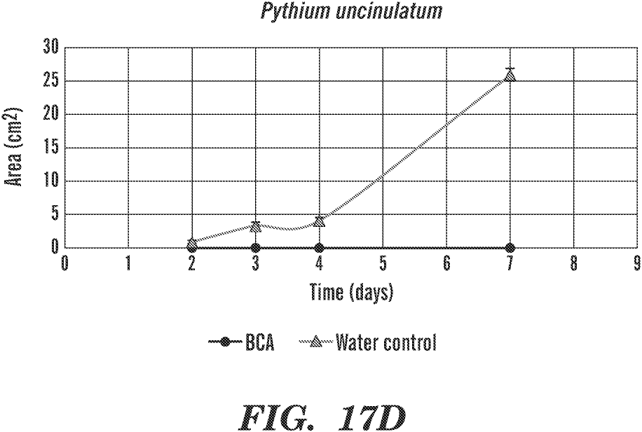

The temperature of the cell bath mixture was maintained at around 21° C. At this temperature 21° C., the biocontrol agent produced showed consistent efficacy against fungal pathogens (see, e.g., Examples 2-8 and FIGS. 15A-18E, and 28A-28H). Diel swings in ambient temperature were observed and were less than about 3.5° C. (see FIGS. 3 and 4). However, cell bath mixture temperatures remained relatively constant with only very slight diel fluctuations. The temperature of the cell bath mixture ranged from 19° C. to 22.5° C. during preparation of the first batch of biocontrol agent (FIG. 3) and from 18.7 to 23.8° C. during preparation of the second batch of biocontrol agent (FIG. 4). The temperature of the cell bath mixture during preparation of the first batch of the biocontrol agent averaged 20.8° C. and the average was 20.7° C. during preparation of the second batch of the biocontrol agent.

During the aeration phase, the temperature of the cell bath mixture during preparation of the first batch of the biocontrol agent averaged 21.5° C. and the average was 21.6° C. during preparation of the second batch of the biocontrol agent.

During the resting phase, the temperature of the cell bath mixture during preparation of the first batch of the biocontrol agent ranged from 20.3° C. to 21.3° C. (mean of 20.7° C.) (FIG. 3). During the resting phase, the temperature of the cell bath mixture during preparation of the second batch of the biocontrol agent ranged from 18.7° C. to 21.4 (mean of 20.2) (FIG. 4).

Ambient air temperatures were measured using a copper-constantan thermocouple positioned near the open-top water tank, and values were recorded every 30 min with a Campbell Scientific Inc. CR1000 datalogger (Logan, UT). Ambient light levels were very low during the day (one north-facing window in the room in which the tank was disposed). There was no ambient light at night.

The bottom of the tank was slightly conical, sloping gently to a 4-inch diameter outlet drain at the bottom fitted with a cam-lock spigot into which air was pumped through a 1-inch diameter reinforced air hose to aerate the cell bath mixture. The aeration created a gentle bubbling of the cell bath mixture. The flow rate of the air was from about 8 cubic feet per minute to about 20.6 cubic feet per minute. The air was pumped into the tank for aeration using a HG-250-C one-speed 110 V, 250 Watt (at 1.75 psi) regenerative rotary air pump. Flow of air into the bottom of the tank was adjusted using an air bleed valve inserted in the path of air flow.

Oxygen levels measured during the 3-day aeration phase ranged from 0.05 mgl⁻¹ during Day 2 to 7.93 mg 1⁻¹ at the start of Day 1 during preparation of the first batch of biocontrol agent. These values corresponded to oxygen saturation values of 0.6% and 89.9%. Mean values measured over the entire 3-day aeration phase during the preparation of the first batch of the biocontrol agent were: 3.05 mg 1⁻¹ and a saturation of 34.9%. During preparation of the second batch of the biocontrol agent, oxygen levels ranged from 0.07 mg 1⁻¹ to 7.97 mg 1⁻¹, corresponding to saturations of 0.7% and 94.3%, respectively. Mean oxygen values were 3.27 mg 1⁻¹ (34.5% saturation), calculated during the 3-day aeration phase during preparation of the second batch of the biocontrol agent.

The time course of oxygen levels typically demonstrated an initial gradual decline during the first day of the aeration phase, followed by precipitous declines near the start of Day 2 when complete anaerobic conditions were reached (FIGS. 3 and 4). Despite aeration, anaerobic oxygen levels remained at anaerobic levels for a full day (Day 2) before recovering partially to about half the level measured at the beginning of the bubbling phase. Oxygen levels during Day 3 of the aeration phase varied considerably during preparation of both batches of the biocontrol agent, with near anaerobic conditions observed for periods of up to 30 min (batch two) to 75 min (batch one). Consequently, during the aeration phase, oxygen levels remained at anaerobic levels.

Oxygen levels measured during the resting phase ranged from 0.00 mg 1⁻¹ to 0.12 mg 1⁻¹ (mean of 0.05 mg 1⁻¹) during preparation of the first batch of the biocontrol agent, which corresponded to oxygen saturation values of from 0.0% to 1.0% (mean of 0.5%). During preparation of the second batch of the biocontrol agent, oxygen values ranged from 0.06 mg 1⁻¹ to 0.15 mg 1⁻¹ (mean: 0.08 mg 1⁻¹), which corresponded to oxygen saturation values of from 0.6% to 1.7% (mean of 0.87%).

The pH of the cell bath mixture typically started at above neutral (7.59 in batch 1; 7.57 in batch 2), remained at this level during Day 1 of aeration, then dropped linearly over the course of Day 2 of the aeration phase to approach the lowest levels observed during preparation of the biocontrol agent (4.93 during preparation of the first batch of biocontrol agent; 4.89 during preparation of the second batch of biocontrol agent) (Days 4-10 of biocontrol agent preparation) by the end of Day 3.

In the resting phase, the pH of the cell bath mixture during preparation of both batches of the biocontrol agent declined slightly from already low levels attained by the end of the aerobic phase (from 5.16 to 4.49, with a mean of 4.68, during preparation of the first batch of the biocontrol agent; and from 5.46 to 4.46, with a mean of 4.54, during preparation of the second batch of the biocontrol agent).

Electrical conductivity (EC) typically increased within the first hours of the aeration phase (from 583 to 1205 µS m⁻¹ during preparation of the first batch of the biocontrol agent; and 23 to 1140 µS m⁻¹ during preparation of the second batch of the biocontrol agent) reaching half maximum values within 3 hours. EC continued to climb to near peak levels by the end of Day 3 of the aeration phase (2420 µS m⁻¹ during preparation of the first batch of the biocontrol agent, 2285 µS m⁻¹ during preparation of the second batch of the biocontrol agent). Mean EC values during the aeration phase during preparation of the first and second batches of the biocontrol agent were 2187 µS m⁻¹ and 1829 µS m⁻¹, respectively

In the resting phase, electrical conductivity (EC) rose slightly from 2432 to 2600 µS m⁻¹ (mean 2532 µS m⁻¹) during preparation of the first batch of the biocontrol agent and from 2302 to 2442 µS m⁻¹ (mean 2370 µS m⁻¹) during preparation of the second batch of the biocontrol agent.

The chemical makeup of the liquid component of the cell bath mixture was measured. The chemicals detected are listed in Tables 1A-1C and 2A-2C. In Tables 1A-1C, lactate, acetate, and propionate levels increased over the 10 days of preparation of the first batch of the biocontrol agent. Phosphate, strontium, propionate, and formate levels increased by approximately 10-fold over the 10 days. Calcium levels increased by 9-fold over the 10 days. Magnesium levels increased by 4-fold over the 10 days. Potassium levels increased by 3-fold over the 10 days. Barium levels became detectable over the 10 days. Fluoride levels had an increase between days 2-6 and returned back to near baseline levels by day 10. Nitrate and succinate levels decreased significantly during the first day. Lithium, sodium, ammonium, nitrite, bromide, and methanesulfonate levels did not significantly change over the 10 days. Similar trends were also observed during preparation of the second batch of the biocontrol agent (Tables 2A-2C).

TABLE 1A

Chemical composition of a first bioreaction

| Sample | Day | Lithium (ppm) | Sodium (ppm) | Ammonium (ppm) | Magnesium (ppm) | Potassium (ppm) | Calcium (ppm) | Strontium (ppm) |
|---|---|---|---|---|---|---|---|---|
| AA | 0 | 0.00277 | 27.03013 | 0.03727 | 3.49603 | 74.71723 | 9.19032 | 0.03085 |
| A | 1 | 0.00535 | 40.90332 | 0.00000 | 5.54801 | 175.10429 | 11.27709 | 0.07817 |
| B | 2 | 0.00636 | 43.69901 | 0.03395 | 14.03644 | 196.87653 | 44.25460 | 0.25456 |
| C | 3 | 0.00663 | 43.27000 | 0.02891 | 20.00053 | 195.73177 | 116.08756 | 0.04487 |
| D | 4 | 0.00678 | 43.06628 | 0.01685 | 20.94652 | 195.55988 | 89.08009 | 0.45039 |
| E | 5 | 0.00505 | 32.75171 | 0.01420 | 15.84966 | 147.21620 | 66.24046 | 0.38042 |
| F | 6 | 0.00495 | 30.56380 | 0.01906 | 15.77940 | 144.72156 | 63.74936 | 0.34723 |
| G | 7 | 0.00622 | 43.64402 | 0.01705 | 20.68261 | 195.24046 | 82.67170 | 0.41003 |
| H | 8 | 0.00433 | 29.59367 | 0.00947 | 14.01705 | 127.17750 | 55.07001 | 0.29490 |
| I | 9 | 0.00858 | 54.72858 | 0.01949 | 26.87339 | 246.63261 | 105.81386 | 0.56741 |
| J | 10 | 0.00637 | 39.76703 | 0.01267 | 19.90709 | 191.35739 | 80.78576 | 0.47546 |

TABLE 1B

Chemical composition of a first bioreaction

| Sample | Day | Barium (ppm) | Fluoride (ppm) | Chlorine (ppm) | Nitrite (ppm) | Bromide (ppm) | Nitrate (ppm) | Sulfate (ppm) | Phosphate (ppm) |
|---|---|---|---|---|---|---|---|---|---|
| AA | 0 | 0.00000 | 0.09222 | 50.18627 | 0.00000 | 0.00000 | 39.52712 | 51.57106 | 2.18040 |
| A | 1 | 0.00000 | 19.76057 | 95.54106 | 0.00000 | 0.00000 | 0.10458 | 118.87971 | 8.32269 |
| B | 2 | 0.00000 | 43.08568 | 97.38150 | 0.00000 | 0.00880 | 0.39618 | 124.65916 | 11.24910 |
| C | 3 | 0.05572 | 48.27472 | 89.63470 | 0.00000 | 0.00791 | 1.05138 | 113.45810 | 26.89377 |
| D | 4 | 0.03583 | 36.83628 | 89.12242 | 0.00000 | 0.00863 | 2.29707 | 109.52849 | 27.80258 |
| E | 5 | 0.04193 | 17.26861 | 64.75592 | 0.00000 | 0.00404 | 3.30580 | 90.70129 | 20.57804 |
| F | 6 | 0.04402 | 8.10445 | 64.90382 | 0.00000 | 0.00365 | 5.66335 | 83.92701 | 19.40453 |
| G | 7 | 0.03789 | 3.55196 | 89.09813 | 0.00000 | 0.00110 | 10.98917 | 108.25885 | 25.64228 |
| H | 8 | 0.03719 | 0.00000 | 56.60369 | 0.00000 | 0.00000 | 8.56794 | 73.58062 | 16.63491 |
| I | 9 | 0.06615 | 0.00000 | 113.20339 | 0.00000 | 0.00016 | 18.13094 | 131.36665 | 32.07876 |
| J | 10 | 0.05013 | 0.00000 | 84.27313 | 0.00000 | 0.00000 | 13.04758 | 112.63309 | 23.78020 |

TABLE 1C

Chemical composition of a first bioreaction

| Sample | Day | Lactate (ppm) | Acetate (ppm) | Propionate (ppm) | Formate (ppm) | Methanesulfonate (ppm) | Succinate (ppm) | Maleate (ppm) | Oxalate (ppm) |
|---|---|---|---|---|---|---|---|---|---|
| AA | 0 | 0.17281 | 0.32233 | 0.00584 | 0.27650 | 0.00000 | 0.00000 | 0.00000 | 0.11714 |
| A | 1 | 24.45638 | 20.77058 | 0.11278 | 0.19480 | 0.00000 | 1.61231 | 0.05173 | 0.01450 |
| B | 2 | 64.79023 | 153.35220 | 0.36570 | 1.03447 | 0.00000 | 4.46246 | 0.62219 | 0.51097 |
| C | 3 | 131.43981 | 195.61953 | 0.32379 | 0.96091 | 0.00000 | 5.33085 | 0.60801 | 1.13264 |
| D | 4 | 230.15110 | 235.84194 | 0.30872 | 0.90506 | 0.00000 | 6.12735 | 0.27427 | 0.87704 |
| E | 5 | 213.93778 | 246.09479 | 0.28122 | 2.21246 | 0.00000 | 4.21369 | 0.19793 | 0.55028 |
| F | 6 | 220.81754 | 291.33604 | 0.24513 | 2.46445 | 0.00000 | 2.82426 | 0.18283 | 0.55344 |
| G | 7 | 312.89800 | 383.66656 | 0.22759 | 3.57917 | 0.00000 | 2.39566 | 0.26700 | 0.60146 |
| H | 8 | 195.66152 | 289.34696 | 0.23791 | 2.27215 | 0.00000 | 0.87709 | 0.18325 | 0.39786 |
| I | 9 | 355.14177 | 498.31815 | 0.40354 | 4.25492 | 0.00000 | 0.00000 | 0.32698 | 0.72584 |
| J | 10 | 288.23901 | 389.29340 | 0.36230 | 3.02160 | 0.00000 | 0.00000 | 0.16579 | 0.51481 |

TABLE 2A

Chemical composition of a second bioreaction

| Sample | Day | Lithium (ppm) | Sodium (ppm) | Ammonium (ppm) | Magnesium (ppm) | Potassium (ppm) | Calcium (ppm) | Strontium (ppm) |
|---|---|---|---|---|---|---|---|---|
| KK | 0 | 0.00173 | 16.00456 | 0.02102 | 1.90989 | 40.71428 | 5.38912 | 0.01760 |
| K | 1 | 0.00325 | 26.29762 | 0.00000 | 3.16741 | 106.22490 | 7.32182 | 0.05222 |
| L | 2 | 0.00656 | 44.09099 | 0.00128 | 11.91481 | 194.85008 | 32.04046 | 0.20126 |
| M | 3 | 0.00574 | 37.99111 | 0.02127 | 16.61775 | 165.17392 | 55.50536 | 0.33927 |
| N | 4 | 0.00618 | 37.60005 | 0.01834 | 17.64017 | 178.45201 | 60.62689 | 0.38326 |
| O | 5 | 0.00827 | 52.38524 | 0.01904 | 23.90367 | 236.93255 | 88.88018 | 0.48807 |
| P | 6 | 0.00441 | 29.93229 | 0.01278 | 13.28571 | 130.60288 | 50.02608 | 0.28227 |
| Q | 7 | 0.00858 | 55.48060 | 0.01774 | 24.81371 | 253.18255 | 96.72753 | 0.54640 |
| R | 8 | 0.00543 | 33.71173 | 0.00948 | 15.98501 | 154.93057 | 59.47762 | 0.30979 |
| S | 9 | 0.00435 | 29.10719 | 0.00951 | 13.08500 | 130.82400 | 51.16529 | 0.35543 |
| T | 10 | 0.00623 | 39.82769 | 0.01568 | 18.80516 | 184.77082 | 69.85989 | 0.46489 |

TABLE 2B

Chemical composition of a second bioreaction

| Sample | Day | Barium (ppm) | Fluoride (ppm) | Chlorine (ppm) | Nitrite (ppm) | Bromide (ppm) | Nitrate (ppm) | Sulfate (ppm) | Phosphate (ppm) |
|---|---|---|---|---|---|---|---|---|---|
| KK | 0 | 0.00000 | 0.08100 | 32.78173 | 0.00000 | 0.00000 | 22.30007 | 25.66068 | 1.24489 |
| K | 1 | 0.00000 | 7.28888 | 58.40094 | 0.00000 | 0.00000 | 0.11271 | 69.62289 | 2.32021 |
| L | 2 | 0.00000 | 7.50462 | 98.80010 | 0.00000 | 0.00178 | 0.72231 | 122.24022 | 9.91287 |
| M | 3 | 0.02409 | 46.82740 | 78.93784 | 0.00000 | 0.00224 | 0.92743 | 102.23648 | 23.63659 |
| N | 4 | 0.03164 | 36.47063 | 83.70713 | 0.00000 | 0.00323 | 1.69084 | 105.52483 | 24.67161 |
| O | 5 | 0.04202 | 31.09503 | 108.64544 | 0.00000 | 0.00231 | 3.89101 | 135.82983 | 32.98675 |
| P | 6 | 0.01466 | 9.53750 | 59.55571 | 0.00000 | 0.00040 | 3.78845 | 82.89827 | 17.10329 |
| Q | 7 | 0.05274 | 17.16814 | 116.58826 | 0.00000 | 0.00077 | 8.35921 | 148.38870 | 30.94642 |
| R | 8 | 0.05498 | 7.62308 | 71.79118 | 0.00000 | 0.00027 | 6.90759 | 91.12386 | 21.14842 |
| S | 9 | 0.03100 | 3.27291 | 56.65789 | 0.00000 | 0.00000 | 7.36412 | 94.66430 | 17.31605 |
| T | 10 | 0.06629 | 1.22831 | 83.37042 | 0.00000 | 0.00000 | 11.65849 | 120.83483 | 24.62127 |

TABLE 2C

Chemical composition of a second bioreaction

| Sample | Day | Lactate (ppm) | Acetate (ppm) | Propionate (ppm) | Formate (ppm) | Methanesulfonate (ppm) | Succinate (ppm) | Maleate (ppm) | Oxalate (ppm) |
|---|---|---|---|---|---|---|---|---|---|
| KK | 0 | 0.12138 | 0.48800 | 0.00610 | 0.22190 | 0.00000 | 0.00000 | 0.00000 | 0.06559 |
| K | 1 | 0.00000 | 28.24726 | 0.13440 | 0.09368 | 0.00000 | 0.62281 | 0.00081 | 0.06874 |
| L | 2 | 188.48532 | 103.98015 | 0.23046 | 0.21419 | 0.00000 | 16.35903 | 0.25333 | 0.13412 |
| M | 3 | 143.76830 | 126.79276 | 0.38504 | 0.36163 | 0.00000 | 19.64002 | 0.95752 | 0.38571 |
| N | 4 | 226.14740 | 209.93616 | 0.30584 | 0.37451 | 0.00000 | 21.97613 | 0.09318 | 0.57855 |
| O | 5 | 316.33933 | 340.24825 | 0.31357 | 4.20446 | 0.00000 | 27.92473 | 0.15157 | 0.73104 |
| P | 6 | 168.02961 | 255.10678 | 0.29732 | 1.20708 | 0.00000 | 9.42637 | 0.08670 | 0.49544 |
| Q | 7 | 321.88736 | 450.89040 | 0.29138 | 1.56875 | 0.00000 | 18.14884 | 0.17424 | 0.64527 |
| R | 8 | 204.17324 | 321.65387 | 0.28555 | 1.21083 | 0.00000 | 9.38085 | 0.10442 | 0.27772 |
| S | 9 | 165.05080 | 270.63594 | 0.27553 | 1.56552 | 0.00000 | 5.66748 | 0.07983 | 0.22279 |
| T | 10 | 230.17257 | 361.42583 | 0.26916 | 1.77582 | 0.00000 | 4.73610 | 0.11710 | 0.32085 |

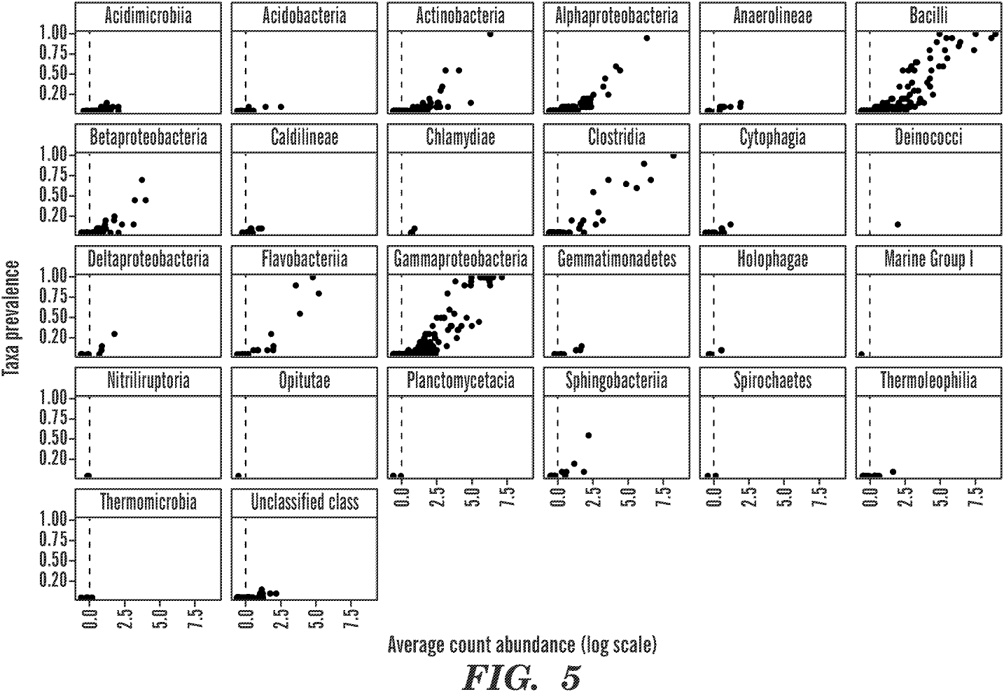

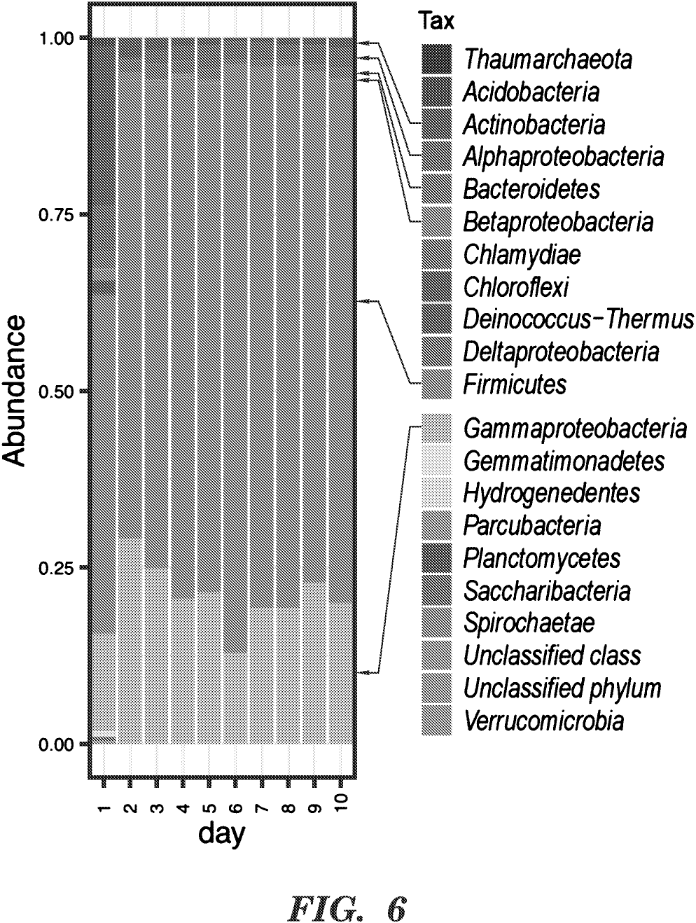

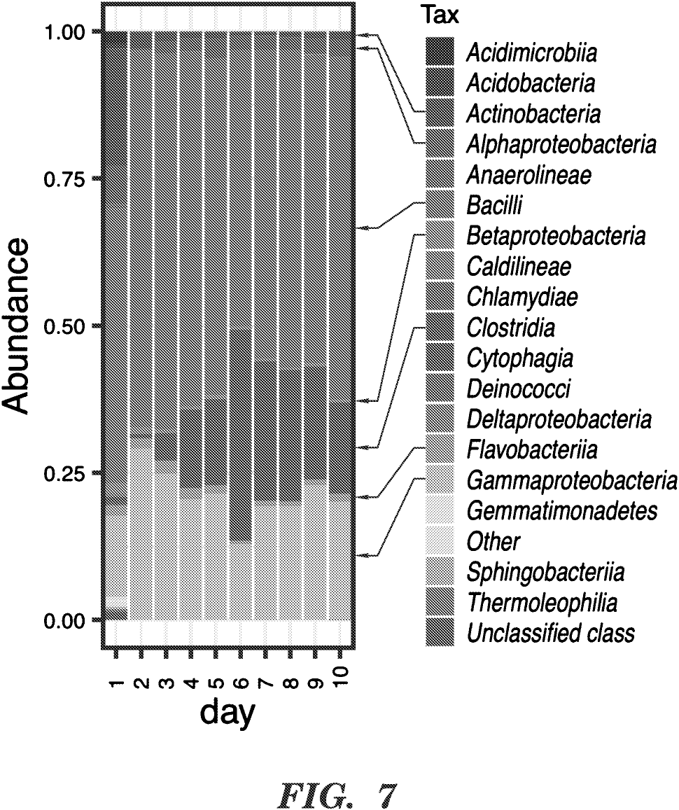

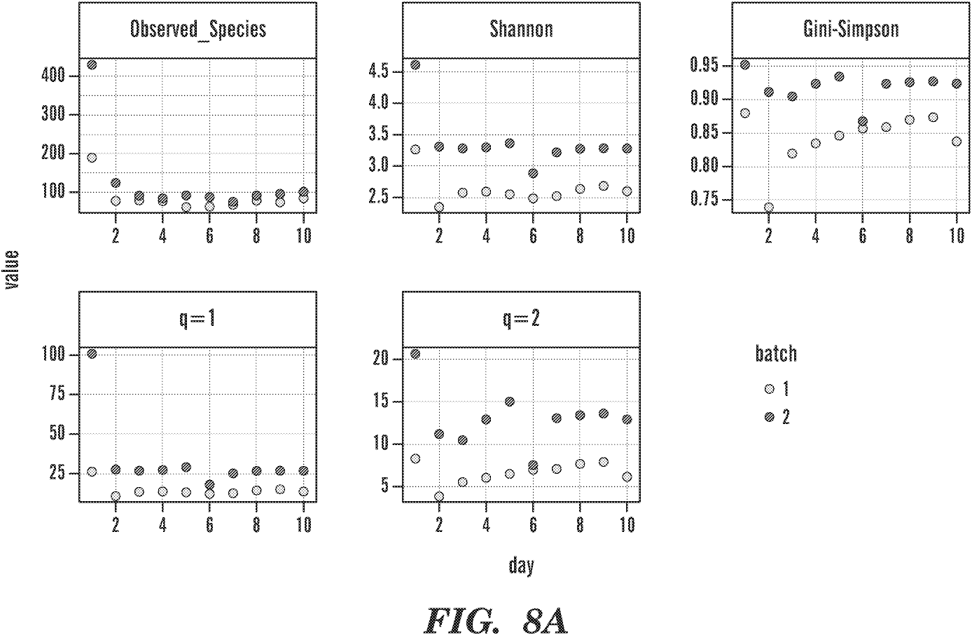

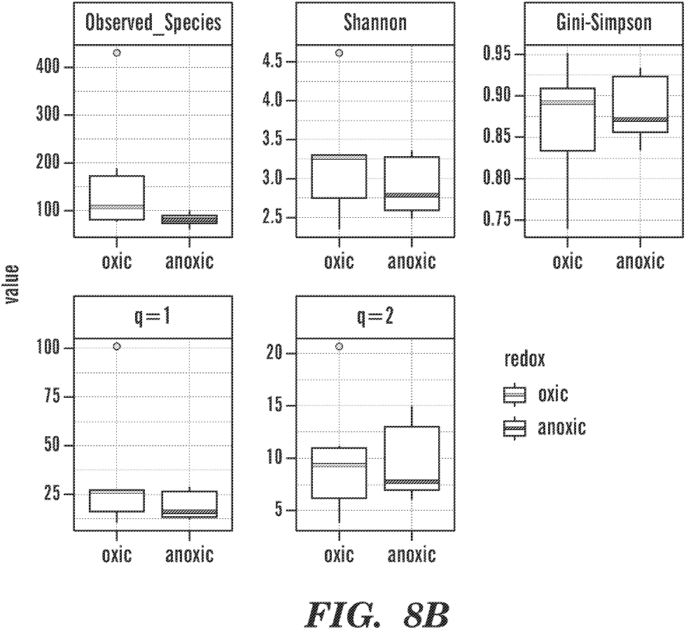

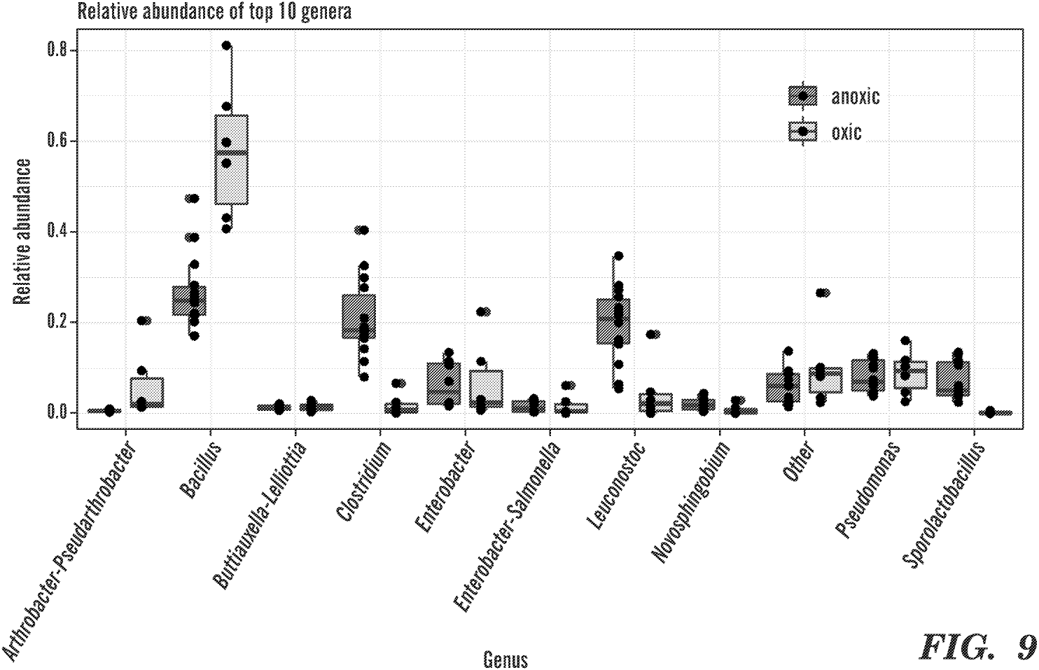

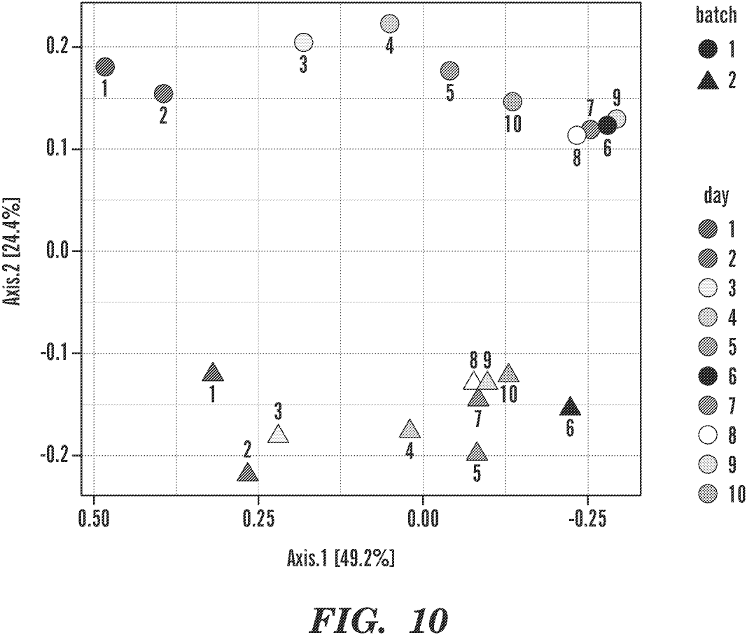

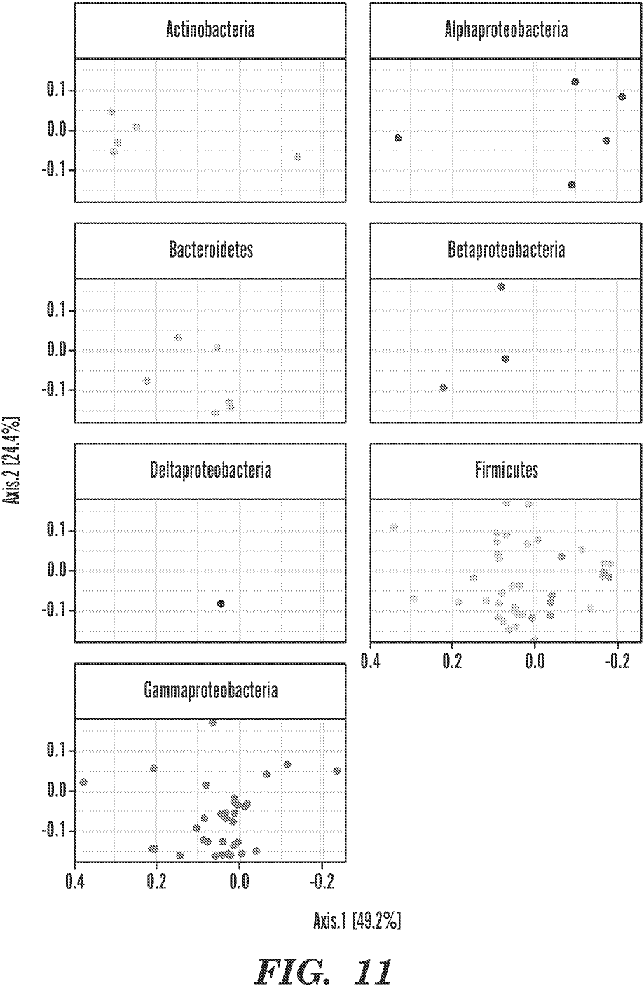

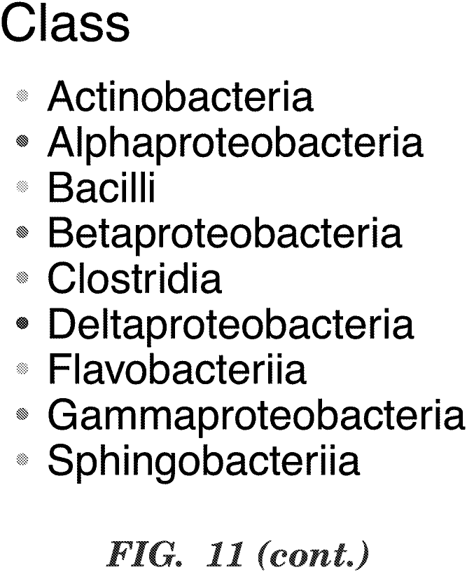

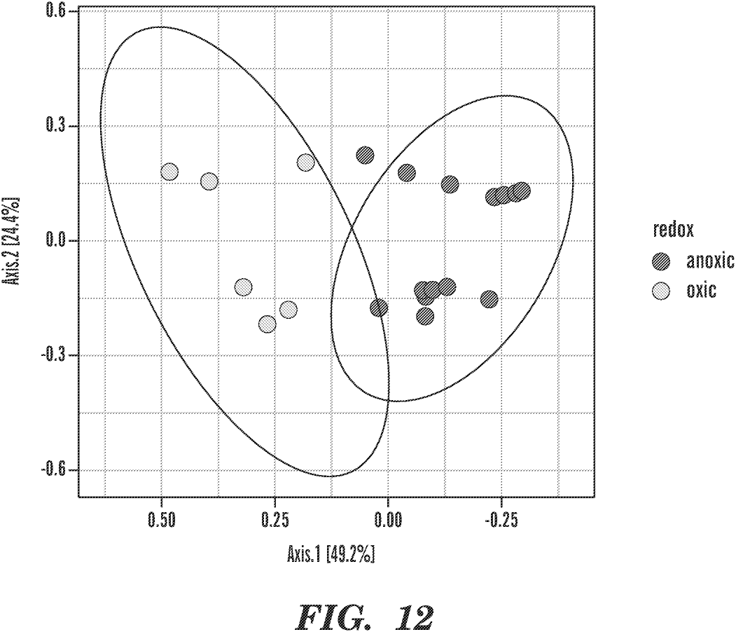

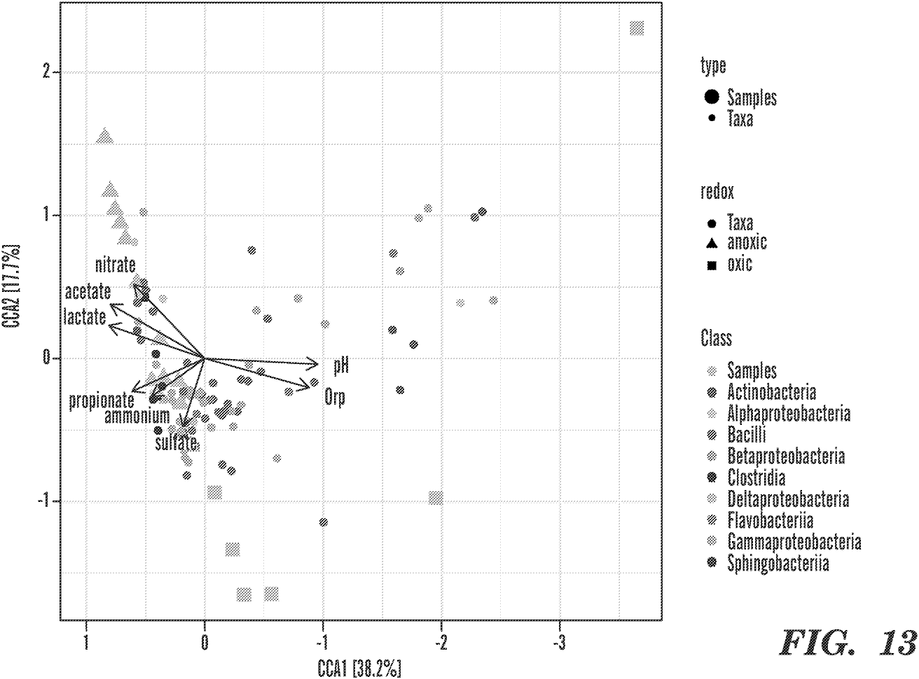

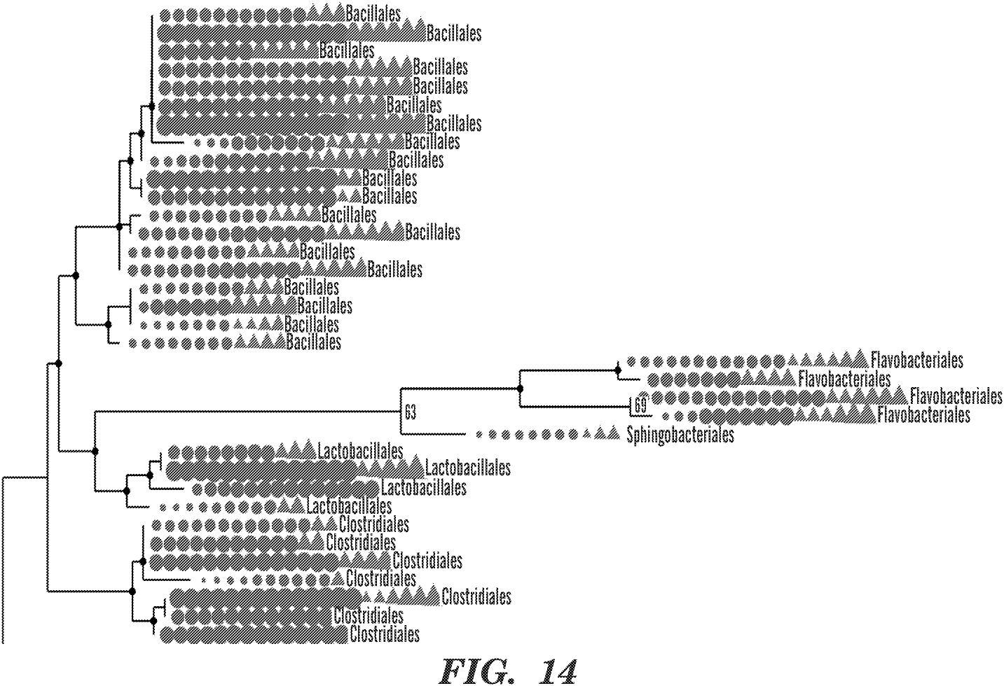

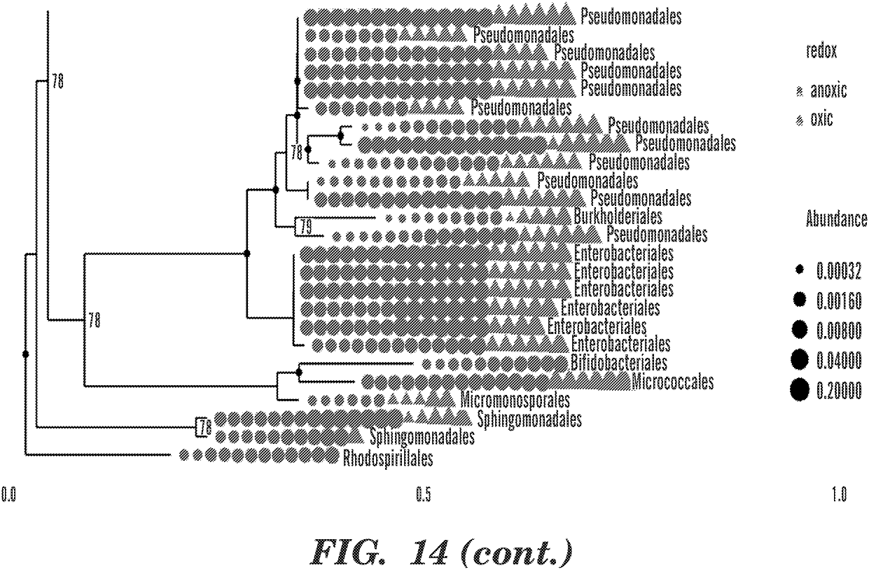

Over the course of preparation of both batches of the biocontrol agent, there were shifts in the relative abundance of bacterial species from the following phyla: Acidobacteria, Actinobacteria, Bacteroidetes, Chlamydiae, Chloroflexi, Cyanobacteria, Deinococcus - Thermus, Firmicutes, Gemmatimonadetes, Hydrogenedentes, Nitrospirae, Parcubacteria, Planctomycetes, Proteobacteria, Saccharibacteria, Spirochaetes, Tenericutes, Thaumarchaeota, Verrucomicrobia, and yet unclassified taxa comprising an additional fraction of the overall bacterial community during preparation of the biocontrol agents (FIGS. 5-14, which represent averages over both batches of the biocontrol agent)

### Filtration

After the resting phase, the cell bath mixture was passed through two cylindrical hollow fiber ultrafiltration membrane modules with a nominal pore size of 0.05 µm (molecular weight limit of 100,000 Daltons). The resulting filtrate, which was the biocontrol agent, which was substantially free of microbial content, the biocontrol agent was conveyed by osmotic pressure into storage tanks of varying capacity.

### Example 2. The Biocontrol Agent of Example 1 Inhibits Growth of Sclerotinia Sclerotiorum, Sclerotinia Minor, and Pythium Uncinulatum

The filtered compositions produced in Example 1 (i.e., the biocontrol agents (BCA)) were evaluated in vitro for their influence on the growth of the fungal pathogens Sclerotinia sclerotiorum, Sclerotinia minor, and Pythium uncinulatum. Various species of Pythium and Sclerotinia fungi are important plant pathogens in agricultural and horticultural industries worldwide. Both fungal groups affect dozens of commercial crops and can cause significant losses of commodity quality, yields, and profit. Pythium species are most often associated with young seedling root rots and plant decline and death. Pythium uncinulatum causes root rot and plant death of lettuce and has become an economically damaging pathogen in California. Of the various Sclerotinia species, Sclerotinia sclerotiorum and Sclerotinia minor are the two economically most important pathogens on crops. Both species have very broad host ranges and cause crown rots of many plants. In addition, Sclerotinia sclerotiorum has an aerial spore stage that results in foliar blights and rots.

Growth of the fungal pathogens was evaluated in triplicate on potato dextrose agar (PDA) containing the biocontrol agent. As a negative control, growth was evaluated in triplicate on PDA containing water in place of the biocontrol agent. To prepare the PDA, 180 ml of streptomycin-PDA was combined with either 180 ml of the biocontrol agent or 180 ml MilliQ water. The PDA was inoculated on the same day that it was prepared. Inoculation of the potato dextrose agar (PDA) involved placing a 5-mm diameter agar plug from PDA containing a mycelial culture of a plant pathogen onto the center of a petri plate containing the prepared PDA. Inoculated plates were incubated at room temperature in a darkened incubator. Images were taken of the petri plates at 2, 3, 4, and 7 days, see FIGS. 15A-15D, 16A-16D, and 17A-17C. Area of fungal growth was calculated from the images using ImageJ software, Table 3 and FIGS. 15E, 16E, and 17D.

TABLE 3

Fungal Growth as measured by colony area.

| Sclerotinia sclerotiorum | Colony area (sq. cm)* | | | |
|---|---|---|---|---|
| Treatment | Davy 2 | Day 3 | Day 4 | Day 7 |
| BCA filtered | 0.2 | 0.2 | 0.2 | 0.2 |
| Water control | 24.0 | 55.6 | 56.7 | 56.7 |
| Sclerotinia minor | Colony area (sq cm)* | | | |
| Treatment | Day 2 | Day 3 | Day 4 | Day 7 |
| BCA filtered | 0.2 | 0.2 | 0.2 | 0.2 |
| Water control | 4.9 | 16.1 | 31.2 | 56.7 |

| Pythium uncinulatum | Colony area (sq cm)* | | | |
|---|---|---|---|---|
| Treatment | Day 2 | Day 3 | Day 4 | Day 7 |
| BCA filtered | 0.2 | 0.2 | 0.2 | 0.2 |
| Water control | 0.9 | 3.3 | 4.1 | 26.0 |

*0.2 sq. cm = area of agar plug and therefore indicates no growth. 56.7 sq. cm = maximum area of the petri dish

Growth of Sclerotinia sclerotiorum on the water control was rapid and reached the maximum area (total area of the petri plate) by day 4. No growth occurred on any day for the biocontrol agent (BCA) plates.

Growth of Sclerotinia minor on the water control was robust, but slower than growth observed with S. sclerotiorum, and reached the maximum area (total area of the petri plate) by day 7. No growth occurred on any day for the biocontrol agent (BCA) plates.

Growth of Pythium uncinulatum, a slow growing species of Pythium, was observed on the water control plates and the colony area measuring 26 sq. cm by day 7. No growth occurred on any day for the BCA plates.

### Example 3. The Biocontrol Agent of Example 1 Inhibits Growth of Colletotrichum Acutatum, Fusarium Oxysporum, Macrophomina Phaseolina, Phytophthora Cactorum, and Verticillium Dahliae

The filtered compositions produced in Example 1 (i.e., the biocontrol agents (BCA)) were evaluated in vitro for their influence on the growth of the fungal plant pathogens Colletotrichum acutatum, Fusarium Oxysporum, Macrophomina phaseolina, Phytophthora cactorum, and Verticillium dahliae.

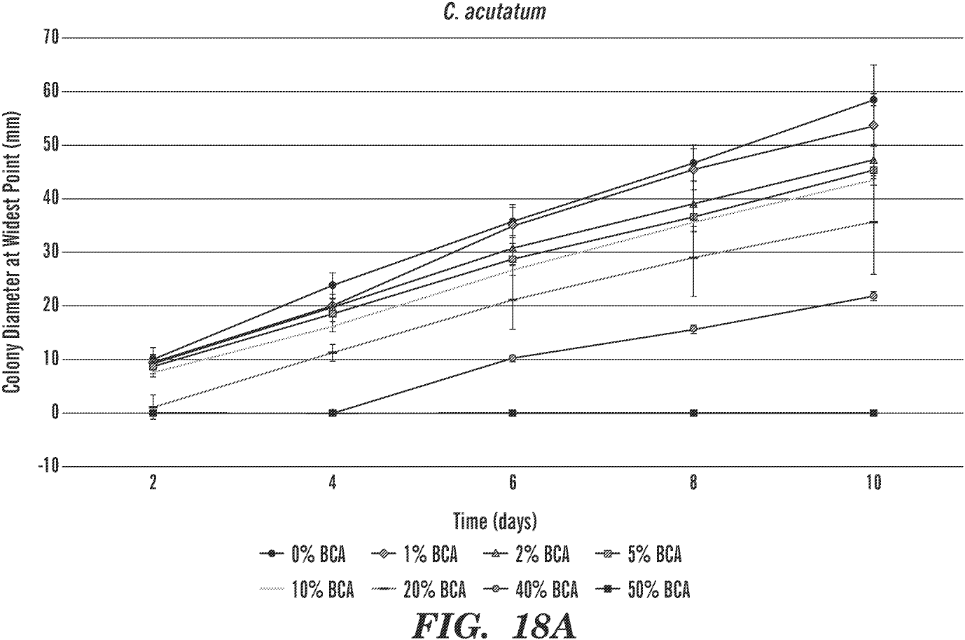

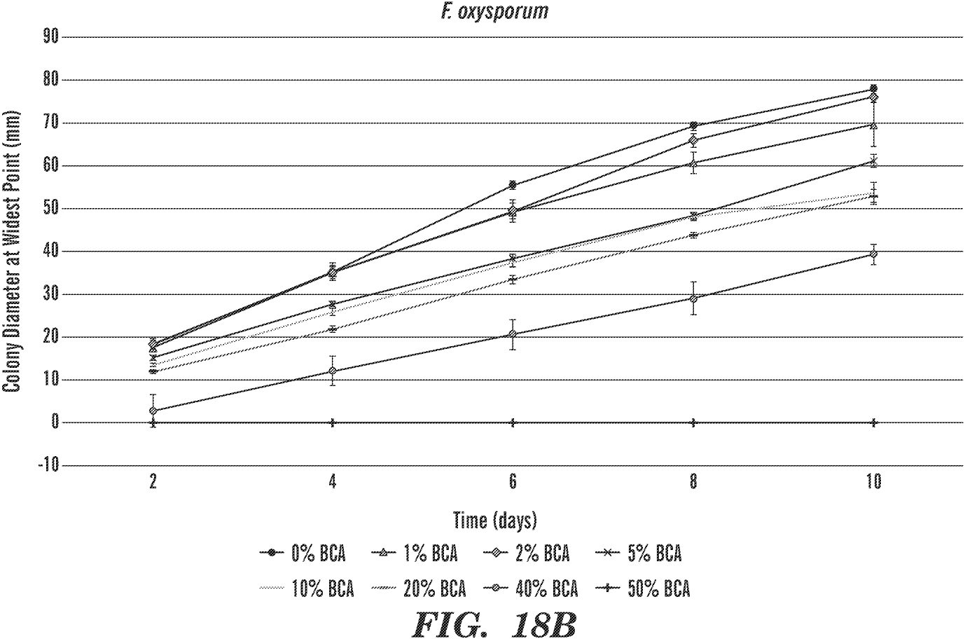

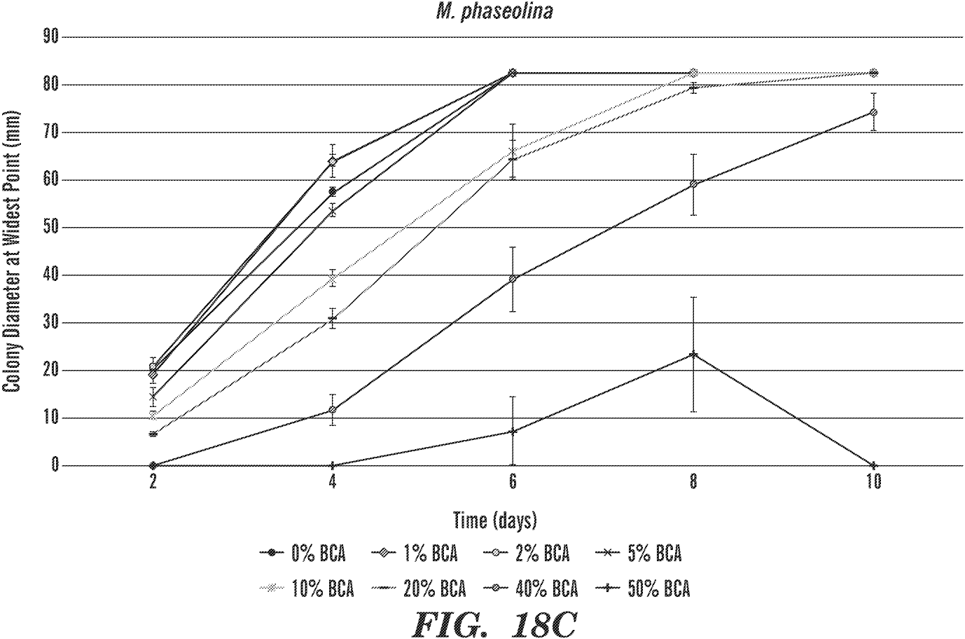

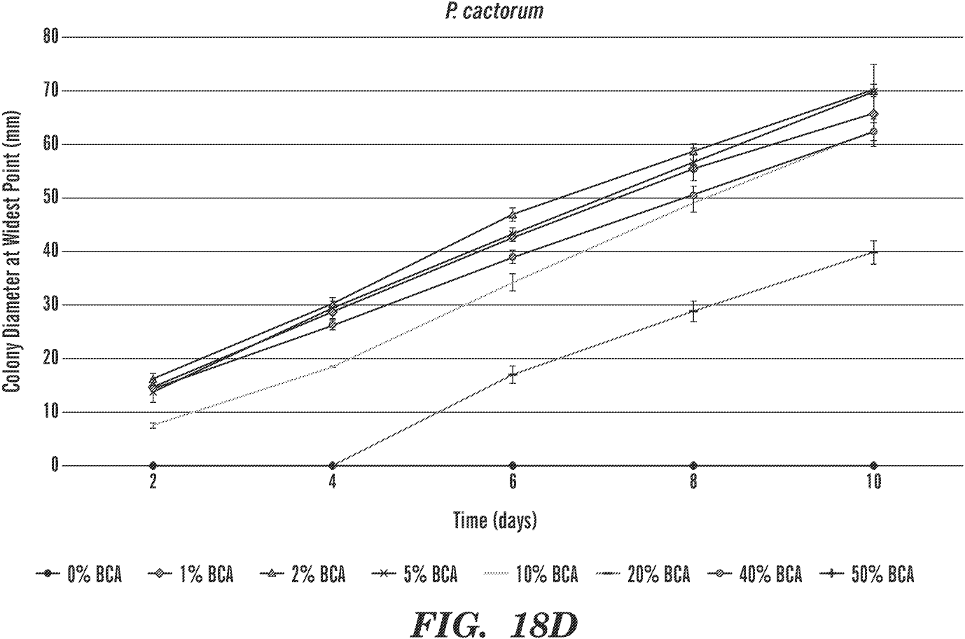

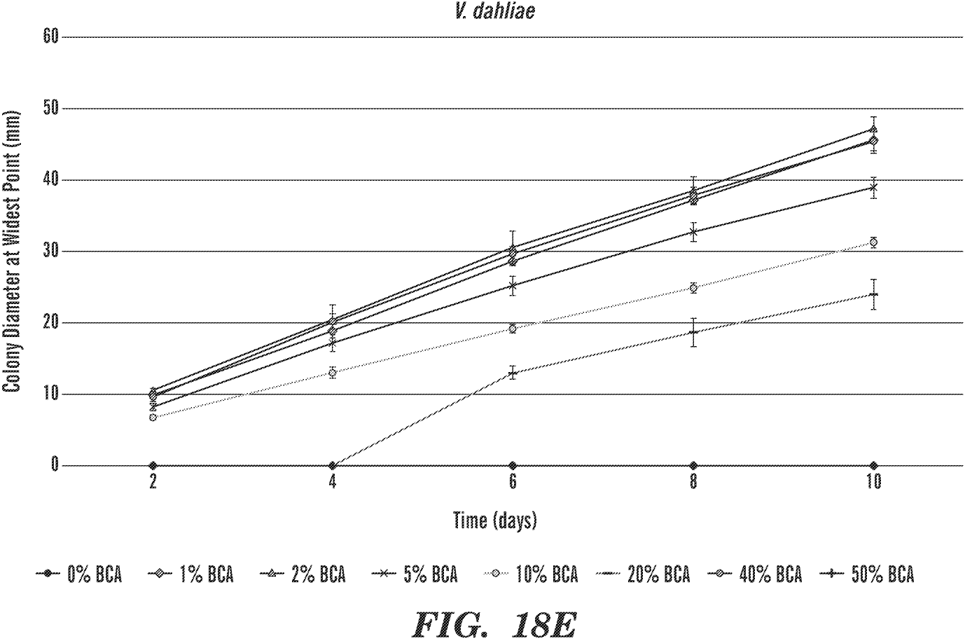

Growth of the fungal pathogens was evaluated (N::::5) on potato dextrose agar (PDA) containing various volumetric concentrations of the biocontrol agent. PDA was prepared with concentrations of the biocontrol agent (v/v) ranging from 0 to 50%. Each of the fungi was grown on PDA containing each of the tested concentrations of the biocontrol agent and colony area was measured over time. The biocontrol agent was capable of inhibiting growth of all of the pathogens evaluated, see FIGS. 18A-18E.

### Example 4. Assessment of Effectiveness of the Biocontrol Agent Against Fungal Pathogens Sclerotinia Minor and Sclerotinia Sclerotiorum in Romaine Lettuce

Having established the efficacy of the biocontrol agents in vitro, the efficacy of the biocontrol agent in controlling growth of plant fungal pathogens was then evaluated in field trial plots of romaine lettuce. It was found that the biocontrol agent was effective in controlling growth of the pathogens.

When a biocontrol agent produced according to the method detailed in Example 1 was applied to the base of the plants, then via drip, the biocontrol agent was as effective as the grower standard (Endura and Cannonball) for controlling Sclerotinia (also known as “lettuce drop” or “white mold”) and enhancing yields of romaine lettuce from plots.

Control of the fungal pathogens, Sclerotinia minor (S. minor) and S. sclerotiorum, in lettuce plots using the biocontrol agent was evaluated. Three application methods (A-C) were evaluated: A) A solo at-planting band spray (1 application; two 6″ bands sprays), B) An at-planting base spray (two 6″ band sprays) followed by a plant base spray at 14 days (2 applications), C) Plant base sprays at 10, 14, and 28 days followed by a drip application at 42 days (4 applications). A 5-application rotational program of industry standard agrochem (Endura® and Cannonball®) was evaluated as a positive control and the application of no treatment was used as a control. The positive control involved applying Endura as two 6″ bands before planting, applying Cannonball by basal spray at 14 days after transplant, applying Endura as a basal spray at 28 days after transplant, applying Cannonball as a basal spray at 35 days after transplant, and applying Cannonball as a basal spray 42 days after transplant. Base sprays were carried out using a backpack CO2 sprayer 4″ from target. Compositions were applied to the soil using a hand boom incorporating 2 TeeJet 8050 nozzles on the outer side drops and 4 TeeJet 8020 nozzles on the inner drops.

The biocontrol composition was applied at an equivalent rate of 2616 gal/acre, Endura (70% wettable granules; 70 WG) was applied at 9 oz wt/acre, and Cannonball (50% wettable powder; 50 WP) was applied at 7 oz wtlacre.

Inferno variety romaine lettuce was transplanted (12″ plant spacing and 2 plant lines per bed with a bed width of 3.33’) Jun. 28, 2019 into clay loam soil in San Luis Obispo, CA. Soil was inoculated with S. minor (5-6 Sclerotia/100 cc) and S. sclerotiorum (4-5 Sclerotia/1000 cc) prior to planting. Plant response, losses to head death, and yields were recorded. The plants were irrigated using the drip method. The romaine lettuce was transplanted into plots with a field spacing equivalent of 3.33’ x 33′. For each treatment N=6. Drip irrigation was used to water the plants. The field spacing equivalent of the plots was 3.33’ x 33′, the soil pH was 8, the soil cation exchange capacity (CEC) was 34.3, the soil % organic matter (OM) was 3.2, the % sand was 20, the % silt was 28, and the % clay was 52.

No plant injury was observed and vigor was uniformly good. Remote sensing (RapidSCAN) readings were not significantly different among treatments for canopy density (Normalized Difference Vegetative Index) and greenness (Normalized Difference Red Edge). None of the treatments resulted in any observed toxicity or any decrease in plant vigor. In measures of overall plant health, none of the treatments displayed symptoms of toxicity, nor displayed meaningful decreases in plant vigor:

Living and dead head counts per plot were taken weekly beginning July 3: The living head counts were highest for plots treated with Endura and Cannonball (only 18 dead). More frequent applications of the biocontrol agent, especially after transplant, resulted in fewer dropped heads due to Sclerotinia. Two applications of the biocontrol agent resulted in 25 dropped heads. Four applications of the biocontrol agent resulted in only 20 dropped heads. There were significantly more dead heads in plots treated with the biocontrol agent only at-planting only and in plots receiving no treatment than in plots receiving more treatments with the biocontrol agent.

Lettuce was harvested by size, with large, medium and small heads counted and weighed per plot, on Aug. 19, 2019. The Endura + Cannonball and the four applications of the biocontrol agent resulted in the most large-sized heads.

Applying the biocontrol agent as multiple basal sprays, and via drip resulted in stand losses on par with the grower standard program of Endura and Cannonball (see Tables 4-9).

TABLE 4

Average Living Head Count - 33 row-ft. Throughout the tables “Trt” means “treatment”.

| Trt No. | Treatment Name | Jul. 3, 2019 Living Heads | Aug. 15, 2019 Living Heads |
|---|---|---|---|
| 1 | Untreated Control | 67.3 | 41.5 |
| 2 | Endura (AC) Cannonball (CDE) | 67.5 | 50.5 |
| 3 | BCA (Application Method A) | 67.8 | 37.8 |
| 4 | BCA (Application Method C) | 67.7 | 49.7 |
| 5 | BCA (Application Method B) | 67.8 | 42.5 |

TABLE 5

Average Dead Head Count - 33 row-ft

| Trt No. | Treatment Name | Jul. 11, 2019 Dead Heads | Aug. 15, 2019 Dead Heads |
|---|---|---|---|
| 1 | Untreated Control | 0.7 | 27.0 |
| 2 | Endura (AC) Cannonball (CDE) | 0.7 | 18.8 |
| 3 | BCA (Application Method A) | 0.2 | 30.3 |
| 4 | BCA (Application Method C) | 0.3 | 20.8 |
| 5 | BCA (Application Method B) | 0.8 | 25.7 |

TABLE 6

Percent Stand Loss

| Trt No. | Treatment Name | PERCENT STAND LOSS |
|---|---|---|
| 1 | Untreated Control | -38.3% |
| 2 | Endura (AC) Cannonball (CDE) | -25.2% |
| 3 | BCA (Application Method A) | -44.3% |
| 4 | BCA (Application Method C) | -26.7% |
| 5 | BCA (Application Method B) | -37.3% |

TABLE 7

Yield Counts per Plot. Large (18 s), Medium (24 s), Small (36 s)

| Trt No. | Treatment Name | YIELD COUNT P ER PLOT | | |
|---|---|---|---|---|
| | | LARGE | MEDIUM | SMALL |
| 1 | Untreated Control | 8.2 | 14.5 | 9.8 |
| 2 | Endura (AC) Cannonball (CDE) | 14.0 | 11.0 | 16.0 |
| 3 | BCA (Application Method A) | 3.8 | 12.8 | 12.0 |
| 4 | BCA (Application Method C) | 11.7 | 15.2 | 14.7 |
| 5 | BCA (Application Method B) | 5.8 | 16.0 | 14.0 |

TABLE 8

Yield Weight per Plot (kilograms)

| Trt No. | Treatment Name | |
|---|---|---|
| 1 | Untreated Control | 27,875.3.00 |
| 2 | Endura (AC) Cannonball (CDE) | 32,732.1.00 |
| 3 | BCA (Application Method A) | 22,071.7.00 |
| 4 | BCA (Application Method C) | 33,123.9.00 |
| 5 | BCA (Application Method B) | 27,197.8.00 |

TABLE 9

Yield Composition (by weight)

| Trt No. | Treatment Name | YIELD COMPOSITION | | |
|---|---|---|---|---|
| | | LARGE | MEDIUM | SMALL |
| 1 | Untreated Control | 35.5% | 42.6% | 21.9% |
| 2 | Endura (AC) Cannonball (CDE) | 32.3% | 29.6% | 38.1% |
| 3 | BCA (Application Method A) | 17.4% | 44.6% | 38.0% |
| 4 | BCA (Application Method C) | 35.5% | 36.6% | 27.9% |
| 5 | BCA (Application Method B) | 19.5% | 44.6% | 36.0% |

### Example 5. Assessment of Effectiveness of the Biocontrol Agent Against the Fungal Pathogen Sclerotinia Sclerotiorum in Romaine Lettuce

The efficacy of the biocontrol agent in controlling growth of a fungal pathogen was again evaluated in plots of romaine lettuce. Results confirmed that the biocontrol agent was effective in controlling growth of the pathogen. Plant losses were greatest in untreated plots. There were more dead heads counted in plots treated with just one application of the biocontrol agent than in plots treated with two or five applications of the biocontrol agent. The marketable yield of romaine lettuce was equivalent between plots treated with the biocontrol agent and a rotational program of industry standards Endura® and Cannonball®.

Green Thunder and Carbine varieties of romaine lettuce were transplanted on Sep. 2, 2019 in a sand/silt/clay (35% / 28% / 37%) soil at the Riverside Ranch farm of Pacific Ag Research in Spreckels, CA. Soil was inoculated with S: sclerotiorum. The plots had a field spacing equivalent of 35′ x 3.33’. Plant spacing was 12″ and bed width was 40″. Drip irrigation was used to water the plants. The soil pH was 8.5, the soil CEC (cation exchange capacity) was 29.5, and the soil organic matter (OM) was 2.4%. For each treatment N=6.

Control of the soil pest S. sclerotiorum in romaine lettuce plots using the biocontrol agent was evaluated using three different application methods: (×1) A solo at-planting band spray (1 application); (x2) An at-planting base spray and a plant base spray at 14 days (2 applications); (x5) Plant base sprays at planting and at 14, 30, and 36 days post-planting followed by a drip application at 42 days post-planting (5 applications).

As in Example 4, for comparison, plots treated with a 5-application rotational program of industry standard agrochem (Endura® and Cannonball®) were evaluated, and plots receiving no treatment at all were also were evaluated. For the 5-application rotational program of industrial standard agrochem, Endura and Cannonball were applied as described in Example 4 above.

The biocontrol composition was applied at 2616 gal/acre, Endura (70% wettable granules; 70 WG) was applied at 9 oz wt/acre, and Cannonball (50% wettable powder; 50 WP) was applied at 7 oz wt/acre.

The biocontrol agent, Endura, and Cannonball were applied to the plots using a backpack CO2 sprayer at 40 psi operating pressure or a tractor mounted fertilizer boom at 60 psi operating pressure Drip application was applied to the root zone at 10 psi operating pressure.

Plant response, losses to head death, and yields were recorded. No plant injury was observed, and vigor ratings were generally uniform. One week after the first post-plant spray application, the agrochem standard-treatment lettuce was rated least vigorous. By the end of the trial, the untreated plants were least vigorous, statistically. RapidSCAN remote sensing equipment was used to measure canopy greenness and density. The industry standard agrochem increased canopy greenness and density relative untreated plants.

Stand counts were recorded weekly for each plot. The living lettuce counts were compared with dead heads resulting from lettuce drop, which is the disease caused by Sclerotinia sclerotiorum. Overall losses were greatest in untreated plots There were more dead heads counted in the plots treated with just one application of the biocontrol agent than in plots treated with two or five applications. In summary:

Untreated plots yielded a living head count average of 40.7 . Living head count averages for plots were identical (47.3) for 5 applications of Endura-Cannonball and 5 applications of the biocontrol agent. Two applications of the biocontrol agent resulted in a living head count average of 46.8

As demonstrated in Example 4, applying the biocontrol agent as multiple basal sprays, and via drip resulted in stand losses on par with the grower standard program of Endura and Cannonball.

Lettuce was harvested Nov. 12, 2019, and counts and weights of small, medium, large and unmarketable heads were recorded. Larges were 11-12″ long, medium were 9-11″, smalls were <8″, while culls were heads damaged by disease, misshapen, discolored, or otherwise unfit for market. There were significantly more large-sized heads collected from plots treated with two applications of Tu Biomics extract, and the fewest large heads were counted in plots treated with 5 Tu Biomics applications. Yield composition was not significantly different for the treatments and total yields per acre of marketable cartons of 12 3-heart packages were higher for the treated plots, numerically.

Untreated controls yielded 334 cartons on average, for an estimated market value of $11,354. The Endura-Cannonball standard treatment yielded 392 cartons, with market value of $13,355. All plots treated with the biocontrol agent yielded improved averages over untreated controls. One application of the biocontrol agent yielded an average of 377 cartons per acre, with market value of $12,825. Two applications of the biocontrol agent yielded an average of 392 cartons per acre, with market value of $13,355. Five applications of the biocontrol agent yielded an average of 398 cartons per acre, with market value of $13,531.

Tables 11-18 present average living/dead head counts, percent stand loss, powdery mildew severity, and yields.

TABLE 11

Average Living Head Count.

| Trt No. | Treatment Name | Sep. 10, 2019 Living Heads | Nov. 11, 2019 Living Heads |
|---|---|---|---|
| 1 | Untreated Control | 66.3 | 40.7 |
| 2 | Endura (AC) Cannonball (CDE) | 66.3 | 47.3 |
| 3 | Biocontrol agent (×1) | 67.8 | 46.5 |
| 4 | Biocontrol agent (x5) | 67.3 | 47.3 |
| 5 | Biocontrol agent (x2) | 68.3 | 46.8 |

TABLE 12

Average Dead Head Count.

| Trt No. | Treatment Name | Sep. 10, 2019 Dead Heads | Nov. 11, 2019 Dead Heads |
|---|---|---|---|
| 1 | Untreated Control | 0.5 | 23.5 |
| 2 | Endura (AC) Cannonball (CDE) | 1.2 | 17.3 |
| 3 | Biocontrol agent (×1) | 0.2 | 18.0 |
| 4 | Biocontrol agent (x5) | 0.3 | 16.7 |
| 5 | Biocontrol agent (x2) | 0.0 | 19.5 |

TABLE 13

Percent Stand Loss.

| Trt No. | Treatment Name | PERCENT STAND LOSS |
|---|---|---|
| 1 | Untreated Control | -36.6% |
| 2 | Endura (AC) Cannonball (CDE) | -26.4% |
| 3 | Biocontrol agent (x 1) | -27.9% |
| 4 | Biocontrol agent (x5) | -26.2% |
| 5 | Biocontrol agent (x2) | -29.3% |

TABLE 14

% Severity Powdery Mildew at Harvest. Severity was assessed by visual inspection and count by an agronomist.

| Trt No. | Treatment Name | Nov. 11, 2019 Severity of Powdery Mildew |
|---|---|---|
| 1 | Untreated Control | 66.9% |
| 2 | Endura (AC) Cannonball (BDE) | 70.3% |
| 3 | Biocontrol agent (x1) | 67.8% |
| 4 | Biocontrol agent (x5) | 74.2% |
| 5 | Biocontrol agent (x2) | 71.9% |

TABLE 15

Yield Counts per Plot.

| Trt No. | Treatment Name | YIELD COUNT PER PLOT | | | |
|---|---|---|---|---|---|
| | | CULLS | LARGE | MEDIUM | SMALL |
| 1 | Untreated Control | 30.8 | 27.7 | 3.7 | 0.8 |
| 2 | Endura (AC) Cannonball (CDE) | 26.2 | 32.7 | 4.3 | 0.8 |
| 3 | Biocontrol agent (×1) | 29.2 | 28.8 | 6.8 | 0.7 |
| 4 | Biocontrol agent (x5) | 28.7 | 25.2 | 11.5 | 1.7 |
| 5 | Biocontrol agent (x2) | 29.3 | 33.7 | 3.7 | 0.5 |

TABLE 16

Yield Weight per Plot (LBS). Green thunder and carbine are the two varieties of romaine lettuce that were grown in the experiment.

| Trt No. | Treatment Name | GREEN WEIGHT | | THUNDER / CARBINE | |
|---|---|---|---|---|---|
| | | CULLS | LARGE | MEDIUM | SMALL |
| 1 | Untreated Control | 12.6 | 49.0 | 4.6 | 0.9 |
| 2 | Endura (AC) Cannonball (CDE) | 14.2 | 54.3 | 5.1 | 0.8 |
| 3 | Biocontrol agent (×1) | 13.2 | 44.8 | 8.8 | 0.7 |
| 4 | Biocontrol agent (x5) | 12.5 | 43.2 | 13.1 | 1.6 |
| 5 | Biocontrol agent (x2) | 14.1 | 62.7 | 4.6 | 0.6 |

TABLE 17

Percent Yield by Weight.

| Trt No. | Treatment Name | YIELD COMPOSITION | | | |
|---|---|---|---|---|---|
| | | CULLS | LARGE | MEDIUM | SMALL |
| 1 | Untreated Control | 19.1% | 72.1% | 7.2% | 1.6% |
| 2 | Endura (AC) Cannonball (CDE) | 19.1% | 72.3% | 7.4% | 1.2% |
| 3 | Biocontrol agent (x 1) | 19.9% | 65.6% | 13.5% | 1.0% |
| 4 | Biocontrol agent (x5) | 17.8% | 59.0% | 20.8% | 2.5% |
| 5 | Biocontrol agent (x2) | 17.3% | 76.3% | 5.7% | 0.7% |

TABLE 18

Yields per Acre - 12 3-heart Pkgs per Carton.

| Trt No. | Treatment Name | Cartons Per Acre | Estimated $/Acre |
|---|---|---|---|
| 1 | Untreated Control | 334.0 | $11,354 |
| 2 | Endura (AC) Cannonball (CDE) | 392.8 | $13,355 |
| 3 | Biocontrol agent (x 1) | 377.2 | $12,825 |
| 4 | Biocontrol agent (x5) | 398.0 | $13,531 |
| 5 | Biocontrol agent (x2) | 392.8 | $13,355 |

$/Acre based on a value of $34/carton per USDA market report.

### Example 6. Efficacy of the Biocontrol Agent in Controlling Growth of Sclerotium Cepivorum in a Garlic Culture

Efficacy of the biocontrol agent produced according to the methods exemplified in Example 1 in the control of white rot (Sclerotium cepivorum) in a garlic plant culture was evaluated in a study.

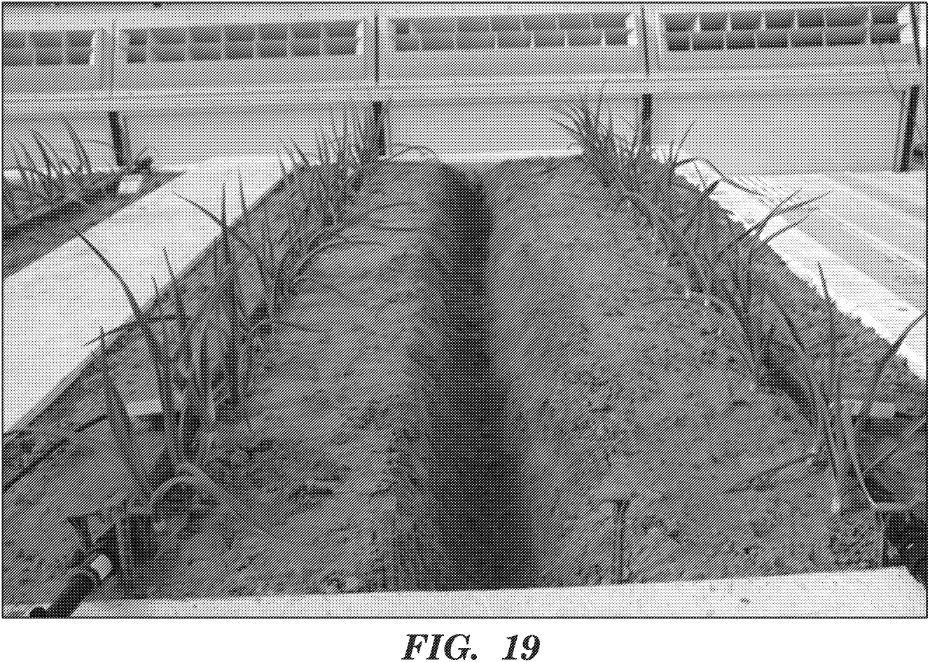

One of the Ecologically Controlled Lysimeters (EcoCELL) located at the Desert Research Institute’s (DRI’s) Reno campus was used for the study. There were three pots within the EcoCELL, each with dimensions of 2.4 m (8 ft) x 1.2 m (4 ft) x 1.8 m (6 ft). The top 40 cm of each of two of the three pots (north and middle pots) was filled with soil that was collected from a garlic field in Yerington, Nevada that had become infected with white rot (Sclerotium cepivorum). The top 40 cm of the third pot (south pot) was filled with healthy soil (no white sclerotia present) from a nearby garlic field. Each of the three pots within the EcoCELL contained two beds (FIG. 19). The topsoil in all pots was place on ∼150 cm deep layer of well drained silt-loam soil removed intact from a tailgrass prairie site in central Oklahoma. Garlic was planted in the EcoCELL on Nov. 2, 2018. Garlic cloves were placed one inch below the soil surface with four inches between each clove within a seedline resulting in 24 planted cloves per 2.4 m seed line Three of the planted cloves in each seed line did not develop into plants.

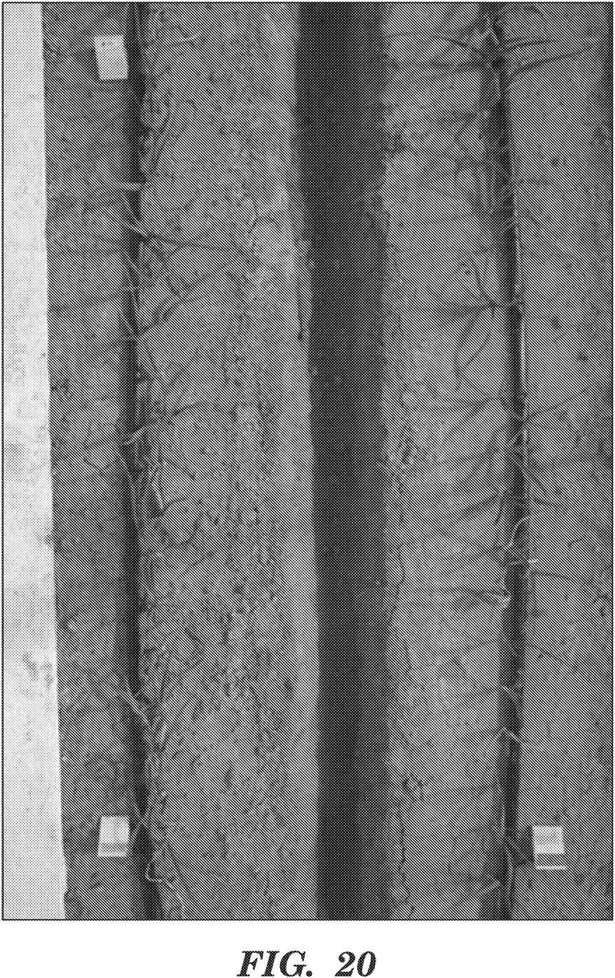

Irrigation drip tape (NETAFIM Streamline Plus) was installed on the surface of each bed within a pot (FIG. 20). Drip emitters embedded within the drip tape were spaced every eight inches and released 0.18 gallons/hour. Irrigation was controlled by a programmable and automated irrigation system. The drip tape supplied water only. The biocontrol agent treatment was applied as a liquid with a watering can at a rate of 2 gal per 10 ft of bed.

Temperature and relative humidity within the EcoCELL during the 8.5 month study mimicked average diel and seasonal conditions of the San Juan Bautista, California field site.

Volumetric soil water content was measured in two locations within each bed with CS616 TDR sensors. Sensors were installed at an angle in order to represent the average volumetric soil water content from 0 to 20 cm (8 inches) deep. These sensors were used principally to indicate when soils required irrigation and how much water to apply, but also were used to evaluate water or biocontrol agent infiltration into the soil profile.

The north bed within each pot were treated with the biocontrol agent and the south bed within each pot was treated only with water as a negative control. The biocontrol agent was applied with a watering can on 24 days throughout the study. At the time of biocontrol agent application, an equal amount of water was applied to the water treatment beds (experimental control) with a watering can. All beds were also irrigated with tap water applied through the drip tape.

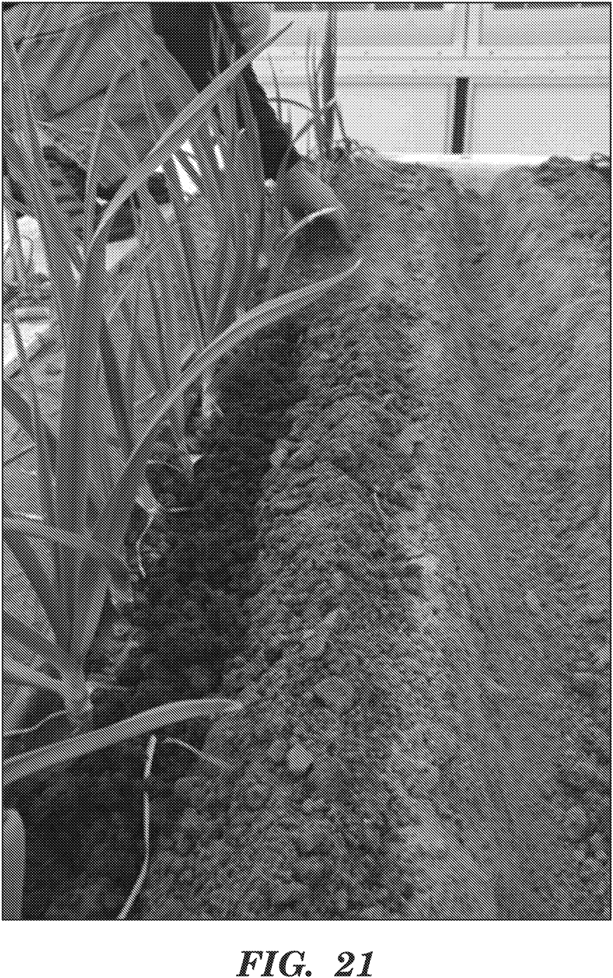

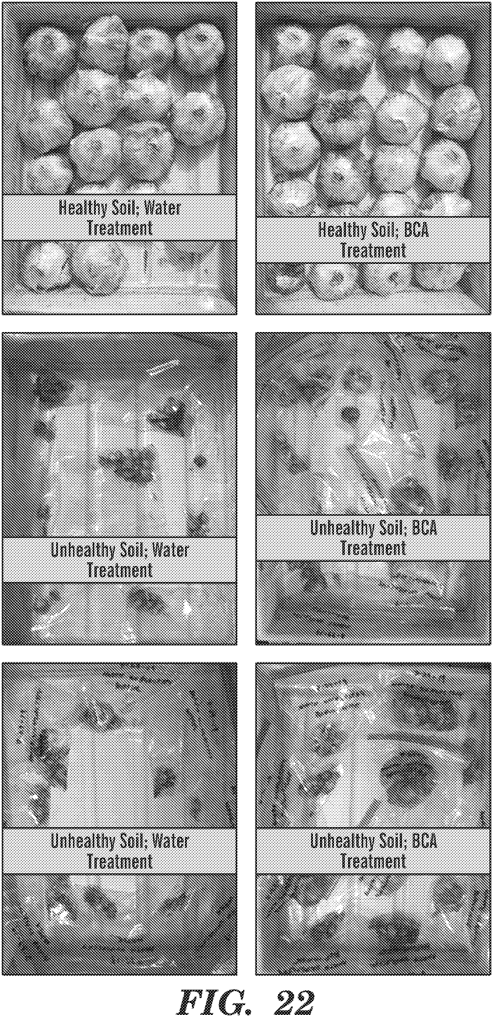

To ensure soil was infected with white rot, all beds within the unhealthy soil pots were inoculated with soil from San Juan Bautista that had been confirmed to have high levels of white rot infection. On Mar. 20, 2019 the infected soil was applied along the length of each bed and adjacent to the garlic plants (FIG. 21).

Irrigation was stopped on Jun. 16, 2019 to allow soil and bulbs to dry. On Jul. 17, 2019 all garlic plants were pulled from the soil, laid on the bed surface, and allowed to cure for 12 days under ambient EcoCELL atmospheric conditions. On Jul. 29, 2019, shoots and roots were cut from the garlic bulbs. Bulbs from all beds and treatments were photographed (FIG. 22) and individually weighed.

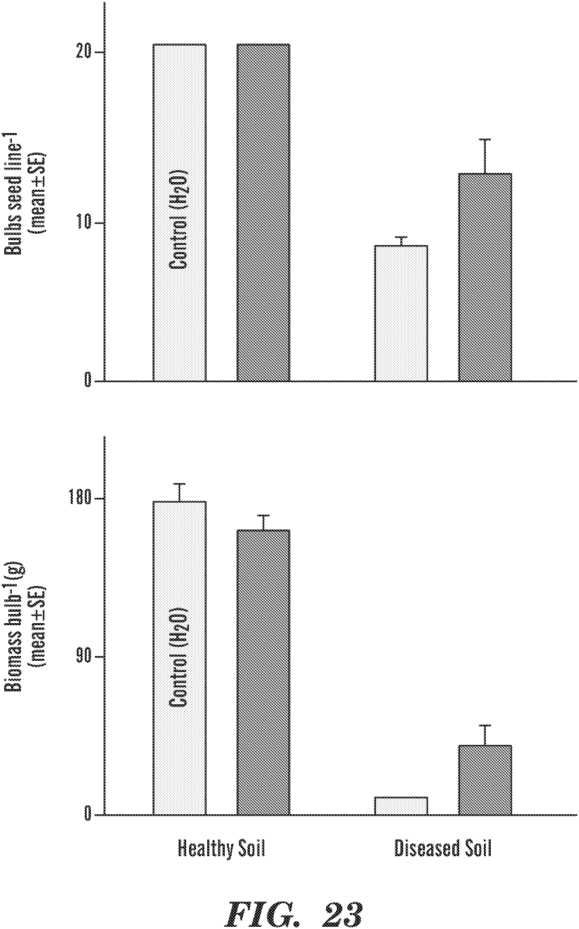

Garlic plants developed normally in both the control (water applied) and biocontrol agent-treated seed lines when grown in healthy Yerington field soil, with bulbs very similar in dimensions and mass to bulbs produced on plants growing in the field (FIG. 22). In healthy soil, application of the biocontrol agent had no effect on mean bulb mass at harvest (P=0.2141, not statistically significant [n.s.]) with a mean bulb mass of 176±10 g per bulb (n=17) for plants treated with the water control and 160±8 g per bulb (n=21) for plants treated with the biocontrol agent (FIG. 23). Four plants treated with the water control were harvested during the study to evaluate bulb development and cloving, which accounted for the lower number of bulbs available at harvest relative to plants treated with the biocontrol agent where no bulbs were sampled before final harvest. The slightly greater mean bulb mass in the controls may have been due to a stimulatory effect of thinning on the remaining water-treated plants.

Treatment with the biocontrol agent noticeably slowed the development of white rot symptoms (FIG. 22). At harvest, this was evident by the presence of a few green and less-effected plants remaining in the seed lines treated with the biocontrol agent, relative to that observed in the seed lines treated with water. Further evidence of the effectiveness of the biocontrol agent was seen in the greater mean number of bulbs in the biocontrol agent-treated seed lines (13±2 bulbs per seed line, n=2 seed lines; P=0.2048, n.s.) than in the seed lines treated with water (8.5±0.5 bulbs per seed line, n=2) (FIG. 23) Similarly, mean biomass per bulb in seed lines treated with the biocontrol agent was 38.9±10.4 g bulb⁻¹, and in controls treated with water the mean biomass per bulb was 8.71±0.06 g bulb⁻¹ (P=0.2105, n.s.; even though these were not statistically discernable with n=2 pots) (FIG. 23). These differences represented a nearly four-fold increase in mean bulb mass and a 53% increase in the number of bulbs per seed line through treatment with the biocontrol agent. For mass per bulb, this result was statistically significant when using “plant” as the statistical unit (P=0.0479 for the “north EcoCELL pot”, and P=0.0214 across the north and middle EcoCELL diseased soil pots) (FIG. 23). Further, mean bulb mass was dramatically lower in diseased soil than in healthy soil, and this reduction was limited to a 4-fold reduction in seed lines treated with the biocontrol agent, as compared to a 20-fold reduction in bulb biomass in seed lines treated with water. Also, three nearly-healthy plants that produced normal-sized bulbs were observed growing in diseased soil treated with the biocontrol agent seed.

Application of the biocontrol agent to plants growing in healthy soil neither reduced nor stimulated plant or bulb growth, which suggests that the beneficial effect observed in plants growing in diseased soil was likely due to a direct effect on pathogen itself to alterations in the pathogen-plant relationship and not due to a fertilizer/nutrient effect.

In summary, application of the biocontrol agent to diseased soils in which garlic plants were growing (1) significantly delayed the onset of white rot symptoms when garlic was grown in diseased soil; (2) mitigated the damage done by the presence of white rot in terms of plant health; (3) increased the number of garlic plants that formed bulbs; and (4) greatly increased the growth of bulbs on plants that formed bulbs.

### Example 7. Trial to Assess the Efficacy of the Biocontrol Agent for White Rot Disease Control in Garlic

The plant disease white rot is caused by the fungal pathogen Sclerotium cepivorum. This disease is a devastating disease that affects plants in the Allium Family (Garlic, Onions, and Shallots). Plants can become infected in any stage of the growth cycle depending on the soil temperature. Moist and cool soil conditions are favorable for disease development. The range of optimum soil temperatures for development of white rot are of 50° to 75° F. When soil temperatures are above 78° F., the disease is inhibited in the soil. When initial disease development occurs on a garlic plant, a fluffy white growth (fungal mycelium) is observable at the base of the garlic bulb. Mycelium feeding causes the roots and bulb to rot and decay. When the mycelium becomes more compacted, the fungi tends to form multiple small black dormant structure known as sclerotia. The sclerotia can remain dormant in the soil until there is a suitable host. The sclerotia can remain dormant in the soil for as long as 20 years. In many instances, the fungal pathogen is transferred from an infected field into a non-infected field via contaminated soil. Sclerotia can travel via agricultural machinery and it only takes a few grams of soil to carry sclerotia into a neighboring field, thereby contaminating the field. Avoidance and sanitation are very important in the mitigation of the disease.

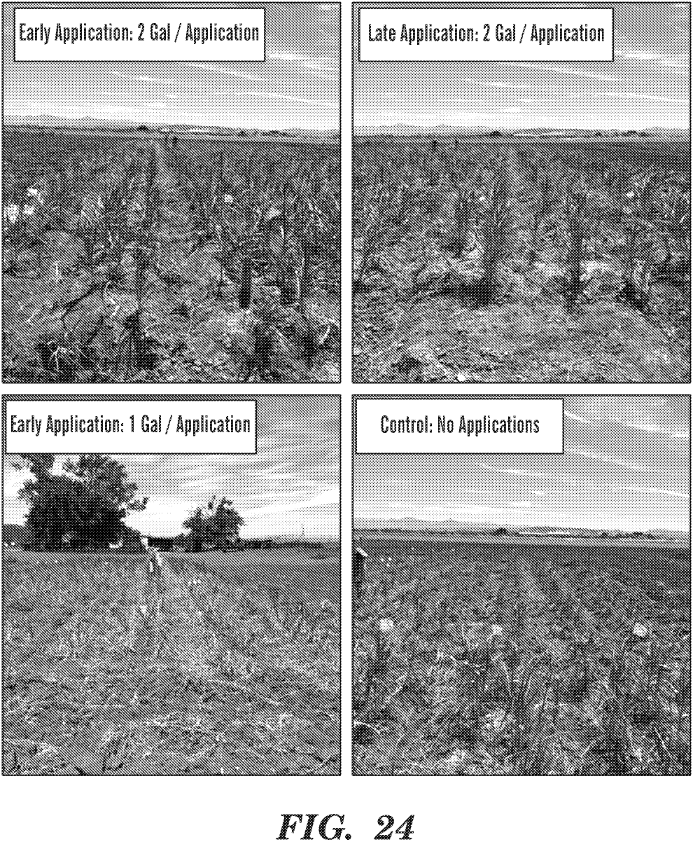

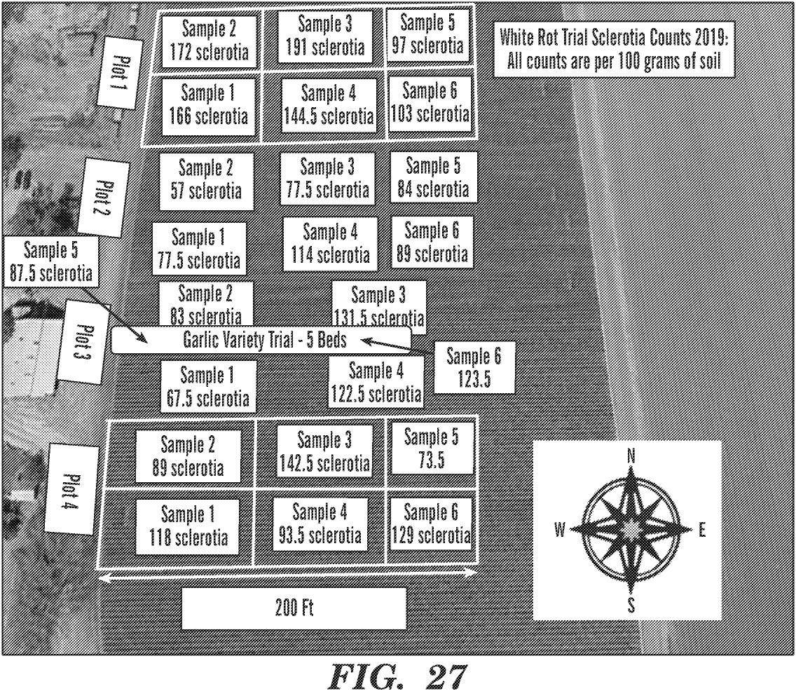

The trial was conducted in San Juan Bautista, CA during the growing season of 2018- 2019. During the growing season of 2017-2018, an organic garlic field located in San Juan Bautista was declared 100% white rot infected. Due to this, a total of two acres where isolated from this field to do the trials during the growing season of 2018-2019 (FIG. 27). The soil type for this trial was a Sorrento silt loam (99.6%) and Sorrento silty clay loam (0.4%). Prior to planting, soil samples were taken throughout the two acres to calculate how many sclerotia were present. The two acres of trial where broken down into four test plots (FIG. 24). Each test plot in the trial was sampled in six different areas. Soil samples were placed in labeled bags. The samples were evaluated in a pathologist lab, where the sieving method was used to obtain sclerotia counts per 100 grams of soil. A map was created to map disease inoculum present in the different areas sampled (FIG. 27).

The trial was planted on Nov. 20, 2018 using a standard industrial garlic planter. The garlic variety that was used is the California late. The garlic was planted at a rate of 14 cloves per bed foot After planting, two pre-emergence herbicides where applied in a tank mix for pre-emergent weed control. The herbicides used where Chateau SW (60 oz/ Ac) and Prowl H20 (2 pt / Ac). The herbicides were applied in a broadcast spray using a tractor at a rate of 25 Gallons of water/ Ac. According to California Irrigation Management Information System (CIMIS), the total cumulative rainfall for the trial season was 12.88 inches. The method of irrigation was a sprinkler irrigation system. Irrigation was for about 5 hours/ week. Irrigation needs where measured using a John Deere Field connect moisture probe irrigations.

To prevent the results from being impacted by plant infection by garlic rust (Puccinia allii), three applications of a fungicide specific to this fungal pathogen were applied to the plants. The first fungicide application took place Mar. 15, 2019. The fungicide applied was Quadris (12 Fl Oz/ Ac) and a Multi-spread adjuvant (1 Pt/ Ac). On April 12 the same products were re-applied. The final application took place on Apr. 29, 2019 and different fungicides where applied. The fungicides applied were Fontelis (24 Fl Oz/ Ac), Tebuzol 3.6 F (4 Fl Oz/ Ac) and Multi spread (1 Pt/ Ac) The fungicides applied were selected to avoid any impact on white rot. No fertilizers or insecticides were added to the trial.

The compositions used in this trial for white rot disease control where the biocontrol agent and water (a negative control). The layout of the trial for early application of the compositions included four garlic beds with two seed lines / bed. Each of the four garlic beds were divided into two sections. The first section was the first 50 ft from west to east The 50 ft for both sections were broken down into 10 ft on both seed lines for calibration purposes Each of the two sections received a different rate of application of the compositions. In the first section, the rate of applications was 2 gallons per 10 ft, and in the second section, the rate of application was 1 Gallon per 10 ft (Table 19).

TABLE 19

Healthy Plant Percentage of Early Applications. During the experiment, the biocontrol agent may have drifted to adjacent water-only plots.
Early Application Healthy Plant Counts

| Biocontrol Agent: | Date: | Total Plants | Healthy plants | %Healthy Plants |
|---|---|---|---|---|
| Bed 1: 2 Gallons/ 10 Ft application | Jun. 24, 2019 | 593 | 97 | 16% |
| Bed 1: 1 Gallons/ 10 Ft application | Jun. 24, 2019 | 594 | 36 | 6% |
| Water: | Date: | Total Plants | Healthy plants | %Healthy Plants |
| Bed 2: 2 Gallons/ 10 Ft Application | Jun. 24, 2019 | 576 | 167 | 29% |
| Bed 2: 1 Gallons/ 10 Ft Application | Jun. 24, 2019 | 574 | 32 | 6% |
| Biocontrol Agent: | Date: | Total Plants | Healthy plants | %Healthy Plants |
| Bed 3: 2 Gallons/ 10 Ft Application | Jun. 24, 2019 | 513 | 166 | 32% |
| Bed 3: 1 Gallons/ 10 Ft Application | Jun. 24, 2019 | 572 | 67 | 12% |
| Water: | Date: | Total Plants | Healthy plants | %Healthy Plants |
| Bed 4: 2 Gallons/ Appl South Seed Line | Jun. 24, 2019 | 642 | 114 | 18% |
| Bed 4: 1 Gallons/ Appli North Seed Line | Jun. 24, 2019 | 649 | 36 | 6% |

Both the water and the biocontrol agent where first applied on Feb. 22, 2019. White rot disease was already active in the test plot when the first application took place. The compositions were applied using a gardening watering can that sprinkled the material at the base of the plant. A total of 19 gallons / 10 ft was applied to the first section at the rate of 2 gallons / application. A total of 8 Gallons / 10 ft was applied to the second section at the application rate of 1 gallon / ft.

On Apr. 2, 2019 a total of four new beds were added to the trial (“Late Application”) with a layout similar to that described above for the early application. The east most 50 ft of each bed was treated with a rate of application of 2 Gallons / 10 ft. The west most 50 ft of each bed was assigned as a control plot (“Control”) and no water or biocontrol agent was applied to these plots. A total of 9 Gallons / 10 ft of the biocontrol agent or water was applied to the treated plots (Table 20).

TABLE 20

Healthy Plant Percentage of Late Applications.
Later Application Healthy Plant Counts

| Water: | Date: | Total Plants | Healthy plants | %Healthy Plants |
|---|---|---|---|---|
| Bed 5: 2 Gallons/ 10 ft Application | Jun. 24, 2019 | 658 | 95 | 14% |
| Bed 5: Control | Jun. 24, 2019 | 647 | 26 | 4% |
| Biocontrol Agent: | Date: | Total Plants | Healthy plants | %Healthy Plants |
| Bed 6: 2 Gallons/ 10 ft Application | Jun. 24, 2019 | 609 | 115 | 19% |
| Bed 6: Control | Jun. 24, 2019 | 597 | 35 | 6% |
| Water: | Date: | Total Plants | Healthy plants | %Healthy Plants |
| Bed 7: 2 Gallons/ 10 ft Application | Jun. 24, 2019 | 696 | 121 | 17% |
| Bed 7: Control | Jun. 24, 2019 | 687 | 52 | 8% |
| Biocontrol Agent: | Date: | Total Plants | Healthy plants | %Healthy Plants |
| Bed 8: 2 Gallons/ 10 ft Application | Jun. 24, 2019 | 640 | 81 | 13% |
| Bed 8: Control | Jun. 24, 2019 | 632 | 33 | 5% |

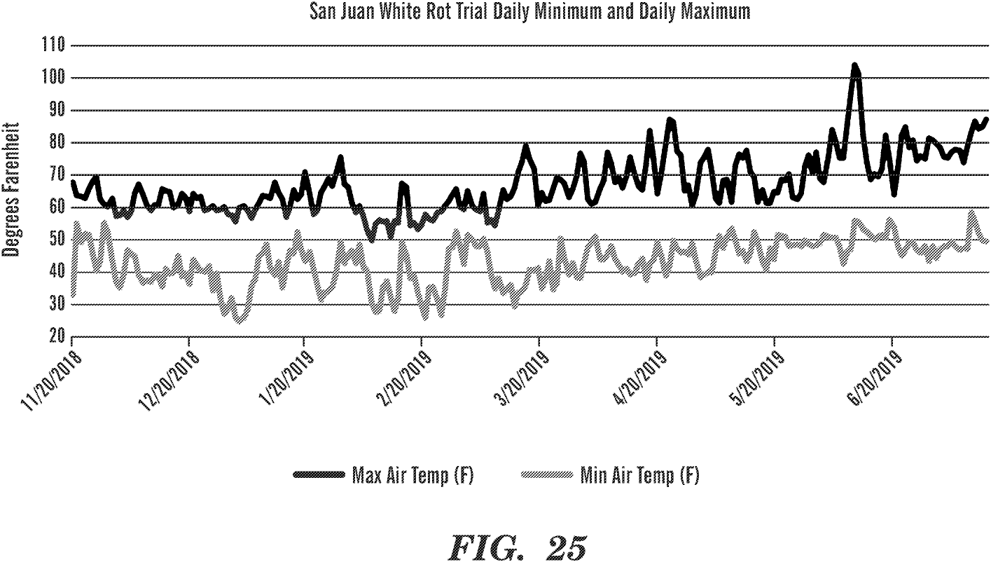

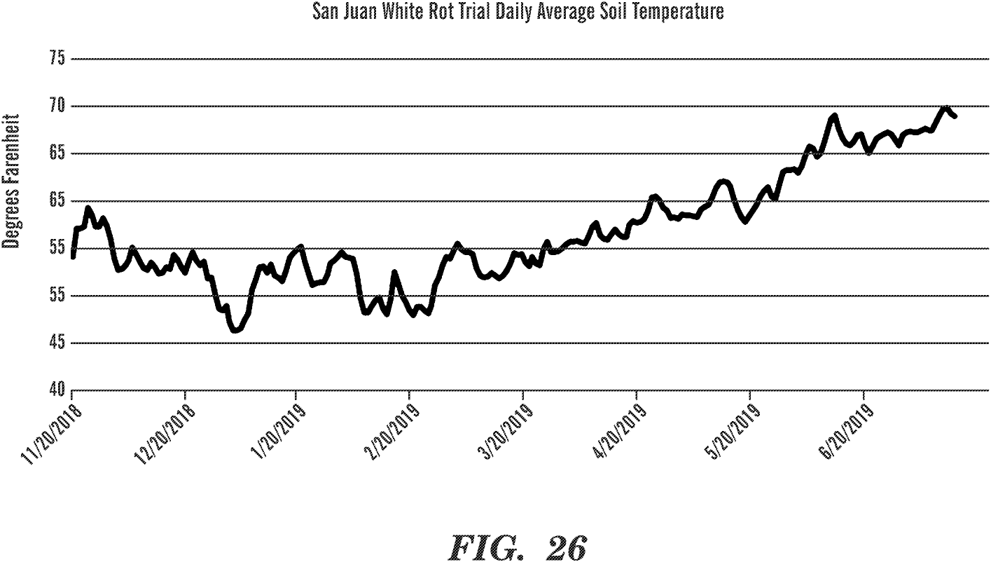

After evaluating the Tu Biomics test plots on Jun. 24, 2019 there was a noticeable difference between the test plots evaluated in the trial (FIG. 24). There was a suppression of number of garlic plants infected with white rot where the biocontrol agent was applied at a rate of 2 Gallons/ 10 ft as compared to the controls and the lower rate of biocontrol agent evaluated. A higher application rate of the biocontrol agent correlated with an increase in garlic plant health. Throughout the trial, the soil temperature was optimal for white rot growth throughout the trial period (FIGS. 25 and 26). The biocontrol was able to suppress the activity of white rot on garlic plants.

### Example 8. Testing of Efficacy of the Biocontrol Agent in Controlling Plant Pathogens

The efficacy of the biocontrol agent in controlling the growth of the agriculturally important fungi Botrytis cinerea, Colletotrichum acutatum, Fusarium oxysporum sp. fragariαe , Macrophomina phaseolina, Phytophora cactorum, Rhizoctonia solani, Sclerotium cepivorum, and Verticillium dahliae was evaluated. Fusarium oxysporum f. sp.fragariae is specialized and causes fusarium wilt of only strawberry. Sclerotium cepivorum also has a narrow host range and causes white rot of allium crops The following four fungi were isolated from infected strawberries: Colletotrichum acutatum, Fusarium oxysporum f. sp. fragariae, Macrophomina phaseolina, and Phytophthora cactorum.

The biocontrol agent or water (the negative control) was mixed with one part potato dextrose agar (PDA) containing streptomycin (example: 180 ml of streptomycin-PDA mixed with 180 ml of the biocontrol agent). Petri plates prepared using the PDA mixtures were cooled and then inoculated with fungi the same day that they were prepared. Plates were inoculated with a single 5-mm diameter agar plug containing the fungus to be treated. The plugs were placed in the middle of each petri plate. Each treatment was evaluated in triplicate. Inoculated plates incubated at room temperature in a darkened incubator.

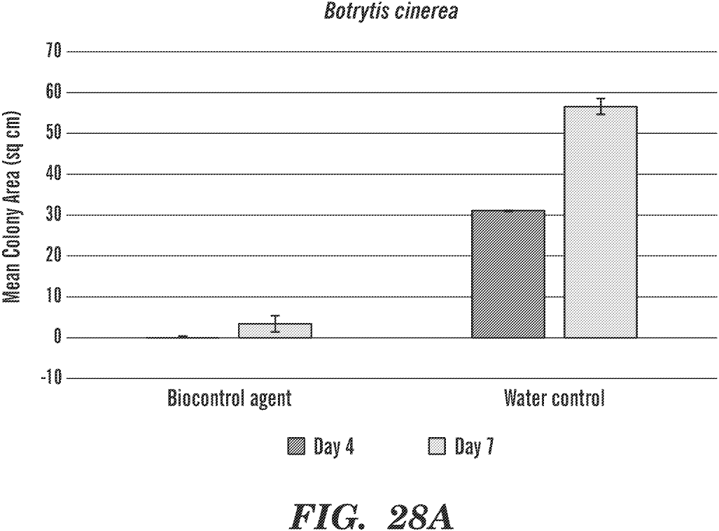

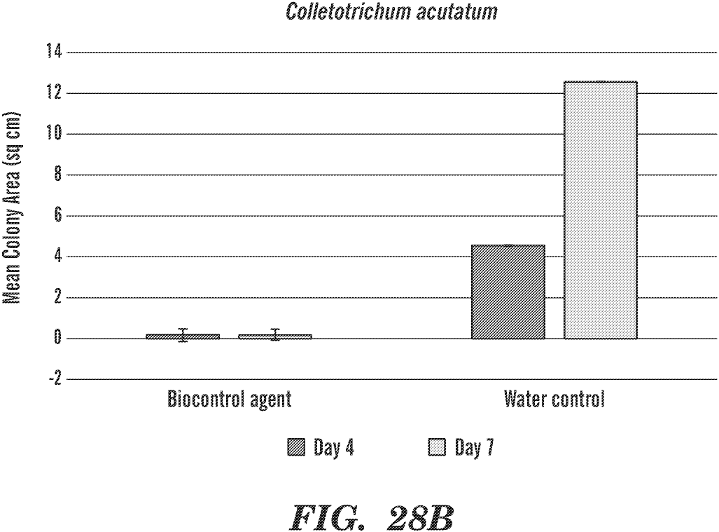

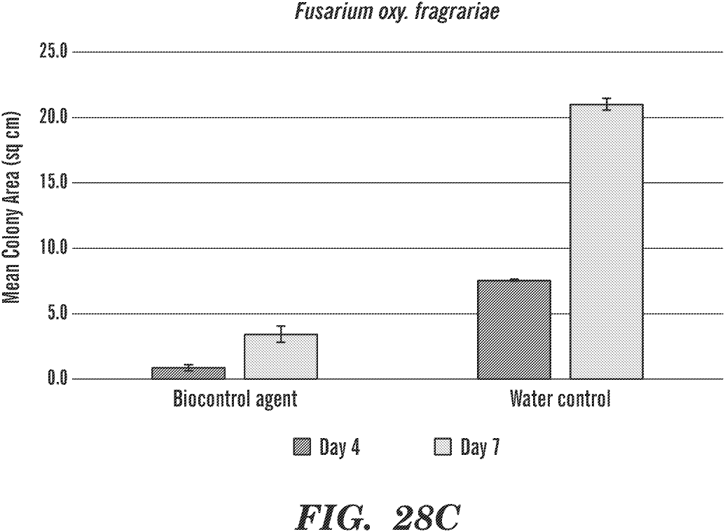

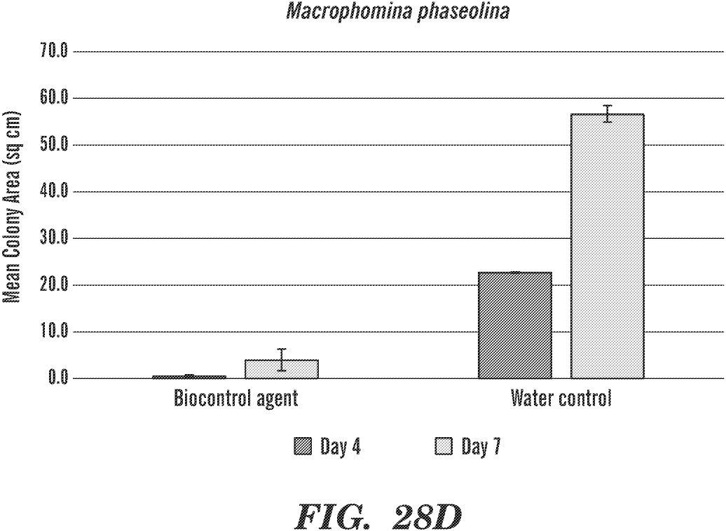

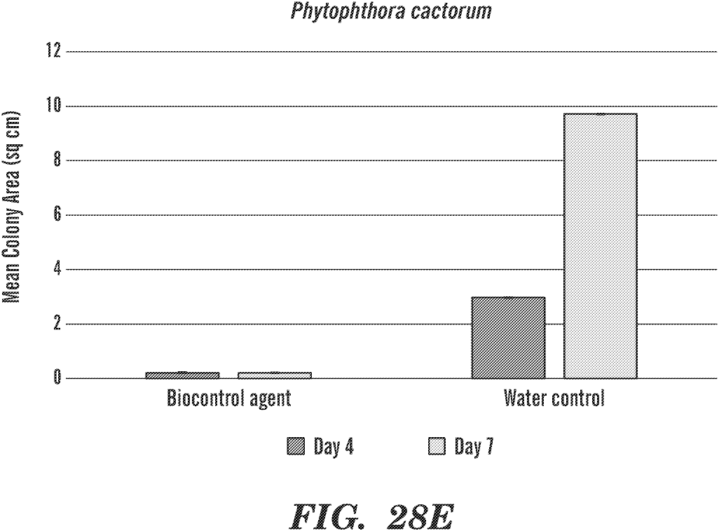

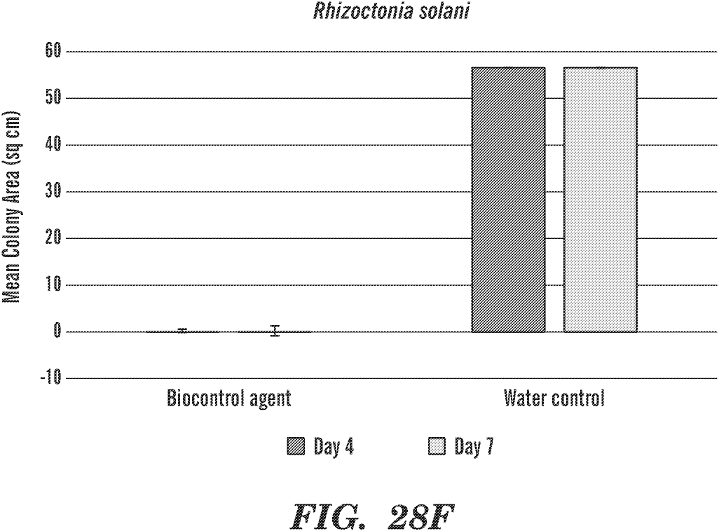

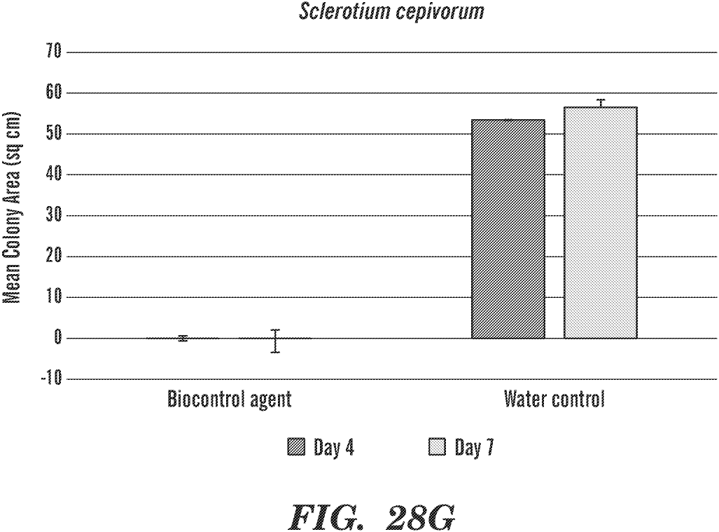

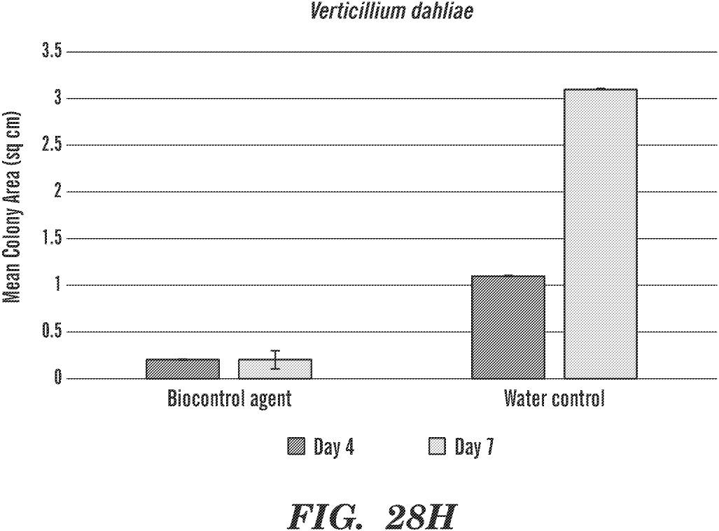

Data on fungal growth were recorded on days 4 and 7 following inoculation. Photographs were taken of the fungal colonies. Area of the fungal growth was determined by analyzing the photos using ImageJ software (FIGS. 28A-28H).

All species grew as expected on the water control Strep-PDA plates. By day 7, fast growing species had covered the entire plate (=56.7 sq. cm): Botrytis, Macrophomina. Rhizoctonia, Sclcrolium. Moderately fast-growing species were Colletotrichum (12.6 sq. cm), Fusarium (21.0 sq. cm), and Phytophthora (9.7 sq. cm). Verlicillium is a slow growing fungus and by day 7 reached 3.1 sq. cm (Table 2).

When treated with the biocontrol agent, Colletotrichum, Phytophthora, Rhizoctonia, Sclerotium, and Verticilium were all completely inhibited and showed no growth as of day 7 (=0.2 sq. cm, the area of the original agar plug). Compared to the water control, treatment with the biocontrol agent resulted in very limited growth of Botrytis (3.4 sq. cm), Fusarium (3.4 sq. cm), and Macrophomina (3.9 sq. cm).

### Example 9. Testing of Efficacy of Biocontrol Agents Produced at Different Time Points During the Aeration and Resting Phases of Example 1

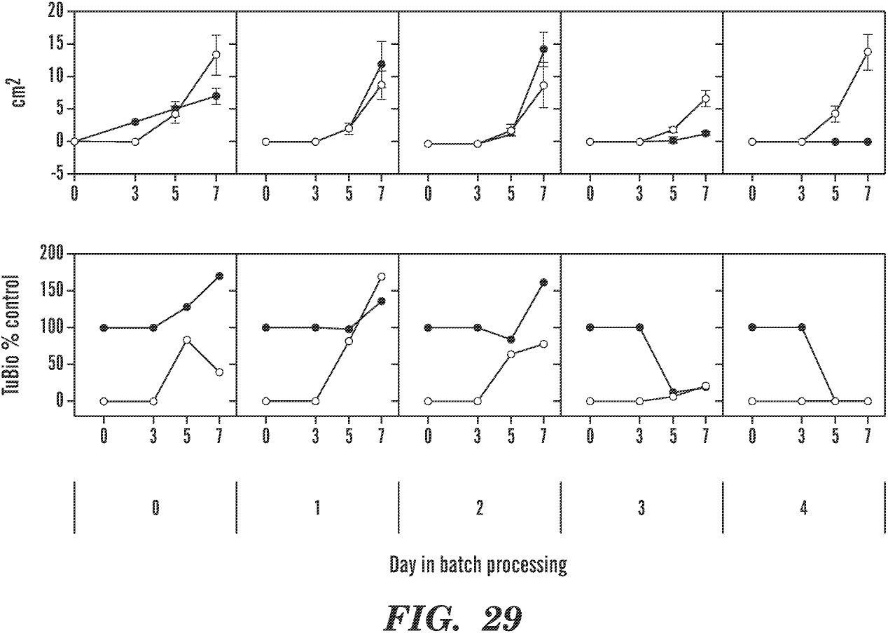

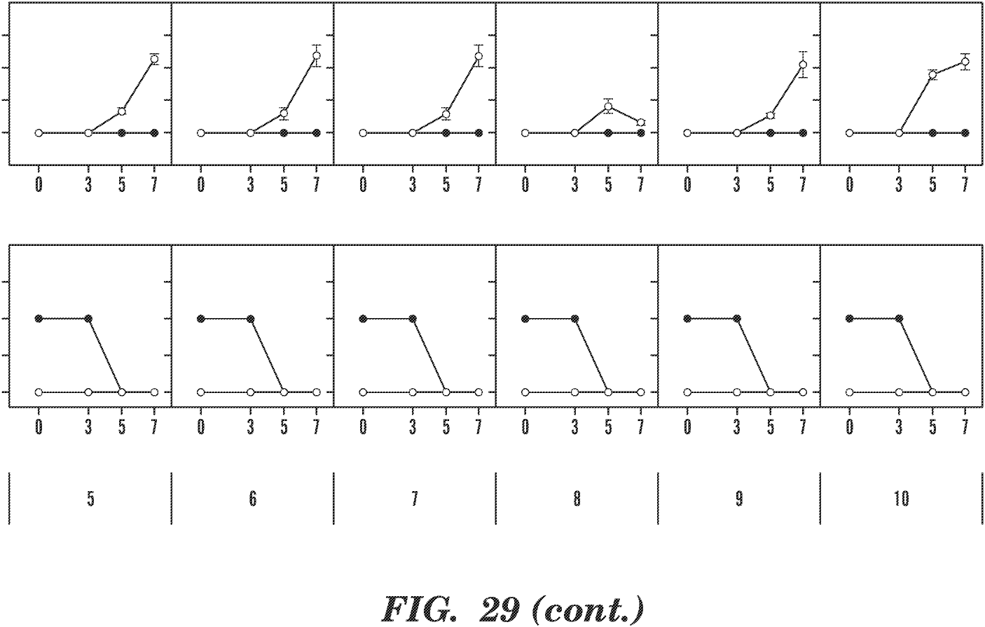

Efficacy for inhibition of Sclerotium by biocontrol agents produced at different time points during the production of a biocontrol agent according to the method of Example 1 was evaluated using a method similar to that described above in Examples 2, 3, and 8 (FIG. 29). It was observed that complete efficacy in inhibiting fungal growth was observed starting at Day 4 (day 1 of the resting phase) (FIG. 29).

### Other Embodiments

From the foregoing description, it will be apparent that variations and modifications may be made to the invention described herein to adapt it to various usages and conditions. Such embodiments are also within the scope of the following claims.

The recitation of a listing of elements in any definition of a variable herein includes definitions of that variable as any single element or combination (or subcombination) of listed elements The recitation of an embodiment herein includes that embodiment as any single embodiment or in combination with any other embodiments or portions thereof.

This application may be related in subject matter to the inventions described in U.S. Provisional Application No. 62/992364, the disclosure of which is incorporated herein by reference in its entirety for all purposes. All patents and publications mentioned in this specification are herein incorporated by reference to the same extent as if each independent patent and publication was specifically and individually indicated to be incorporated by reference.

1. A method for preparing a biocontrol agent, the method comprising:
a) aerobically incubating a mixture comprising a soil microbiome and a solution for at least about 1-3 days;
b) anaerobically incubating the mixture for at least about 1-3 days; and
c) removing solids from the mixture and retaining a conditioned media comprising soil microbiome metabolites, thereby preparing a biocontrol agent.

2. The method of claim 1, wherein the soil microbiome comprises a prokaryotic species relative abundance, as measured by 16S rRNA gene sequencing, of Proteobacteria, Firmicutes, and Actinobacteria of at least 30%.

3. The method of claim 1, wherein the soil microbiome comprises bacteria selected from the group consisting of Acidobacteria, Actinobacteria, Bacteroidetes, Chlamydiae, Chloroflexi, Cyanobacteria, Deinococcus-Thermus, Firmicutes, Gemmatimonadetes, Hydrogenedentes, Nitrospirae, Parcubacteria, Planctomycetes, Proteobacteria, Saccharibacteria, Spirochaetes, Tenericutes, Thaumarchaeota, and Verrucomicrobia.

4. A method for preparing a biocontrol agent, the method comprising:
a) aerobically incubating a mixture comprising a soil microbiome in solution, the soil microbiome comprising two or more bacteria selected from the group consisting of Acidobacteria, Actinobacteria, Bacteroidetes, Chlamydiae, Chloroflexi, Cyanobacteria, Deinococcus-Thermus, Firmicutes, Gemmatimonadetes, Hydrogenedentes, Nitrospirae, Parcubacteria, Planctomycetes, Proteobacteria, Saccharibacteria, Spirochaetes, Tenericutes, Thaumarchaeota, and Verrucomicrobia;
b) anaerobically incubating the soil microbiome in solution for at least about 1-3 days; and
c) removing microbial cells from the mixture and retaining a conditioned media comprising soil microbiome metabolites, thereby preparing a biocontrol agent.

5. (canceled)

6. The method of claim 1, wherein the soil microbiome is present in a solid matrix and the ratio of solid matrix to solution is at least about 1:10.

7-10. (canceled)

11. The method of claim 6, wherein the solid matrix is incubated in a bioreactor comprising a vessel having a perforated surface, the vessel comprising the solid matrix.

12-16. (canceled)

17. The method of claim 1 , wherein the sugar is added to the solution at the start of incubation, 1-3 days after the start of incubation, or periodically during the course of incubation.

18-19. (canceled)

20. The method of claim 1, wherein a gas comprising oxygen is introduced to the solution during the aerobic incubation.

21-27. (canceled)

28. The method of claim 1, wherein the pH of the mixture is neutral at the start of aerobic and/or anaerobic incubation.

29-31. (canceled)

32. The method of claim 1, wherein at least about 50% of bacteria present after the aerobic incubation and/or the anaerobic incubation, as measured by 16S rRNA gene sequencing, are Firmicutes and/or Gammaproteobacteria.

33. The method of claim 1, wherein the cell bath mixture comprises a prokaryotic species relative abundance, as measured by 16S rRNA gene sequencing, of Bacilli, Clostridia, and/or Gammaproteobacteria of at least about 20% after the aerobic incubation and/or the anaerobic incubation.

34. The method of claim 1, wherein the top 5 prokaryotic taxa represented in the cell bath mixture by relative abundance, as measured by 16S rRNA gene sequencing, comprises Bacillus, Clostridium, and Leuconostoc during or at the termination of the resting phase.

35. The method of claim 1, wherein the biocontrol agent comprises lactate, acetate, and propionate.

36. (canceled)

37. A biocontrol agent prepared by the method of claim 1.

38. A liquid biocontrol agent comprising metabolites of a soil microbiome, wherein the soil microbiome comprises two or more bacteria selected from the group consisting of Acidobacteria, Actinobacteria, Bacteroidetes, Chlamydiae, Chloroflexi, Cyanobacteria, Deinococcus-Thermus, Firmicutes, Gemmatimonadetes, Hydrogenedentes, Nitrospirae, Parcubacteria, Planctomycetes, Proteobacteria, Saccharibacteria, Spirochaetes, Tenericutes, Thaumarchaeota, and Verrucomicrobia, wherein the liquid biocontrol agent has anti-fungal activity.

39. (canceled)

40. A method of controlling a fungal pathogen, the method comprising contacting the fungal pathogen with a biocontrol agent of claim 37, thereby controlling the fungal pathogen.

41. A method of controlling a fungal pathogen, the method comprising contacting a soil or plant comprising the fungal pathogen with a biocontrol agent comprising metabolites of a soil microbiome, wherein the soil microbiome comprises two or more bacteria selected from the group consisting of Acidobacteria, Actinobacteria, Bacteroidetes, Chlamydiae, Chloroflexi, Cyanobacteria, Deinococcus-Thermus, Firmicutes, Gemmatimonadetes, Hydrogenedentes, Nitrospirae, Parcubacteria, Planctomycetes, Proteobacteria, Saccharibacteria, Spirochaetes, Tenericutes, Thaumarchaeota, and Verrucomicrobia.

42. (canceled)

43. The method of claim 41 ,wherein the plant belongs to the Allium genus.

44. (canceled)

45. The method of claim 41 , wherein the plant is selected from the group consisting of peas, lettuce, broccoli, beans, grape, strawberry, and raspberry.

46. The method of claim 40, wherein the fungal pathogen belongs to a genus selected from the group consisting of Botrytis, Colletotrichum, Fusarium, Macrophomina, Phytophthora, Pythium, Rhizoctonia, Sclerotinia, Sclerotiniaceae, Sclerotium, and Verticillium.

47. The method of claim 40, wherein the fungal pathogen is selected from the group consisting of Botrytis cinerea, Colletotrichum acutatum, Fusarium oxysporum sp. fragariae, Macrophomina phaseolina, Phytophthora cactorum, Pythium uncinulatum, Rhizoctonia solani, Sclerotinia sclerotiorum, Sclerotium cepivorum, Sclerotinia minor, and Verticillium dahliae.

48-54. (canceled)

55. A kit for use in the method of claim 1.

56. (canceled)
